(19) United States
(12) Patent Application Publication
(10) Pub. No.: US 2023/0057519 A1
(43) Pub. Date: Feb. 23, 2023

(54) Treatment of Ocular Diseases with Fully-Human Post-Translationally Modified Anti-VEGF Fab

(71) Applicant: REGENXBIO Inc., Rockville, MD (US)

(72) Inventors: Curran Matthew Simpson, Frederick, MD (US); Stephen Yoo, Newton, MA (US); Karen Fran Kozarsky, Bala Cynwyd, PA (US); Rickey Robert Reinhardt, Silver Spring, MD (US); Laura A. Coruzzi, New York, NY (US)

(73) Assignee: REGENXBIO Inc., Rockville, MD (US)

(21) Appl. No.: 17/704,170

(22) Filed: Mar. 25, 2022

Related U.S. Application Data
(63) Continuation of application No. 16/361,884, filed on Mar. 22, 2019, now abandoned.
Application No. 16/361,884 is a continuation of application No. 16/093,363, filed on Oct. 12, 2018, now abandoned.
(60) Provisional application No. 62/323,285, filed on Apr. 15, 2016.
(60) Provisional application No. 62/442,802, filed on Jan. 5, 2017.
(60) Provisional application No. 62/450,438, filed on Jan. 25, 2017.
(60) Provisional application No. 62/460,428, filed on Feb. 17, 2017.

(51) Int. Cl. C07K 16/22; A61K 48/00; C12N 15/86; A61P 27/02; A61K 9/00

(52) U.S. Cl. CPC C07K 16/22; A61K 48/0075; C12N 15/86; A61K 48/005; A61P 27/02; A61K 9/0048; C07K 2317/24; C07K 2317/41; C07K 2317/55; C12N 2750/14143; C12N 2830/42; C12N 2840/203; C12N 2830/50; A61K 2039/505

(57) ABSTRACT

Compositions and methods are described for the delivery of a fully human post-translationally modified (HuPTM) monoclonal antibody (“mAb”) or the antigen-binding fragment of a mAb against human vascular endothelial growth factor (“hVEGF”)—such as, e.g., a fully human-glycosylated (HuGly) anti-hVEGF antigen-binding fragment—to the retina/vitreal humour in the eye(s) of human subjects diagnosed with ocular diseases caused by increased neovascularization, for example, neovascular age-related macular degeneration (“nAMD”), also known as “wet” age-related macular degeneration (“WAMD”), age-related macular degeneration (“AMD”), and diabetic retinopathy.

This application claims the benefit of U.S. Provisional Application No. 62/323,285, filed Apr. 15, 2016, U.S. Provisional Application No. 62/442,802, filed Jan. 5, 2017, U.S. Provisional Application No. 62/450,438, filed Jan. 25, 2017, and U.S. Provisional Application No. 62/460,428, filed Feb. 17, 2017, each of which is hereby incorporated by reference in its entirety.

### REFERENCE TO SEQUENCE LISTING SUBMITTED ELECTRONICALLY

This application incorporates by reference a Sequence Listing submitted with this application as text file entitled “Sequence_Listing_12656-083-228.TXT” created on Apr. 13, 2017 and having a size of 21,394 bytes.

### 1. INTRODUCTION

Compositions and methods are described for the delivery of a fully human post-translationally modified (HuPTM) monoclonal antibody (“mAb”) or the antigen-binding fragment of a mAb against vascular endothelial growth factor (“VEGF”)—such as, e.g., a fully human-glycosylated (HuGly) anti-VEGF antigen-binding fragment—to the retina/vitreal humour in the eye(s) of human subjects diagnosed with ocular diseases caused by increased neovascularization, for example, neovascular age-related macular degeneration (“nAMD”), also known as “wet” age-related macular degeneration (“WAMD”), age-related macular degeneration (“AMD”), and diabetic retinopathy.

### 2. BACKGROUND OF THE INVENTION

Age-related macular degeneration (AMD) is a degenerative retinal eye disease that causes a progressive, irreversible, severe loss of central vision. The disease impairs the macula—the region of highest visual acuity (VA)—and is the leading cause of blindness in Americans 60 years or older (NIH 2008).

The “wet,” neovascular form of AMD (WAMD), also known as neovascular age-related macular degeneration (nAMD), accounts for 15-20% of AMD cases, and is characterized by abnormal neovascularization in and under the neuroretina in response to various stimuli. This abnormal vessel growth leads to formation of leaky vessels and often haemorrhage, as well as distortion and destruction of the normal retinal architecture. Visual function is severely impaired in nAMD, and eventually inflammation and scarring cause permanent loss of visual function in the affected retina. Ultimately, photoreceptor death and scar formation result in a severe loss of central vision and the inability to read, write, and recognize faces or drive. Many patients can no longer maintain gainful employment, carry out daily activities and consequently report a diminished quality of life (Mitchell, 2006).

Diabetic retinopathy is an ocular complication of diabetes, characterized by microaneurysms, hard exudates, hemorrhages, and venous abnormalities in the non-proliferative form and neovascularization, preretinal or vitreous hemorrhages, and fibrovascular proliferation in the proliferative form. Hyperglycemia induces microvascular retinal changes, leading to blurred vision, dark spots or flashing lights, and sudden loss of vision (Cai & McGinnis, 2016).

Preventative therapies have demonstrated little effect, and therapeutic strategies have focused primarily on treating the neovascular lesion. Available treatments for nAMD include laser photocoagulation, photodynamic therapy with verteporfin, and intravitreal (“IVT”) injections with agents aimed at binding to and neutralizing vascular endothelial growth factor (“VEGF”)—a cytokine implicated in stimulating angiogenesis and targeted for intervention. Such anti-VEGF agents used include, e.g., bevacizumab (a humanized monoclonal antibody (mAb) against VEGF produced in CHO cells), ranibizumab (the Fab portion of an affinity-improved variant of bevacizumab made in prokaryotic E. coli), aflibercept (a recombinant fusion protein consisting of VEGF-binding regions of the extracellular domains of the human VEGF-receptor fused to the Fc portion of human IgG1), or pegaptanib (a pegylated aptamer (a single-stranded nucleic acid molecule) that binds to VEGF). Each of these therapies has some effect on best-corrected visual acuity; however, their effects appear limited in restoring visual acuity and in duration.

Anti-VEGF IVT injections have been shown to be effective in reducing leakage and sometimes restoring visual loss. However, because these agents are effective for only a short period of time, repeated injections for long durations are often required, thereby creating considerable treatment burden for patients. While long term therapy with either monthly ranibizumab or monthly/every 8 week aflibercept may slow the progression of vision loss and improve vision, none of these treatments prevent neovascularization from recurring (Brown 2006; Rosenfeld, 2006; Schmidt-Erfurth, 2014). Each has to be re-administered to prevent the disease from worsening. The need for repeat treatments can incur additional risk to patients and is inconvenient for both patients and treating physicians.

### 3. SUMMARY OF THE INVENTION

Compositions and methods are described for the delivery of a fully human post-translationally modified (HuPTM) antigen-binding fragment of a monoclonal antibody (mAb) against VEGF (“HuPTMFabVEGFi”), for example, a fully human-glycosylated antigen-binding fragment of an anti-VEGF mAb (“HuGlyFabVEGFi”), to the retina/vitreal humour in the eye(s) of patients (human subjects) diagnosed with an ocular disease caused by increased neovascularization, for example, nAMD, also known as “wet” AMD. Such antigen-binding fragments include an Fab, F(ab′)₂, or scFv (single-chain variable fragment) of an anti-VEGF mAb (collectively referred to herein as “antigen-binding fragment”). In an alternative embodiment, full-length mAbs can be used. Delivery may be accomplished via gene therapy—e.g., by administering a viral vector or other DNA expression construct encoding an anti-VEGF antigen-binding fragment or mAb (or a hyperglycosylated derivative) to the subretinal and/or intraretinal space in the eye(s) of patients (human subjects) diagnosed with nAMD, to create a permanent depot in the eye that continuously supplies the human PTM, e.g., human-glycosylated, transgene product. The methods provided herein may also be used in patients (human subjects) diagnosed with AMD or diabetic retinopathy.

Described herein are anti-human vascular endothelial growth factor (hVEGF) antibodies, for example, anti-hVEGF antigen-binding fragments, produced by human retinal cells. Human VEGF (hVEGF) is a human protein encoded by the VEGFA gene. An exemplary amino acid sequence of hVEGF may be found at GenBank Accession No. AAA35789.1. An exemplary nucleic acid sequence of hVEGF may be found at GenBank Accession No. M32977.1.

In certain aspects, described herein are methods of treating a human subject diagnosed with neovascular age-related macular degeneration (nAMD), comprising delivering to the retina of said human subject a therapeutically effective amount of anti-hVEGF antigen-binding fragment produced by human retinal cells.

In certain aspects, described herein are methods of treating a human subject diagnosed with nAMD, comprising delivering to the retina of said human subject a therapeutically effective amount of anti-hVEGF antigen-binding fragment produced by human photoreceptor cells.

In certain aspects, described herein are methods of treating a human subject diagnosed with nAMD, comprising delivering to the eye of said human subject a therapeutically effective amount of anti-hVEGF antigen-binding fragment produced by human retinal cells.

In certain aspects, described herein are methods of treating a human subject diagnosed with nAMD, comprising delivering to the eye of said human subject a therapeutically effective amount of anti-hVEGF antigen-binding fragment produced by human photoreceptor cells.

In certain aspects of the methods described herein, the antigen-binding fragment comprises a heavy chain comprising the amino acid sequence of SEQ ID NO. 1 or SEQ ID NO. 3, and a light chain comprising the amino acid sequence of SEQ ID NO. 2, or SEQ ID NO. 4.

In certain aspects of the methods described herein, the antigen-binding fragment comprises light chain CDRs 1-3 of SEQ ID NOs: 14-16 and heavy chain CDRs 1-3 of SEQ ID NOs:17-19 or SEQ ID NOs: 20, 18, and 21.

In certain aspects, described herein are methods of treating a human subject diagnosed with neovascular age-related macular degeneration nAMD, comprising delivering to the eye of said human subject a therapeutically effective amount of anti-hVEGF antibody produced by human retinal cells.

In certain aspects, described herein are methods of treating a human subject diagnosed with nAMD, comprising delivering to the eye of said human subject a therapeutically effective amount of anti-hVEGF antibody produced by human photoreceptor cells.

In certain aspects, described herein are methods of treating a human subject diagnosed with nAMD, comprising delivering to the retina of said human subject a therapeutically effective amount of anti-hVEGF antibody produced by human retinal cells.

In certain aspects, described herein are methods of treating a human subject diagnosed with nAMD, comprising delivering to the retina of said human subject a therapeutically effective amount of anti-hVEGF antibody produced by human photoreceptor cells.

In certain aspects of the methods described herein, the antibody comprises a heavy chain comprising the amino acid sequence of SEQ ID NO. 1 or SEQ ID NO. 3, and a light chain comprising the amino acid sequence of SEQ ID NO. 2, or SEQ ID NO. 4.

In certain aspects of the methods described herein, the antibody comprises light chain CDRs 1-3 of SEQ ID NOs: 14-16 and heavy chain CDRs 1-3 of SEQ ID NOs:17-19 or SEQ ID NOs: 20, 18, and 21.

In certain aspects, described herein are methods of treating a human subject diagnosed with neovascular age-related macular degeneration (nAMD), comprising: delivering to the eye of said human subject, a therapeutically effective amount of an antigen-binding fragment of a mAb against hVEGF, said antigen-binding fragment containing a α2,6-sialylated glycan.

In certain aspects, described herein are methods of treating a human subject diagnosed with nAMD, comprising: delivering to the eye of said human subject, a therapeutically effective amount of a glycosylated antigen-binding fragment of a mAb against hVEGF, wherein said antigen-binding fragment does not contain NeuGc.

In certain aspects, described herein are methods of treating a human subject diagnosed with nAMD or age-related macular degeneration (AMD) or diabetic retinopathy, wherein the method comprises: administering to the subretinal space in the eye of said human subject an expression vector encoding an antigen-binding fragment of a mAb against hVEGF, wherein expression of said antigen-binding fragment is α2,6-sialylated upon expression from said expression vector in a human, immortalized retina-derived cell.

In certain aspects, described herein are methods of treating a human subject diagnosed with nAMD or AMD or diabetic retinopathy, wherein the method comprises: administering to the subretinal space in the eye of said human subject an expression vector encoding an antigen-binding fragment against hVEGF, wherein expression of said antigen-binding fragment is α2,6-sialylated upon expression from said expression vector in a human, immortalized retina-derived cell, wherein said antigen-binding fragment does not contain NeuGc.

In certain aspects, described herein are methods of treating a human subject diagnosed with nAMD, comprising: administering to the subretinal space in the eye of said human subject, a therapeutically effective amount of a recombinant nucleotide expression vector encoding an antigen-binding fragment of a mAb against hVEGF, so that a depot is formed that releases said antigen-binding fragment containing a α2,6-sialylated glycan.

In certain aspects, described herein are methods of treating a human subject diagnosed with nAMD, comprising: administering to the subretinal space in the eye of said human subject, a therapeutically effective amount of a recombinant nucleotide expression vector encoding an antigen-binding fragment of a mAb against hVEGF, so that a depot is formed that releases said antigen-binding fragment wherein said antigen-binding fragment is glycosylated but does not contain NeuGc.

In certain aspects of the methods described herein, the antigen-binding fragment comprises a heavy chain comprising the amino acid sequence of SEQ ID NO. 1 or SEQ ID NO. 3, and a light chain comprising the amino acid sequence of SEQ ID NO. 2, or SEQ ID NO. 4.

In certain aspects of the methods described herein, the antigen-binding fragment further contains a tyrosine-sulfation.

In certain aspects of the methods described herein, production of said antigen-binding fragment containing a α2,6-sialylated glycan is confirmed by transducing PER.C6 or RPE cell line with said recombinant nucleotide expression vector in cell culture.

In certain aspects of the methods described herein, production of said antigen-binding fragment containing a tyrosine-sulfation is confirmed by transducing PER.C6 or RPE cell line with said recombinant nucleotide expression vector in cell culture.

In certain aspects of the methods described herein, the vector has a hypoxia-inducible promoter.

In certain aspects of the methods described herein, the antigen-binding fragment comprises light chain CDRs 1-3 of SEQ ID NOs: 14-16 and heavy chain CDRs 1-3 of SEQ ID NOs:17-19 or SEQ ID NOs: 20, 18, and 21.

In certain aspects of the methods described herein, the antigen-binding fragment transgene encodes a leader peptide. A leader peptide may also be referred to as a signal peptide or leader sequence herein.

In certain aspects, described herein are methods of treating a human subject diagnosed with nAMD, comprising: administering to the subretinal space in the eye of said human subject, a therapeutically effective amount of a recombinant nucleotide expression vector encoding an antigen-binding fragment of a mAb against hVEGF, so that a depot is formed that releases said antigen-binding fragment containing a α2,6-sialylated glycan; wherein said recombinant vector, when used to transduce PER.C6 or RPE cells in culture results in production of said antigen-binding fragment containing a α2,6-sialylated glycan in said cell culture.

In certain aspects, described herein are methods of treating a human subject diagnosed with nAMD, comprising: administering to the subretinal space in the eye of said human subject, a therapeutically effective amount of a recombinant nucleotide expression vector encoding an antigen-binding fragment of a mAb against hVEGF, so that a depot is formed that releases said antigen-binding fragment wherein said antigen-binding fragment is glycosylated but does not contain NeuGc; wherein said recombinant vector, when used to transduce PER.C6 or RPE cells in culture results in production of said antigen-binding fragment that is glycosylated but does not contain NeuGc in said cell culture.

In certain aspects of the methods described herein, delivering to the eye comprises delivering to the retina, choroid, and/or vitreous humor of the eye.

In certain aspects of the methods described herein, the antigen-binding fragment comprises a heavy chain that comprises one, two, three, or four additional amino acids at the C-terminus.

In certain aspects of the methods described herein, the antigen-binding fragment comprises a heavy chain that does not comprise an additional amino acid at the C-terminus.

In certain aspects of the methods described herein produces a population of antigen-binding fragment molecules, wherein the antigen-binding fragment molecules comprise a heavy chain, and wherein 5%, 10%, or 20% of the population of antigen-binding fragment molecules comprises one, two, three, or four additional amino acids at the C-terminus of the heavy chain.

Subjects to whom such gene therapy is administered should be those responsive to anti-VEGF therapy. In particular embodiments, the methods encompass treating patients who have been diagnosed with nAMD and identified as responsive to treatment with an anti-VEGF antibody. In more specific embodiments, the patients are responsive to treatment with an anti-VEGF antigen-binding fragment. In certain embodiments, the patients have been shown to be responsive to treatment with an anti-VEGF antigen-binding fragment injected intravitreally prior to treatment with gene therapy. In specific embodiments, the patients have previously been treated with LUCENTIS® (ranibizumab), EYLEA® (aflibercept), and/or AVASTIN® (bevacizumab), and have been found to be responsive to one or more of said LUCENTIS® (ranibizumab), EYLEA® (aflibercept), and/or AVASTIN® (bevacizumab).

Subjects to whom such viral vector or other DNA expression construct is delivered should be responsive to the anti-hVEGF antigen-binding fragment encoded by the transgene in the viral vector or expression construct. To determine responsiveness, the anti-VEGF antigen-binding fragment transgene product (e.g., produced in cell culture, bioreactors, etc.) may be administered directly to the subject, such as by intravitreal injection.

The HuPTMFabVEGFi, e.g., HuGlyFabVEGFi, encoded by the transgene can include, but is not limited to an antigen-binding fragment of an antibody that binds to hVEGF, such as bevacizumab; an anti-hVEGF Fab moiety such as ranibizumab; or such bevacizumab or ranibizumab Fab moieties engineered to contain additional glycosylation sites on the Fab domain (e.g., see Courtois et al., 2016, mAbs 8: 99-112 which is incorporated by reference herein in its entirety for it description of derivatives of bevacizumab that are hyperglycosylated on the Fab domain of the full length antibody).

The recombinant vector used for delivering the transgene should have a tropism for human retinal cells or photoreceptor cells. Such vectors can include non-replicating recombinant adeno-associated virus vectors (“rAAV”), particularly those bearing an AAV8 capsid are preferred. However, other viral vectors may be used, including but not limited to lentiviral vectors, vaccinia viral vectors, or non-viral expression vectors referred to as “naked DNA” constructs. Preferably, the HuPTMFabVEGFi, e.g., HuGlyFabVEGFi, transgene should be controlled by appropriate expression control elements, for example, the CB7 promoter (a chicken β-actin promoter and CMV enhancer), the RPE65 promoter, or opsin promoter to name a few, and can include other expression control elements that enhance expression of the transgene driven by the vector (e.g., introns such as the chicken β-actin intron, minute virus of mice (MVM) intron, human factor IX intron (e.g., FIX truncated intron 1), β-globin splice donor/immunoglobulin heavy chain spice acceptor intron, adenovirus splice donor/immunoglobulin splice acceptor intron, SV40 late splice donor/splice acceptor (19S/16S) intron, and hybrid adenovirus splice donor/IgG splice acceptor intron and polyA signals such as the rabbit β-globin polyA signal, human growth hormone (hGH) polyA signal, SV40 late polyA signal, synthetic polyA (SPA) signal, and bovine growth hormone (bGH) polyA signal). See, e.g., Powell and Rivera-Soto, 2015, Discov. Med., 19(102):49-57.

Gene therapy constructs are designed such that both the heavy and light chains are expressed. More specifically, the heavy and light chains should be expressed at about equal amounts, in other words, the heavy and light chains are expressed at approximately a 1:1 ratio of heavy chains to light chains. The coding sequences for the heavy and light chains can be engineered in a single construct in which the heavy and light chains are separated by a cleavable linker or IRES so that separate heavy and light chain polypeptides are expressed. See, e.g., Section 5.2.4 for specific leader sequences and Section 5.2.5 for specific IRES, 2A, and other linker sequences that can be used with the methods and compositions provided herein.

Pharmaceutical compositions suitable for subretinal and/or intraretinal administration comprise a suspension of the recombinant (e.g., rHuGlyFabVEGFi) vector in a formulation buffer comprising a physiologically compatible aqueous buffer, a surfactant and optional excipients.

Therapeutically effective doses of the recombinant vector should be administered subretinally and/or intraretinally in an injection volume ranging from ≥0.1 mL to ≤0.5 mL, preferably in 0.1 to 0.30 mL (100-300 μ), and most preferably, in a volume of 0.25 mL (250 μl). Subretinal administration is a surgical procedure performed by trained retinal surgeons that involves a partial vitrectomy with the subject under local anesthesia, and injection of the gene therapy into the retina. (see, e.g., Campochiaro et al., 2016, Hum Gen Ther September 26 epub:doi:10.1089/hum.2016.117, which is incorporated by reference herein in its entirety). Subretinal and/or intraretinal administration should result in delivery of the soluble transgene product to the retina, the vitreous humor, and/or the aqueous humor. The expression of the transgene product (e.g., the encoded anti-VEGF antibody) by retinal cells, e.g., rod, cone, retinal pigment epithelial, horizontal, bipolar, amacrine, ganglion, and/or Müller cells, results in delivery and maintenance of the transgene product in the retina, the vitreous humor, and/or the aqueous humor. Doses that maintain a concentration of the transgene product at a Cₘᵢₙ of at least 0.330 μg/mL in the Vitreous humour, or 0.110 μg/mL in the Aqueous humour (the anterior chamber of the eye) for three months are desired; thereafter, Vitreous Cₘᵢₙ concentrations of the transgene product ranging from 1.70 to 6.60 μg/mL, and/or Aqueous Cₘᵢₙ concentrations ranging from 0.567 to 2.20 μg/mL should be maintained. However, because the transgene product is continuously produced, maintenance of lower concentrations can be effective. The concentration of the transgene product can be measured in patient samples of the vitreous humour and/or anterior chamber of the treated eye. Alternatively, vitreous humour concentrations can be estimated and/or monitored by measuring the patient's serum concentrations of the transgene product—the ratio of systemic to vitreal exposure to the transgene product is about 1:90,000. (E.g., see, vitreous humor and serum concentrations of ranibizumab reported in Xu L, et al., 2013, Invest. Opthal. Vis. Sci. 54: 1616-1624, at p. 1621 and Table 5 at p. 1623, which is incorporated by reference herein in its entirety).

The invention has several advantages over standard of care treatments that involve repeated ocular injections of high dose boluses of the VEGF inhibitor that dissipate over time resulting in peak and trough levels. Sustained expression of the transgene product antibody, as opposed to injecting an antibody repeatedly, allows for a more consistent levels of antibody to be present at the site of action, and is less risky and more convenient for patients, since fewer injections need to be made, resulting in fewer doctor visits. Furthermore, antibodies expressed from transgenes are post-translationally modified in a different manner than those that are directly injected because of the different microenvironment present during and after translation. Without being bound by any particular theory, this results in antibodies that have different diffusion, bioactivity, distribution, affinity, pharmacokinetic, and immunogenicity characteristics, such that the antibodies delivered to the site of action are “biobetters” in comparison with directly injected antibodies.

In addition, antibodies expressed from transgenes in vivo are not likely to contain degradation products associated with antibodies produced by recombinant technologies, such as protein aggregation and protein oxidation. Aggregation is an issue associated with protein production and storage due to high protein concentration, surface interaction with manufacturing equipment and containers, and purification with certain buffer systems. These conditions, which promote aggregation, do not exist in transgene expression in gene therapy. Oxidation, such as methionine, tryptophan, and histidine oxidation, is also associated with protein production and storage, and is caused by stressed cell culture conditions, metal and air contact, and impurities in buffers and excipients. The proteins expressed from transgenes in vivo may also oxidize in a stressed condition. However, humans, and many other organisms, are equipped with an antioxidation defense system, which not only reduces the oxidation stress, but sometimes also repairs and/or reverses the oxidation. Thus, proteins produced in vivo are not likely to be in an oxidized form. Both aggregation and oxidation could affect the potency, pharmacokinetics (clearance), and immunogenicity.

Without being bound by theory, the methods and compositions provided herein are based, in part, on the following principles:
(i) Human retinal cells are secretory cells that possess the cellular machinery for post-translational processing of secreted proteins—including glycosylation and tyrosine-O-sulfation, a robust process in retinal cells. (See, e.g., Wang et al., 2013, Analytical Biochem. 427: 20-28 and Adamis et al., 1993, BBRC 193: 631-638 reporting the production of glycoproteins by retinal cells; and Kanan et al., 2009, Exp. Eye Res. 89: 559-567 and Kanan & Al-Ubaidi, 2015, Exp. Eye Res. 133: 126-131 reporting the production of tyrosine-sulfated glycoproteins secreted by retinal cells, each of which is incorporated by reference in its entirety for post-translational modifications made by human retinal cells).
(ii) Contrary to the state of the art understanding, anti-VEGF antigen-binding fragments, such as ranibizumab (and the Fab domain of full length anti-VEGF mAbs such as Bevacizumab) do indeed possess N-linked glycosylation sites. For example, see FIG. 1 which identifies non-consensus asparaginal (“N”) glycosylation sites in the C_{H} domain (TVSWN¹⁶⁵SGAL) and in the C_{L} domain (QSGN¹⁵⁸SQE), as well as glutamine (“Q”) residues that are glycosylation sites in the V_{H} domain (Q¹¹⁵GT) and V_{L} domain (TFQ¹⁰⁰GT) of ranibizumab (and corresponding sites in the Fab of bevacizumab). (See, e.g., Valliere-Douglass et al., 2009, J. Biol. Chem. 284: 32493-32506, and Valliere-Douglass et al., 2010, J. Biol. Chem. 285: 16012-16022, each of which is incorporated by reference in its entirety for the identification of N-linked glycosylation sites in antibodies).
(iii) While such non-canonical sites usually result in low level glycosylation (e.g., about 1-5%) of the antibody population, the functional benefits may be significant in immunoprivileged organs, such as the eye (See, e.g., van de Bovenkamp et al., 2016, J. Immunol. 196:1435-1441). For example, Fab glycosylation may affect the stability, half-life, and binding characteristics of an antibody. To determine the effects of Fab glycosylation on the affinity of the antibody for its target, any technique known to one of skill in the art may be used, for example, enzyme linked immunosorbent assay (ELISA), or surface plasmon resonance (SPR). To determine the effects of Fab glycosylation on the half-life of the antibody, any technique known to one of skill in the art may be used, for example, by measurement of the levels of radioactivity in the blood or organs (e.g., the eye) in a subject to whom a radiolabelled antibody has been administered. To determine the effects of Fab glycosylation on the stability, for example, levels of aggregation or protein unfolding, of the antibody, any technique known to one of skill in the art may be used, for example, differential scanning calorimetry (DSC), high performance liquid chromatography (HPLC), e.g., size exclusion high performance liquid chromatography (SEC-HPLC), capillary electrophoresis, mass spectrometry, or turbidity measurement. Provided herein, the HuPTMFabVEGFi, e.g., HuGlyFabVEGFi, transgene results in production of a Fab which is 0.5%, 1%, 2%, 3%, 4%, 5%, 6%, 7%, 8%, 9%, or 10% or more glycosylated at non-canonical sites. In certain embodiments, 0.5%, 1%, 2%, 3%, 4%, 5%, 6%, 7%, 8%, 9%, or 10% or more Fabs from a population of Fabs are glycosylated at non-canonical sites. In certain embodiments, 0.5%, 1%, 2%, 3%, 4%, 5%, 6%, 7%, 8%, 9%, or 10% or more non-canonical sites are glycosylated. In certain embodiments, the glycosylation of the Fab at these non-canonical sites is 25%, 50%, 100%, 200%, 300%, 400%, 500%, or more greater than the amount of glycosylation of these non-canonical sites in a Fab produced in HEK293 cells.
(iv) In addition to the glycosylation sites, anti-VEGF Fabs such as ranibizumab (and the Fab of bevacizumab) contain tyrosine (“Y”) sulfation sites in or near the CDRs; see FIG. 1 which identifies tyrosine-O-sulfation sites in the V_{H} (EDTAVY⁹⁴Y⁹⁵) and V_{L} (EDFATY⁸⁶) domains of ranibizumab (and corresponding sites in the Fab of bevacizumab). (See, e.g., Yang et al., 2015, Molecules 20:2138-2164, esp. at p. 2154 which is incorporated by reference in its entirety for the analysis of amino acids surrounding tyrosine residues subjected to protein tyrosine sulfation. The “rules” can be summarized as follows: Y residues with E or D within +5 to −5 position of Y, and where position −1 of Y is a neutral or acidic charged amino acid—but not a basic amino acid, e.g., R, K, or H that abolishes sulfation). Human IgG antibodies can manifest a number of other post-translational modifications, such as N-terminal modifications, C-terminal modifications, degradation or oxidation of amino acid residues, cysteine related variants, and glycation (See, e.g., Liu et al., 2014, mAbs 6(5):1145-1154).
(v) Glycosylation of anti-VEGF Fabs, such as ranibizumab or the Fab fragment of bevacizumab by human retinal cells will result in the addition of glycans that can improve stability, half-life and reduce unwanted aggregation and/or immunogenicity of the transgene product. (See, e.g., Bovenkamp et al., 2016, J. Immunol. 196: 1435-1441 for a review of the emerging importance of Fab glycosylation). Significantly, glycans that can be added to HuPTMFabVEGFi, e.g., HuGlyFabVEGFi, provided herein, are highly processed complex-type biantennary N-glycans that contain 2,6-sialic acid (e.g., see FIG. 2 depicting the glycans that may be incorporated into HuPTMFabVEGFi, e.g., HuGlyFabVEGFi) and bisecting GlcNAc, but not NGNA. Such glycans are not present in ranibizumab (which is made in E. coli and is not glycosylated at all) or in bevacizumab (which is made in CHO cells that do not have the 2,6-sialyltransferase required to make this post-translational modification, nor do CHO cells product bisecting GlcNAc, although they do produce NGNA, which is immunogenic). See, e.g., Dumont et al., 2015, Crit. Rev. Biotechnol. (Early Online, published online September 18, 2015, pp. 1-13 at p. 5). The human glycosylation pattern of the HuPTMFabVEGFi, e.g., HuGlyFabVEGFi, provided herein, should reduce immunogenicity of the transgene product and improve efficacy.
(vi)Tyrosine-sulfation of anti-VEGF Fabs, such as ranibizumab or the Fab fragment of bevacizumab—a robust post-translational process in human retinal cells—could result in transgene products with increased avidity for VEGF. Indeed, tyrosine-sulfation of the Fab of therapeutic antibodies against other targets has been shown to dramatically increase avidity for antigen and activity. (See, e.g., Loos et al., 2015, PNAS 112: 12675-12680, and Choe et al., 2003, Cell 114: 161-170). Such post-translational modifications are not present on ranibizumab (which is made in E. coli a host that does not possess the enzymes required for tyrosine-sulfation), and at best is under-represented in bevacizumab—a CHO cell product. Unlike human retinal cells, CHO cells are not secretory cells and have a limited capacity for post-translational tyrosine-sulfation. (See, e.g., Mikkelsen & Ezban, 1991, Biochemistry 30: 1533-1537, esp. discussion at p. 1537).

For the foregoing reasons, the production of HuPTMFabVEGFi, e.g., HuGlyFabVEGFi, should result in a “biobetter” molecule for the treatment of nAMD accomplished via gene therapy—e.g., by administering a viral vector or other DNA expression construct encoding HuPTMFabVEGFi, e.g., HuGlyFabVEGFi, to the subretinal space in the eye(s) of patients (human subjects) diagnosed with nAMD, to create a permanent depot in the eye that continuously supplies the fully-human post-translationally modified, e.g., human-glycosylated, sulfated transgene product produced by transduced retinal cells. The cDNA construct for the FabVEGFi should include a signal peptide that ensures proper co- and post-translational processing (glycosylation and protein sulfation) by the transduced retinal cells. Such signal sequences used by retinal cells may include but are not limited to:

MNFLLSWVHW SLALLLYLHH AKWSQA
(VEGF-A signal peptide)
MERAAPSRRV PLPLLLLGGL ALLAAGVDA
(Fibulin-1 signal peptide)
MAPLRPLLIL ALLAWVALA
(Vitronectin signal peptide)
MRLLAKIICLMLWAICVA
(Complement Factor H signal peptide)
MRLLAFLSLL ALVLQETGT
(Opticin signal peptide)
MKWVTFISLLFLFSSAYS
(Albumin signal peptide)
MAFLWLLSCWALLGTTFG
(Chymotrypsinogen signal peptide)
MYRMQLLSCIALILALVTNS
(Interleukin-2 signal peptide)
MNLLLILTFVAAAVA
(Trypsinogen-2 signal peptide).

See, e.g., Stern et al., 2007, Trends Cell. Mol. Biol., 2:1-17 and Dalton & Barton, 2014, Protein Sci, 23: 517-525, each of which is incorporated by reference herein in its entirety for the signal peptides that can be used.

As an alternative, or an additional treatment to gene therapy, the HuPTMFabVEGFi product, e.g., HuGlyFabVEGFi glycoprotein, can be produced in human cell lines by recombinant DNA technology, and administered to patients diagnosed with nAMD by intravitreal injection. The HuPTMFabVEGFi product, e.g., glycoprotein, may also be administered to patients with AMD or diabetic retinopathy. Human cell lines that can be used for such recombinant glycoprotein production include but are not limited to human embryonic kidney 293 cells (HEK293), fibrosarcoma HT-1080, HKB-11, CAP, HuH-7, and retinal cell lines, PER.C6, or RPE to name a few (e.g., see Dumont et al., 2015, Crit. Rev. Biotechnol. (Early Online, published online September 18, 2015, pp. 1-13) “Human cell lines for biopharmaceutical manufacturing: history, status, and future perspectives” which is incorporated by reference in its entirety for a review of the human cell lines that could be used for the recombinant production of the HuPTMFabVEGFi product, e.g., HuGlyFabVEGFi glycoprotein). To ensure complete glycosylation, especially sialylation, and tyrosine-sulfation, the cell line used for production can be enhanced by engineering the host cells to co-express α-2,6-sialyltransferase (or both α-2,3- and α-2,6-sialyltransferases) and/or TPST-1 and TPST-2 enzymes responsible for tyrosine-O-sulfation in retinal cells.

Combinations of delivery of the HuPTMFabVEGFi, e.g., HuGlyFabVEGFi, to the eye/retina accompanied by delivery of other available treatments are encompassed by the methods provided herein. The additional treatments may be administered before, concurrently or subsequent to the gene therapy treatment. Available treatments for nAMD that could be combined with the gene therapy provided herein include but are not limited to laser photocoagulation, photodynamic therapy with verteporfin, and intravitreal (IVT) injections with anti-VEGF agents, including but not limited to pegaptanib, ranibizumab, aflibercept, or bevacizumab. Additional treatments with anti-VEGF agents, such as biologics, may be referred to as “rescue” therapy.

Unlike small molecule drugs, biologics usually comprise a mixture of many variants with different modifications or forms that have a different potency, pharmacokinetics, and safety profile. It is not essential that every molecule produced either in the gene therapy or protein therapy approach be fully glycosylated and sulfated. Rather, the population of glycoproteins produced should have sufficient glycosylation (from about 1% to about 10% of the population), including 2,6-sialylation, and sulfation to demonstrate efficacy. The goal of gene therapy treatment provided herein is to slow or arrest the progression of retinal degeneration, and to slow or prevent loss of vision with minimal intervention/invasive procedures. Efficacy may be monitored by measuring BCVA (Best-Corrected Visual Acuity), intraocular pressure, slit lamp biomicroscopy, indirect ophthalmoscopy, SD-OCT (SD-Optical Coherence Tomography), electroretinography (ERG). Signs of vision loss, infection, inflammation and other safety events, including retinal detachment may also be monitored. Retinal thickness may be monitored to determine efficacy of the treatments provided herein. Without being bound by any particular theory, thickness of the retina may be used as a clinical readout, wherein the greater reduction in retinal thickness or the longer period of time before thickening of the retina, the more efficacious the treatment. Retinal thickness may be determined, for example, by SD-OCT. SD-OCT is a three-dimensional imaging technology which uses low-coherence interferometry to determine the echo time delay and magnitude of backscattered light reflected off an object of interest. OCT can be used to scan the layers of a tissue sample (e.g., the retina) with 3 to 15 μm axial resolution, and SD-OCT improves axial resolution and scan speed over previous forms of the technology (Schuman, 2008, Trans. Am. Opthamol. Soc. 106:426-458). Retinal function may be determined, for example, by ERG. ERG is a non-invasive electrophysiologic test of retinal function, approved by the FDA for use in humans, which examines the light sensitive cells of the eye (the rods and cones), and their connecting ganglion cells, in particular, their response to a flash stimulation.

Unexpected benefits of the invention are illustrated in the examples, infra, which demonstrate that expression of the HuPTMFabVEGFi from a rAAV8.anti-hVEGF Fab vector injected into the subretinal space (i) reduced subretinal neovascularization in transgenic mice that are models of nAMD in human subjects; and (ii) and surprisingly prevented retinal detachment in a transgenic mouse model of ocular neovascular disease that develops severe proliferative retinopathy and retinal detachment caused by ocular production of VEGF.

### ILLUSTRATIVE EMBODIMENTS

1. A method of treating a human subject diagnosed with nAMD, comprising delivering to the retina of said human subject a therapeutically effective amount of anti-hVEGF antigen-binding fragment produced by human retinal cells.

2. A method of treating a human subject diagnosed with nAMD, comprising delivering to the retina of said human subject a therapeutically effective amount of anti-hVEGF antigen-binding fragment produced by human photoreceptor cells.

3. The method of paragraph 1 or 2, in which the antigen-binding fragment is a Fab.

4. The method of paragraph 1 or 2, in which the antigen-binding fragment is an F(ab′)₂.

5. The method of paragraph 1 or 2, in which the antigen-binding fragment is a single chain variable domain (scFv).

6. The method of paragraph 1 or 2, in which the antigen-binding fragment comprises a heavy chain comprising the amino acid sequence of SEQ ID NO. 1 or SEQ ID NO. 3, and a light chain comprising the amino acid sequence of SEQ ID NO. 2, or SEQ ID NO. 4.

7. The method of paragraph 1 or 2, wherein the antigen-binding fragment comprises light chain CDRs 1-3 of SEQ ID NOs: 14-16 and heavy chain CDRs 1-3 of SEQ ID NOs:17-19 or SEQ ID NOs: 20, 18, and 21.

8. A method of treating a human subject diagnosed with neovascular age-related macular degeneration (nAMD), comprising delivering to the eye of said human subject, a therapeutically effective amount of an antigen-binding fragment (a Fab, F(ab′)₂, or an scFv, collectively referred to herein as an “antigen-binding fragment”) of a mAb against hVEGF, said antigen-binding fragment containing a α2,6-sialylated glycan.

9. A method of treating a human subject diagnosed with nAMD, comprising delivering to the eye of said human subject, a therapeutically effective amount of a glycosylated antigen-binding fragment of a mAb against hVEGF, wherein said antigen-binding fragment does not contain NeuGc.

10. A method of treating a human subject diagnosed with nAMD or AMD or diabetic retinopathy, wherein the method comprises: administering to the subretinal space in the eye of said human subject a expression vector encoding an antigen-binding fragment against hVEGF, wherein expression of said antigen-binding fragment is α2,6-sialylated upon expression from said expression vector in a human, immortalized retina-derived cell.

11. A method of treating a human subject diagnosed with nAMD or AMD or diabetic retinopathy, wherein the method comprises: administering to the subretinal space in the eye of said human subject a expression vector encoding an antigen-binding fragment against hVEGF, wherein expression of said antigen-binding fragment is α2,6-sialylated upon expression from said expression vector in a human, immortalized retina-derived cell, wherein said antigen-binding fragment does not contain NeuGc.

12. A method of treating a human subject diagnosed with nAMD, comprising administering to the subretinal space in the eye of said human subject, a therapeutically effective amount of a recombinant nucleotide expression vector encoding an antigen-binding fragment of a mAb against hVEGF, so that a depot is formed that releases said antigen-binding fragment containing a α2,6-sialylated glycan.

13. A method of treating a human subject diagnosed with nAMD, comprising administering to the subretinal space in the eye of said human subject, a therapeutically effective amount of a recombinant nucleotide expression vector encoding an antigen-binding fragment of a mAb against hVEGF, so that a depot is formed that releases said antigen-binding fragment wherein said antigen-binding fragment is glycosylated but does not contain NeuGc.

14. The method of any one of paragraphs 8 to 13 in which the antigen-binding fragment comprises a heavy chain comprising the amino acid sequence of SEQ ID NO. 1 or SEQ ID NO. 3, and a light chain comprising the amino acid sequence of SEQ ID NO. 2, or SEQ ID NO. 4.

15. The method of any one of paragraphs 8 to 14, in which the antigen-binding fragment further contains a tyrosine-sulfation.

16. The method of any one of paragraphs 8 to 15 in which production of said antigen-binding fragment containing a α2,6-sialylated glycan is confirmed by transducing PER.C6 or RPE cell line with said recombinant nucleotide expression vector in cell culture.

17. The method of any one of paragraphs 8 to 15 in which production of said antigen-binding fragment containing a tyrosine-sulfation is confirmed by transducing PER.C6 or RPE cell line with said recombinant nucleotide expression vector in cell culture.

18. The method of any one of paragraphs 8 to 17, wherein the vector has a hypoxia-inducible promoter.

19. The method of any one of paragraphs 8 to 18, wherein the antigen-binding fragment comprises light chain CDRs 1-3 of SEQ ID NOs: 14-16 and heavy chain CDRs 1-3 of SEQ ID NOs:17-19 or SEQ ID NOs: 20, 18, and 21.

20. The method of any one of paragraphs 8 to 19, wherein the antigen-binding fragment transgene encodes a leader peptide.

21. A method of treating a human subject diagnosed with nAMD, comprising administering to the subretinal space in the eye of said human subject, a therapeutically effective amount of a recombinant nucleotide expression vector encoding an antigen-binding fragment of a mAb against hVEGF, so that a depot is formed that releases said antigen-binding fragment containing a α2,6-sialylated glycan; wherein said recombinant vector, when used to transduce PER.C6 or RPE cells in culture results in production of said antigen-binding fragment containing a α2,6-sialylated glycan in said cell culture.

22. A method of treating a human subject diagnosed with nAMD, comprising administering to the subretinal space in the eye of said human subject, a therapeutically effective amount of a recombinant nucleotide expression vector encoding an antigen-binding fragment of a mAb against hVEGF, so that a depot is formed that releases said antigen-binding fragment wherein said antigen-binding fragment is glycosylated but does not contain NeuGc; wherein said recombinant vector, when used to transduce PER.C6 or RPE cells in culture results in production of said antigen-binding fragment that is glycosylated but does not contain NeuGc in said cell culture.

23. The method of paragraph 1 or paragraph 2, wherein delivering to the eye comprises delivering to the retina, choroid, and/or vitreous humor of the eye.

24. The method of any one of paragraphs 1 to 23, wherein the antigen-binding fragment comprises a heavy chain that comprises one, two, three, or four additional amino acids at the C-terminus.

25. The method of any one of paragraphs 1 to 23, wherein the antigen-binding fragment comprises a heavy chain that does not comprise an additional amino acid at the C-terminus.

26. The method of any one of paragraphs 1 to 23, which produces a population of antigen-binding fragment molecules, wherein the antigen-binding fragment molecules comprise a heavy chain, and wherein 5%, 10%, or 20% of the population of antigen-binding fragment molecules comprises one, two, three, or four additional amino acids at the C-terminus of the heavy chain.

### 4. BRIEF DESCRIPTION OF THE DRAWINGS

FIG. 1. The amino acid sequence of Ranibizumab (top) showing 5 different residues in Bevacizumab Fab (below). The starts of the variable and constant heavy chains (V_{H} and C_{H}) and light chains (V_{L} and V_{C}) are indicated by arrows (→), and the CDRs are underscored. Non-consensus glycosylation sites (“Gsite”) tyrosine-O-sulfation sites (“Ysite”) are indicated.

FIG. 2. Glycans that can be attached to HuGlyFabVEGFi. (Adapted from Bondt et al., 2014, Mol & Cell Proteomics 13.1: 3029-3039).

FIG. 3. The amino acid sequence of hyperglycosylated variants of Ranibizumab (above) and Bevacizumab Fab (below). The starts of the variable and constant heavy chains (V_{H} and C_{H}) and light chains (V_{L} and V_{C}) are indicated by arrows (→), and the CDRs are underscored. Non-consensus glycosylation sites (“Gsite”) and tyrosine-O-sulfation sites (“Ysite”) are indicated. Four hyperglycoslated variants are indicated with an asterisk (*).

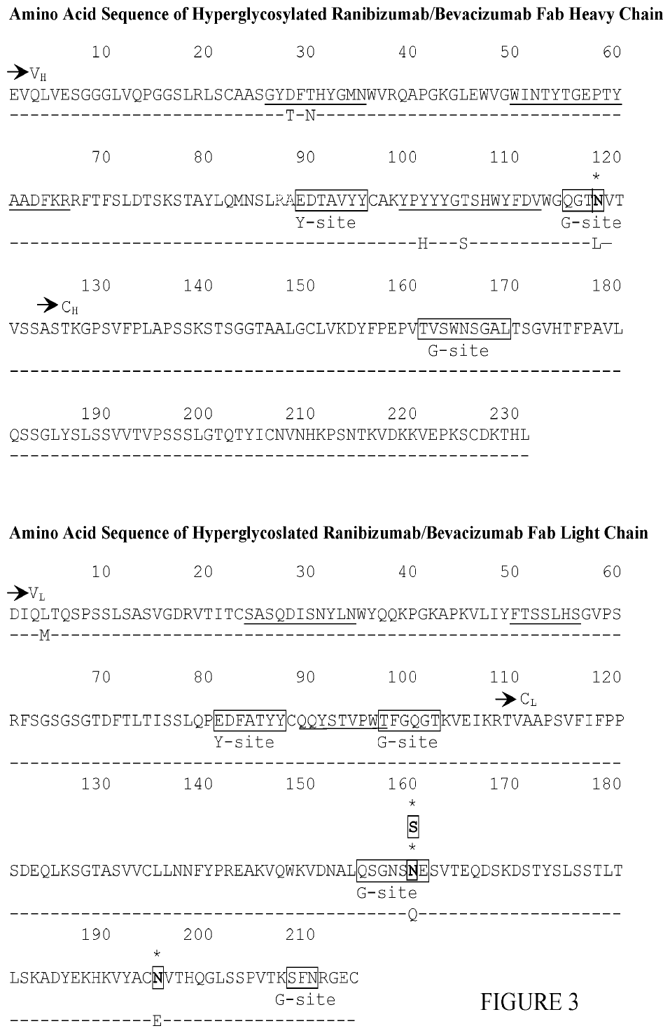

FIG. 4. Dose-dependent reduction in neovascular area in Rho/VEGF Mice administered subretinal injections of Vector 1. Rho/VEGF mice were injected subretinally with the indicated doses of Vector 1 or control (PBS or empty vector at 1×10¹⁰ GC/eye), and one week later the area of retinal neovascularization was quantitated. The numbers of mice/group are designated on each bar. * indicates a p value between 0.0019 and 0.0062; ** indicates of a p value <0.0001.

FIG. 5. Reduction in the incidence and severity of retinal detachment in Tet/Opsin/VEGF mice administered subretinal injections of Vector 1. Tet/opsin/VEGF mice were injected subretinally with the indicated doses of Vector 1 or control (PBS or empty vector at 1×10¹⁰ GC/eye). Ten days later, VEGF expression was induced with the addition of doxycycline to the drinking water, and after 4 days, eyes were assessed for the presence of full retinal detachment, partial detachment, or no detachment.

### 5. DETAILED DESCRIPTION OF THE INVENTION

Compositions and methods are described for the delivery of a fully human post-translationally modified (HuPTM) antigen-binding fragment of a monoclonal antibody (mAb) against VEGF (“HuPTMFabVEGFi”), for example, a fully human-glycosylated antigen-binding fragment of an anti-VEGF mAb (“HuGlyFabVEGFi”), to the retina/vitreal humour in the eye(s) of patients (human subjects) diagnosed with an ocular disease caused by increased neovascularization, for example, nAMD, also known as “wet” AMD. Such antigen-binding fragments include a Fab, F(ab′)₂, or scFv (single-chain variable fragment) of an anti-VEGF mAb (collectively referred to herein as “antigen-binding fragment”). In an alternative embodiment, full-length mAbs can be used. Delivery may be accomplished via gene therapy—e.g., by administering a viral vector or other DNA expression construct encoding an anti-VEGF antigen-binding fragment or mAb (or a hyperglycosylated derivative) to the subretinal and/or intraretinal space in the eye(s) of patients (human subjects) diagnosed with nAMD, to create a permanent depot in the eye that continuously supplies the human PTM, e.g., human-glycosylated, transgene product. The methods provided herein may also be used in patients (human subjects) diagnosed with AMD or diabetic retinopathy.

Subjects to whom such gene therapy is administered should be those responsive to anti-VEGF therapy. In particular embodiments, the methods encompass treating patients who have been diagnosed with nAMD and identified as responsive to treatment with an anti-VEGF antibody. In more specific embodiments, the patients are responsive to treatment with an anti-VEGF antigen-binding fragment. In certain embodiments, the patients have been shown to be responsive to treatment with an anti-VEGF antigen-binding fragment injected intravitreally prior to treatment with gene therapy. In specific embodiments, the patients have previously been treated with LUCENTIS® (ranibizumab), EYLEA® (aflibercept), and/or AVASTIN® (bevacizumab), and have been found to be responsive to one or more of said LUCENTIS® (ranibizumab), EYLEA® (aflibercept), and/or AVASTIN® (bevacizumab).

Subjects to whom such viral vector or other DNA expression construct is delivered should be responsive to the anti-VEGF antigen-binding fragment encoded by the transgene in the viral vector or expression construct. To determine responsiveness, the anti-hVEGF antigen-binding fragment transgene product (e.g., produced in cell culture, bioreactors, etc.) may be administered directly to the subject, such as by intravitreal injection.

The HuPTMFabVEGFi, e.g., HuGlyFabVEGFi, encoded by the transgene can include, but is not limited to an antigen-binding fragment of an antibody that binds to hVEGF, such as bevacizumab; an anti-hVEGF Fab moiety such as ranibizumab; or such bevacizumab or ranibizumab Fab moieties engineered to contain additional glycosylation sites on the Fab domain (e.g., see Courtois et al., 2016, mAbs 8: 99-112 which is incorporated by reference herein in its entirety for it description of derivatives of bevacizumab that are hyperglycosylated on the Fab domain of the full length antibody).

The recombinant vector used for delivering the transgene should have a tropism for human retinal cells or photoreceptor cells. Such vectors can include non-replicating recombinant adeno-associated virus vectors (“rAAV”), particularly those bearing an AAV8 capsid are preferred. However, other viral vectors may be used, including but not limited to lentiviral vectors, vaccinia viral vectors, or non-viral expression vectors referred to as “naked DNA” constructs. Preferably, the HuPTMFabVEGFi, e.g., HuGlyFabVEGFi, transgene should be controlled by appropriate expression control elements, for example, the CB7 promoter (a chicken β-actin promoter and CMV enhancer), the RPE65 promoter, or opsin promoter to name a few, and can include other expression control elements that enhance expression of the transgene driven by the vector (e.g., introns such as the chicken β-actin intron, minute virus of mice (MVM) intron, human factor IX intron (e.g., FIX truncated intron 1), β-globin splice donor/immunoglobulin heavy chain spice acceptor intron, adenovirus splice donor/immunoglobulin splice acceptor intron, SV40 late splice donor/splice acceptor (19S/16S) intron, and hybrid adenovirus splice donor/IgG splice acceptor intron and polyA signals such as the rabbit β-globin polyA signal, human growth hormone (hGH) polyA signal, SV40 late polyA signal, synthetic polyA (SPA) signal, and bovine growth hormone (bGH) polyA signal). See, e.g., Powell and Rivera-Soto, 2015, Discov. Med., 19(102):49-57.

Gene therapy constructs are designed such that both the heavy and light chains are expressed. More specifically, the heavy and light chains should be expressed at about equal amounts, in other words, the heavy and light chains are expressed at approximately a 1:1 ratio of heavy chains to light chains. The coding sequences for the heavy and light chains can be engineered in a single construct in which the heavy and light chains are separated by a cleavable linker or IRES so that separate heavy and light chain polypeptides are expressed. See, e.g., Section 5.2.4 for specific leader sequences and Section 5.2.5 for specific IRES, 2A, and other linker sequences that can be used with the methods and compositions provided herein.

Pharmaceutical compositions suitable for subretinal and/or intraretinal administration comprise a suspension of the recombinant (e.g., rHuGlyFabVEGFi) vector in a formulation buffer comprising a physiologically compatible aqueous buffer, a surfactant and optional excipients.

Therapeutically effective doses of the recombinant vector should be administered subretinally and/or intraretinally in an injection volume ranging from ≥0.1 mL to ≤0.5 mL, preferably in 0.1 to 0.30 mL (100-300 μl), and most preferably, in a volume of 0.25 mL (250 μl). Subretinal administration is a surgical procedure performed by trained retinal surgeons that involves a partial vitrectomy with the subject under local anesthesia, and injection of the gene therapy into the retina. (see, e.g., Campochiaro et al., 2016, Hum Gen Ther September 26 epub:doi:10.1089/hum.2016.117, which is incorporated by reference herein in its entirety). Subretinal and/or intraretinal administration should result in delivery of the soluble transgene product to the retina, the vitreous humor, and/or the aqueous humor. The expression of the transgene product (e.g., the encoded anti-VEGF antibody) by retinal cells, e.g., rod, cone, retinal pigment epithelial, horizontal, bipolar, amacrine, ganglion, and/or Müller cells, results in delivery and maintenance of the transgene product in the retina, the vitreous humor, and/or the aqueous humor. Doses that maintain a concentration of the transgene product at a Cₘᵢₙ of at least 0.330 μg/mL in the Vitreous humour, or 0.110 μg/mL in the Aqueous humour (the anterior chamber of the eye) for three months are desired; thereafter, Vitreous Cₘᵢₙ concentrations of the transgene product ranging from 1.70 to 6.60 μg/mL, and/or Aqueous Cₘᵢₙ concentrations ranging from 0.567 to 2.20 μg/mL should be maintained. However, because the transgene product is continuously produced, maintenance of lower concentrations can be effective. The concentration of the transgene product can be measured in patient samples of the vitreous humour and/or anterior chamber of the treated eye. Alternatively, vitreous humour concentrations can be estimated and/or monitored by measuring the patient's serum concentrations of the transgene product—the ratio of systemic to vitreal exposure to the transgene product is about 1:90,000. (E.g., see, vitreous humor and serum concentrations of ranibizumab reported in Xu L, et al., 2013, Invest. Opthal. Vis. Sci. 54: 1616-1624, at p. 1621 and Table 5 at p. 1623, which is incorporated by reference herein in its entirety).

The invention has several advantages over standard of care treatments that involve repeated ocular injections of high dose boluses of the VEGF inhibitor that dissipate over time resulting in peak and trough levels. Sustained expression of the transgene product antibody, as opposed to injecting an antibody repeatedly, allows for a more consistent levels of antibody to be present at the site of action, and is less risky and more convenient for patients, since fewer injections need to be made, resulting in fewer doctor visits. Furthermore, antibodies expressed from transgenes are post-translationally modified in a different manner than those that are directly injected because of the different microenvironment present during and after translation. Without being bound by any particular theory, this results in antibodies that have different diffusion, bioactivity, distribution, affinity, pharmacokinetic, and immunogenicity characteristics, such that the antibodies delivered to the site of action are “biobetters” in comparison with directly injected antibodies.

In addition, antibodies expressed from transgenes in vivo are not likely to contain degradation products associated with antibodies produced by recombinant technologies, such as protein aggregation and protein oxidation. Aggregation is an issue associated with protein production and storage due to high protein concentration, surface interaction with manufacturing equipment and containers, and purification with certain buffer systems. These conditions, which promote aggregation, do not exist in transgene expression in gene therapy. Oxidation, such as methionine, tryptophan, and histidine oxidation, is also associated with protein production and storage, and is caused by stressed cell culture conditions, metal and air contact, and impurities in buffers and excipients. The proteins expressed from transgenes in vivo may also oxidize in a stressed condition. However, humans, and many other organisms, are equipped with an antioxidation defense system, which not only reduces the oxidation stress, but sometimes also repairs and/or reverses the oxidation. Thus, proteins produced in vivo are not likely to be in an oxidized form. Both aggregation and oxidation could affect the potency, pharmacokinetics (clearance), and immunogenicity.

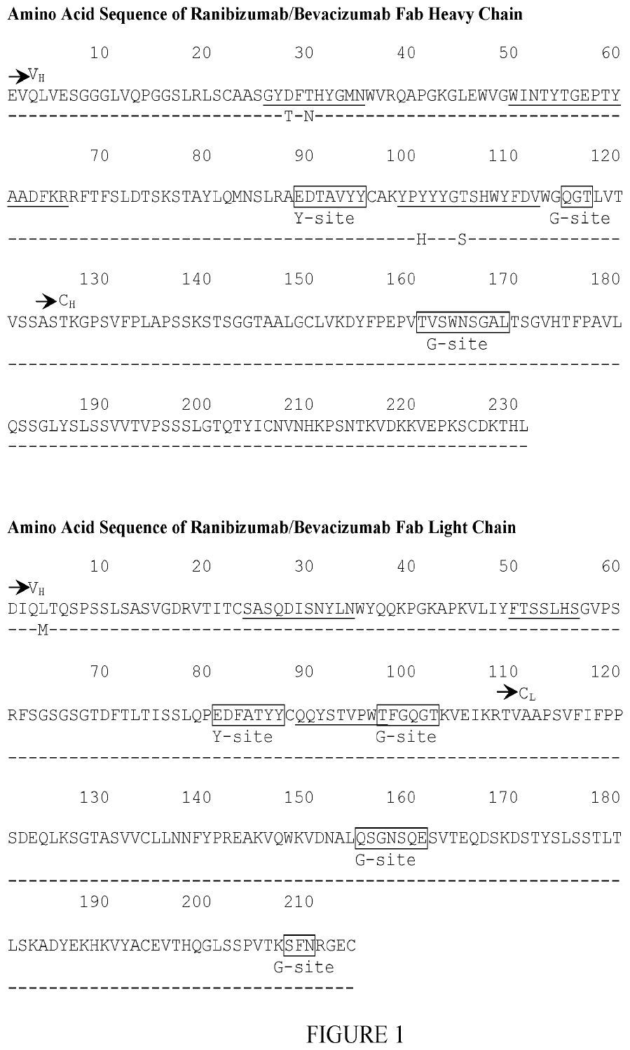

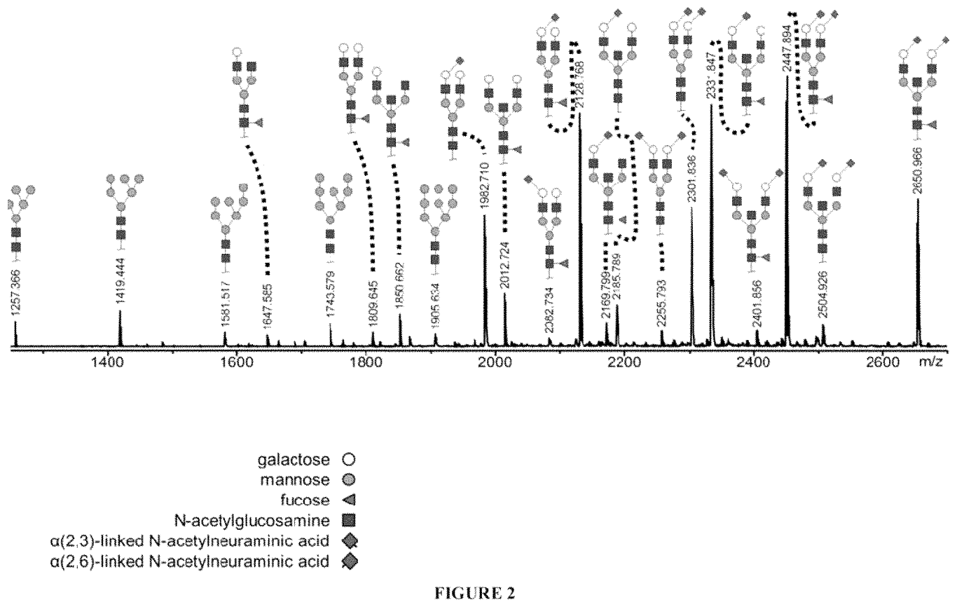

Without being bound by theory, the methods and compositions provided herein are based, in part, on the following principles:
(i) Human retinal cells are secretory cells that possess the cellular machinery for post-translational processing of secreted proteins — including glycosylation and tyrosine-O-sulfation, a robust process in retinal cells. (See, e.g., Wang et al., 2013, Analytical Biochem. 427: 20-28 and Adamis et al., 1993, BBRC 193: 631-638 reporting the production of glycoproteins by retinal cells; and Kanan et al., 2009, Exp. Eye Res. 89: 559-567 and Kanan & Al-Ubaidi, 2015, Exp. Eye Res. 133: 126-131 reporting the production of tyrosine-sulfated glycoproteins secreted by retinal cells, each of which is incorporated by reference in its entirety for post-translational modifications made by human retinal cells).
(ii) Contrary to the state of the art understanding, anti-VEGF antigen-binding fragments, such as ranibizumab (and the Fab domain of full length anti-VEGF mAbs such as Bevacizumab) do indeed possess N-linked glycosylation sites. For example, see FIG. 1 which identifies non-consensus asparaginal (“N”) glycosylation sites in the C_{H} domain (TVSWN¹⁶⁵SGAL) and in the C_{L} domain (QSGN¹⁵⁸SQE), as well as glutamine (“Q”) residues that are glycosylation sites in the V_{H} domain (Q¹¹⁵GT) and V_{L} domain (TFQ¹⁰⁰GT) of ranibizumab (and corresponding sites in the Fab of bevacizumab). (See, e.g., Valliere-Douglass et al., 2009, J. Biol. Chem. 284: 32493-32506, and Valliere-Douglass et al., 2010, J. Biol. Chem. 285: 16012-16022, each of which is incorporated by reference in its entirety for the identification of N-linked glycosylation sites in antibodies).
(iii) While such non-canonical sites usually result in low level glycosylation (e.g., about 1-5%) of the antibody population, the functional benefits may be significant in immunoprivileged organs, such as the eye (See, e.g., van de Bovenkamp et al., 2016, J. Immunol. 196:1435-1441). For example, Fab glycosylation may affect the stability, half-life, and binding characteristics of an antibody. To determine the effects of Fab glycosylation on the affinity of the antibody for its target, any technique known to one of skill in the art may be used, for example, enzyme linked immunosorbent assay (ELISA), or surface plasmon resonance (SPR). To determine the effects of Fab glycosylation on the half-life of the antibody, any technique known to one of skill in the art may be used, for example, by measurement of the levels of radioactivity in the blood or organs (e.g., the eye) in a subject to whom a radiolabelled antibody has been administered. To determine the effects of Fab glycosylation on the stability, for example, levels of aggregation or protein unfolding, of the antibody, any technique known to one of skill in the art may be used, for example, differential scanning calorimetry (DSC), high performance liquid chromatography (HPLC), e.g., size exclusion high performance liquid chromatography (SEC-HPLC), capillary electrophoresis, mass spectrometry, or turbidity measurement. Provided herein, the HuPTMFabVEGFi, e.g., HuGlyFabVEGFi, transgene results in production of a Fab which is 0.5%, 1%, 2%, 3%, 4%, 5%, 6%, 7%, 8%, 9%, or 10% or more glycosylated at non-canonical sites. In certain embodiments, 0.5%, 1%, 2%, 3%, 4%, 5%, 6%, 7%, 8%, 9%, or 10% or more Fabs from a population of Fabs are glycosylated at non-canonical sites. In certain embodiments, 0.5%, 1%, 2%, 3%, 4%, 5%, 6%, 7%, 8%, 9%, or 10% or more non-canonical sites are glycosylated. In certain embodiments, the glycosylation of the Fab at these non-canonical sites is 25%, 50%, 100%, 200%, 300%, 400%, 500%, or more greater than the amount of glycosylation of these non-canonical sites in a Fab produced in HEK293 cells.
(iv) In addition to the glycosylation sites, anti-VEGF Fabs such as ranibizumab (and the Fab of bevacizumab) contain tyrosine (“Y”) sulfation sites in or near the CDRs; see FIG. 1 which identifies tyrosine-O-sulfation sites in the V_{H} (EDTAVY⁹⁴Y⁹⁵) and V_{L} (EDFATY⁸⁶) domains of ranibizumab (and corresponding sites in the Fab of bevacizumab). (See, e.g., Yang et al., 2015, Molecules 20:2138-2164, esp. at p. 2154 which is incorporated by reference in its entirety for the analysis of amino acids surrounding tyrosine residues subjected to protein tyrosine sulfation. The “rules” can be summarized as follows: Y residues with E or D within +5 to −5 position of Y, and where position −1 of Y is a neutral or acidic charged amino acid—but not a basic amino acid, e.g., R, K, or H that abolishes sulfation). Human IgG antibodies can manifest a number of other post-translational modifications, such as N-terminal modifications, C-terminal modifications, degradation or oxidation of amino acid residues, cysteine related variants, and glycation (See, e.g., Liu et al., 2014, mAbs 6(5):1145-1154).
(v) Glycosylation of anti-VEGF Fabs, such as ranibizumab or the Fab fragment of bevacizumab by human retinal cells will result in the addition of glycans that can improve stability, half-life and reduce unwanted aggregation and/or immunogenicity of the transgene product. (See, e.g., Bovenkamp et al., 2016, J. Immunol. 196: 1435-1441 for a review of the emerging importance of Fab glycosylation). Significantly, glycans that can be added to HuPTMFabVEGFi, e.g., HuGlyFabVEGFi, provided herein, are highly processed complex-type biantennary N-glycans that contain 2,6-sialic acid (e.g., see FIG. 2 depicting the glycans that may be incorporated into HuPTMFabVEGFi, e.g., HuGlyFabVEGFi) and bisecting GlcNAc, but not NGNA. Such glycans are not present in ranibizumab (which is made in E. coli and is not glycosylated at all) or in bevacizumab (which is made in CHO cells that do not have the 2,6-sialyltransferase required to make this post-translational modification, nor do CHO cells product bisecting GlcNAc, although they do produce NGNA, which is immunogenic). See, e.g., Dumont et al., 2015, Crit. Rev. Biotechnol. (Early Online, published online September 18, 2015, pp. 1-13 at p. 5). The human glycosylation pattern of the HuPTMFabVEGFi, e.g., HuGlyFabVEGFi, provided herein, should reduce immunogenicity of the transgene product and improve efficacy.
(vi) Tyrosine-sulfation of anti-VEGF Fabs, such as ranibizumab or the Fab fragment of bevacizumab—a robust post-translational process in human retinal cells—could result in transgene products with increased avidity for VEGF. Indeed, tyrosine-sulfation of the Fab of therapeutic antibodies against other targets has been shown to dramatically increase avidity for antigen and activity. (See, e.g., Loos et al., 2015, PNAS 112: 12675-12680, and Choe et al., 2003, Cell 114: 161-170). Such post-translational modifications are not present on ranibizumab (which is made in E. coli a host that does not possess the enzymes required for tyrosine-sulfation), and at best is under-represented in bevacizumab—a CHO cell product. Unlike human retinal cells, CHO cells are not secretory cells and have a limited capacity for post-translational tyrosine-sulfation. (See, e.g., Mikkelsen & Ezban, 1991, Biochemistry 30: 1533-1537, esp. discussion at p. 1537).

For the foregoing reasons, the production of HuPTMFabVEGFi, e.g., HuGlyFabVEGFi, should result in a “biobetter” molecule for the treatment of nAMD accomplished via gene therapy—e.g., by administering a viral vector or other DNA expression construct encoding HuPTMFabVEGFi, e.g., HuGlyFabVEGFi, to the subretinal space in the eye(s) of patients (human subjects) diagnosed with nAMD, to create a permanent depot in the eye that continuously supplies the fully-human post-translationally modified, e.g., human-glycosylated, sulfated transgene product produced by transduced retinal cells. The cDNA construct for the FabVEGFi should include a signal peptide that ensures proper co- and post-translational processing (glycosylation and protein sulfation) by the transduced retinal cells. Such signal sequences used by retinal cells may include but are not limited to:

MNFLLSWVHW SLALLLYLHH AKWSQA
(VEGF-A signal peptide)
MERAAPSRRV PLPLLLLGGL ALLAAGVDA
(Fibulin-1 signal peptide)
MAPLRPLLIL ALLAWVALA
(Vitronectin signal peptide)
MRLLAKIICLMLWAICVA
(Complement Factor H signal peptide)
MRLLAFLSLL ALVLQETGT
(Opticin signal peptide)
MKWVTFISLLFLFSSAYS
(Albumin signal peptide)
MAFLWLLSCWALLGTTFG
(Chymotrypsinogen signal peptide)
MYRMQLLSCIALILALVTNS
(Interleukin-2 signal peptide)
MNLLLILTFVAAAVA
(Trypsinogen-2 signal peptide).

See, e.g., Stern et al., 2007, Trends Cell. Mol. Biol., 2:1-17 and Dalton & Barton, 2014, Protein Sci, 23: 517-525, each of which is incorporated by reference herein in its entirety for the signal peptides that can be used.

As an alternative, or an additional treatment to gene therapy, the HuPTMFabVEGFi product, e.g., HuGlyFabVEGFi glycoprotein, can be produced in human cell lines by recombinant DNA technology, and administered to patients diagnosed with nAMD by intravitreal injection. The HuPTMFabVEGFi product, e.g., glycoprotein, may also be administered to patients with AMD or diabetic retinopathy. Human cell lines that can be used for such recombinant glycoprotein production include but are not limited to human embryonic kidney 293 cells (HEK293), fibrosarcoma HT-1080, HKB-11, CAP, HuH-7, and retinal cell lines, PER.C6, or RPE to name a few (e.g., see Dumont et al., 2015, Crit. Rev. Biotechnol. (Early Online, published online September 18, 2015, pp. 1-13) “Human cell lines for biopharmaceutical manufacturing: history, status, and future perspectives” which is incorporated by reference in its entirety for a review of the human cell lines that could be used for the recombinant production of the HuPTMFabVEGFi product, e.g., HuGlyFabVEGFi glycoprotein). To ensure complete glycosylation, especially sialylation, and tyrosine-sulfation, the cell line used for production can be enhanced by engineering the host cells to co-express α-2,6-sialyltransferase (or both α-2,3- and α-2,6-sialyltransferases) and/or TPST-1 and TPST-2 enzymes responsible for tyrosine-O-sulfation in retinal cells.

Combinations of delivery of the HuPTMFabVEGFi, e.g., HuGlyFabVEGFi, to the eye/retina accompanied by delivery of other available treatments are encompassed by the methods provided herein. The additional treatments may be administered before, concurrently or subsequent to the gene therapy treatment. Available treatments for nAMD that could be combined with the gene therapy provided herein include but are not limited to laser photocoagulation, photodynamic therapy with verteporfin, and intravitreal (IVT) injections with anti-VEGF agents, including but not limited to pegaptanib, ranibizumab, aflibercept, or bevacizumab. Additional treatments with anti-VEGF agents, such as biologics, may be referred to as “rescue” therapy.

Unlike small molecule drugs, biologics usually comprise a mixture of many variants with different modifications or forms that have a different potency, pharmacokinetics, and safety profile. It is not essential that every molecule produced either in the gene therapy or protein therapy approach be fully glycosylated and sulfated. Rather, the population of glycoproteins produced should have sufficient glycosylation (from about 1% to about 10% of the population), including 2,6-sialylation, and sulfation to demonstrate efficacy. The goal of gene therapy treatment provided herein is to slow or arrest the progression of retinal degeneration, and to slow or prevent loss of vision with minimal intervention/invasive procedures. Efficacy may be monitored by measuring BCVA (Best-Corrected Visual Acuity), intraocular pressure, slit lamp biomicroscopy, indirect ophthalmoscopy, SD-OCT (SD-Optical Coherence Tomography), electroretinography (ERG). Signs of vision loss, infection, inflammation and other safety events, including retinal detachment may also be monitored. Retinal thickness may be monitored to determine efficacy of the treatments provided herein. Without being bound by any particular theory, thickness of the retina may be used as a clinical readout, wherein the greater reduction in retinal thickness or the longer period of time before thickening of the retina, the more efficacious the treatment. Retinal thickness may be determined, for example, by SD-OCT. SD-OCT is a three-dimensional imaging technology which uses low-coherence interferometry to determine the echo time delay and magnitude of backscattered light reflected off an object of interest. OCT can be used to scan the layers of a tissue sample (e.g., the retina) with 3 to 15 μm axial resolution, and SD-OCT improves axial resolution and scan speed over previous forms of the technology (Schuman, 2008, Trans. Am. Opthamol. Soc. 106:426-458). Retinal function may be determined, for example, by ERG. ERG is a non-invasive electrophysiologic test of retinal function, approved by the FDA for use in humans, which examines the light sensitive cells of the eye (the rods and cones), and their connecting ganglion cells, in particular, their response to a flash stimulation.

### 5.1 N-Glycosylation, Tyrosine Sulfation, and O-Glycosylation

The amino acid sequence (primary sequence) of the anti-VEGF antigen-binding fragment of a HuPTMFabVEGFi, e.g., HuGlyFabVEGFi, used in the methods described herein comprises at least one site at which N-glycosylation or tyrosine sulfation takes place. In certain embodiments, the amino acid sequence of the anti-VEGF antigen-binding fragment comprises at least one N-glycosylation site and at least one tyrosine sulfation site. Such sites are described in detail below. In certain embodiments, the amino acid sequence of the anti-VEGF antigen-binding fragment comprises at least one O-glycosylation site, which can be in addition to one or more N-glycosylation sites and/or tyrosine sulfation sites present in said amino acid sequence.

5.1.1 N-Glycosylation

Reverse Glycosylation Sites

The canonical N-glycosylation sequence is known in the art to be Asn-X-Ser(or Thr), wherein X can be any amino acid except Pro. However, it recently has been demonstrated that asparagine (Asn) residues of human antibodies can be glycosylated in the context of a reverse consensus motif, Ser(or Thr)-X-Asn, wherein X can be any amino acid except Pro. See Valliere-Douglass et al., 2009, J. Biol. Chem. 284:32493-32506; and Valliere-Douglass et al., 2010, J. Biol. Chem. 285:16012-16022. As disclosed herein, and contrary to the state of the art understanding, anti-VEGF antigen-binding fragments for use in accordance with the methods described herein, e.g., ranibizumab, comprise several of such reverse consensus sequences. Accordingly, the methods described herein comprise use of anti-VEGF antigen-binding fragments that comprise at least one N-glycosylation site comprising the sequence Ser(or Thr)-X-Asn, wherein X can be any amino acid except Pro (also referred to herein as a “reverse N-glycosylation site”).

In certain embodiments, the methods described herein comprise use of an anti-VEGF antigen-binding fragment that comprises one, two, three, four, five, six, seven, eight, nine, ten, or more than ten N-glycosylation sites comprising the sequence Ser(or Thr)-X-Asn, wherein X can be any amino acid except Pro. In certain embodiments, the methods described herein comprise use of an anti-VEGF antigen-binding fragment that comprises one, two, three, four, five, six, seven, eight, nine, ten, or more than ten reverse N-glycosylation sites, as well as one, two, three, four, five, six, seven, eight, nine, ten, or more than ten non-consensus N-glycosylation sites (as defined herein, below).

In a specific embodiment, the anti-VEGF antigen-binding fragment comprising one or more reverse N-glycosylation sites used in the methods described herein is ranibizumab, comprising a light chain and a heavy chain of SEQ ID NOs. 1 and 2, respectively. In another specific embodiment, the anti-VEGF antigen-binding fragment comprising one or more reverse N-glycosylation sites used in the methods comprises the Fab of bevacizumab, comprising a light chain and a heavy chain of SEQ ID NOs. 3 and 4, respectively.

Non-Consensus Glycosylation Sites

In addition to reverse N-glycosylation sites, it recently has been demonstrated that glutamine (Gln) residues of human antibodies can be glycosylated in the context of a non-consensus motif, Gln-Gly-Thr. See Valliere-Douglass et al., 2010, J. Biol. Chem. 285:16012-16022. Surprisingly, anti-VEGF antigen-binding fragments for use in accordance with the methods described herein, e.g., ranibizumab, comprise several of such non-consensus sequences. Accordingly, the methods described herein comprise use of anti-VEGF antigen-binding fragments that comprise at least one N-glycosylation site comprising the sequence Gln-Gly-Thr (also referred to herein as a “non-consensus N-glycosylation site”).

In certain embodiments, the methods described herein comprise use of an anti-VEGF antigen-binding fragment that comprises one, two, three, four, five, six, seven, eight, nine, ten, or more than ten N-glycosylation sites comprising the sequence Gln-Gly-Thr.

In a specific embodiment, the anti-VEGF antigen-binding fragment comprising one or more non-consensus N-glycosylation sites used in the methods described herein is ranibizumab (comprising a light chain and a heavy chain of SEQ ID NOs. 1 and 2, respectively). In another specific embodiment, the anti-VEGF antigen-binding fragment comprising one or more non-consensus N-glycosylation sites used in the methods comprises the Fab of bevacizumab (comprising a light chain and a heavy chain of SEQ ID NOs. 3 and 4, respectively).

Engineered N-Glycosylation Sites

In certain embodiments, a nucleic acid encoding an anti-VEGF antigen-binding fragment is modified to include 1, 2, 3, 4, 5, 6, 7, 8, 9, 10, or more N-glycosylation sites (including the canonical N-glycosylation consensus sequence, reverse N-glycosylation site, and non-consensus N-glycosylation sites) than would normally be associated with the HuGlyFabVEGFi (e.g., relative to the number of N-glycosylation sites associated with the anti-VEGF antigen-binding fragment in its unmodified state). In specific embodiments, introduction of glycosylation sites is accomplished by insertion of N-glycosylation sites (including the canonical N-glycosylation consensus sequence, reverse N-glycosylation site, and non-consensus N-glycosylation sites) anywhere in the primary structure of the antigen-binding fragment, so long as said introduction does not impact binding of the antigen-binding fragment to its antigen, VEGF. Introduction of glycosylation sites can be accomplished by, e.g., adding new amino acids to the primary structure of the antigen-binding fragment, or the antibody from which the antigen-binding fragment is derived (i.e., the glycosylation sites are added, in full or in part), or by mutating existing amino acids in the antigen-binding fragment, or the antibody from which the antigen-binding fragment is derived, in order to generate the N-glycosylation sites (i.e., amino acids are not added to the antigen-binding fragment/antibody, but selected amino acids of the antigen-binding fragment/antibody are mutated so as to form N-glycosylation sites). Those of skill in the art will recognize that the amino acid sequence of a protein can be readily modified using approaches known in the art, e.g., recombinant approaches that include modification of the nucleic acid sequence encoding the protein.

In a specific embodiment, an anti-VEGF antigen-binding fragment used in the method described herein is modified such that, when expressed in retinal cells, it can be hyperglycosylated. See Courtois et al., 2016, mAbs 8:99-112 which is incorporated by reference herein in its entirety. In a specific embodiment, said anti-VEGF antigen-binding fragment is ranibizumab (comprising a light chain and a heavy chain of SEQ ID NOs. 1 and 2, respectively). In another specific embodiment, said anti-VEGF antigen-binding fragment comprises the Fab of bevacizumab (comprising a light chain and a heavy chain of SEQ ID NOs. 3 and 4, respectively).

N-Glycosylation of Anti-VEGF Antigen-Binding Fragments

Unlike small molecule drugs, biologics usually comprise a mixture of many variants with different modifications or forms that have a different potency, pharmacokinetics, and safety profile. It is not essential that every molecule produced either in the gene therapy or protein therapy approach be fully glycosylated and sulfated. Rather, the population of glycoproteins produced should have sufficient glycosylation (including 2,6-sialylation) and sulfation to demonstrate efficacy. The goal of gene therapy treatment provided herein is to slow or arrest the progression of retinal degeneration, and to slow or prevent loss of vision with minimal intervention/invasive procedures.

In a specific embodiment, an anti-VEGF antigen-binding fragment, e.g., ranibizumab, used in accordance with the methods described herein, when expressed in a retinal cell, could be glycosylated at 100% of its N-glycosylation sites. However, one of skill in the art will appreciate that not every N-glycosylation site of an anti-VEGF antigen-binding fragment need be N-glycosylated in order for benefits of glycosylation to be attained. Rather, benefits of glycosylation can be realized when only a percentage of N-glycosylation sites are glycosylated, and/or when only a percentage of expressed antigen-binding fragments are glycosylated. Accordingly, in certain embodiments, an anti-VEGF antigen-binding fragment used in accordance with the methods described herein, when expressed in a retinal cell, is glycosylated at 10%-20%, 20%-30%, 30%-40%, 40%-50%, 50%-60%, 60%-70%, 70%-80%, 80%-90%, or 90%-100% of it available N-glycosylation sites. In certain embodiments, when expressed in a retinal cell, 10%-20%, 20%-30%, 30%-40%, 40%-50%, 50%-60%, 60%-70%, 70%-80%, 80%-90%, or 90%-100% of the an anti-VEGF antigen-binding fragments used in accordance with the methods described herein are glycosylated at least one of their available N-glycosylation sites.

In a specific embodiment, at least 10%, 20% 30%, 40%, 50%, 60%, 70%, 75%, 80%, 85%, 90%, 95%, or 99% of the N-glycosylation sites present in an anti-VEGF antigen-binding fragment used in accordance with the methods described herein are glycosylated at an Asn residue (or other relevant residue) present in an N-glycosylation site, when the anti-VEGF antigen-binding fragment is expressed in a retinal cell. That is, at least 50%, 60%, 70%, 75%, 80%, 85%, 90%, 95%, or 99% of the N-glycosylation sites of the resultant HuGlyFabVEGFi are glycosylated.

In another specific embodiment, at least 10%, 20% 30%, 40%, 50%, 60%, 70%, 75%, 80%, 85%, 90%, 95%, or 99% of the N-glycosylation sites present in an anti-VEGF antigen-binding fragment used in accordance with the methods described herein are glycosylated with an identical attached glycan linked to the Asn residue (or other relevant residue) present in an N-glycosylation site, when the anti-VEGF antigen-binding fragment is expressed in a retinal cell. That is, at least 50%, 60%, 70%, 75%, 80%, 85%, 90%, 95%, or 99% of the N-glycosylation sites of the resultant HuGlyFabVEGFi an identical attached glycan.

When an anti-VEGF antigen-binding fragment, e.g., ranibizumab, used in accordance with the methods described herein is expressed in a retinal cell, the N-glycosylation sites of the of the antigen-binding fragment can be glycosylated with various different glycans. N-glycans of antigen-binding fragments have been characterized in the art. For example, Bondt et al., 2014, Mol. & Cell. Proteomics 13.11:3029-3039 (incorporated by reference herein in its entirety for it disclosure of Fab-associated N-glycans) characterizes glycans associated with Fabs, and demonstrates that Fab and Fc portions of antibodies comprise distinct glycosylation patterns, with Fab glycans being high in galactosylation, sialylation, and bisection (e.g., with bisecting GlcNAc) but low in fucosylation with respect to Fc glycans. Like Bondt, Huang et al., 2006, Anal. Biochem. 349:197-207 (incorporated by reference herein in its entirety for it disclosure of Fab-associated N-glycans) found that most glycans of Fabs are sialylated. However, in the Fab of the antibody examined by Huang (which was produced in a murine cell background), the identified sialic residues were N-Glycolylneuraminic acid (“Neu5Gc” or “NeuGc”) (which is not natural to humans) instead of N-acetylneuraminic acid (“Neu5Ac,” the predominant human sialic acid). In addition, Song et al., 2014, Anal. Chem. 86:5661-5666 (incorporated by reference herein in its entirety for it disclosure of Fab-associated N-glycans) describes a library of N-glycans associated with commercially available antibodies.

Importantly, when the anti-VEGF antigen-binding fragments, e.g., ranibizumab, used in accordance with the methods described herein are expressed in human retinal cells, the need for in vitro production in prokaryotic host cells (e.g., E. coli) or eukaryotic host cells (e.g., CHO cells) is circumvented. Instead, as a result of the methods described herein (e.g., use of retinal cells to express anti-hVEGF antigen-binding fragments), N-glycosylation sites of the anti-VEGF antigen-binding fragments are advantageously decorated with glycans relevant to and beneficial to treatment of humans. Such an advantage is unattainable when CHO cells or E. coli are utilized in antibody/antigen-binding fragment production, because e.g., CHO cells (1) do not express 2,6 sialyltransferase and thus cannot add 2,6 sialic acid during N-glycosylation and (2) can add Neu5Gc as sialic acid instead of Neu5Ac; and because E. coli does not naturally contain components needed for N-glycosylation. Accordingly, in one embodiment, an anti-VEGF antigen-binding fragment expressed in a retinal cell to give rise to a HuGlyFabVEGFi used in the methods of treatment described herein is glycosylated in the manner in which a protein is N-glycosylated in human retinal cells, e.g., retinal pigment cells, but is not glycosylated in the manner in which proteins are glycosylated in CHO cells. In another embodiment, an anti-VEGF antigen-binding fragment expressed in a retinal cell to give rise to a HuGlyFabVEGFi used in the methods of treatment described herein is glycosylated in the manner in which a protein is N-glycosylated in human retinal cells, e.g., retinal pigment cells, wherein such glycosylation is not naturally possible using a prokaryotic host cell, e.g., using E. coli.

In certain embodiments, a HuGlyFabVEGFi, e.g., ranibizumab, used in accordance with the methods described herein comprises one, two, three, four, five or more distinct N-glycans associated with Fabs of human antibodies. In a specific embodiment, said N-glycans associated with Fabs of human antibodies are those described in Bondt et al., 2014, Mol. & Cell. Proteomics 13.11:3029-3039, Huang et al., 2006, Anal. Biochem. 349:197-207, and/or Song et al., 2014, Anal. Chem. 86:5661-5666. In certain embodiments, a HuGlyFabVEGFi, e.g., ranibizumab, used in accordance with the methods described herein does not comprise NeuGc.

In a specific embodiment, the HuGlyFabVEGFi, e.g., ranibizumab, used in accordance with the methods described herein are predominantly glycosylated with a glycan comprising 2,6-linked sialic acid. In certain embodiments, HuGlyFabVEGFi comprising 2,6-linked sialic acid is polysialylated, i.e., contains more than one sialic acid. In certain embodiments, each N-glycosylation site of said HuGlyFabVEGFi comprises a glycan comprising 2,6-linked sialic acid, i.e., 100% of the N-glycosylation site of said HuGlyFabVEGFi comprise a glycan comprising 2,6-linked sialic acid. In another specific embodiment, at least 20%, 30%, 40%, 50%, 60%, 70%, 75%, 80%, 85%, 90%, 95%, or 99% of the N-glycosylation sites of a HuGlyFabVEGFi used in accordance with the methods described herein are glycosylated with a glycan comprising 2,6-linked sialic acid. In another specific embodiment, at least 10%-20%, 20%-30%, 30%-40%, 40%-50%, 50%-60%, 60%-70%, 70%-80%, 80%-90%, or 90%-99% of the N-glycosylation sites of a HuGlyFabVEGFi used in accordance with the methods described herein are glycosylated with a glycan comprising 2,6-linked sialic acid. In another specific embodiment, at least 20%, 30%, 40%, 50%, 60%, 70%, 75%, 80%, 85%, 90%, 95%, or 99% of the antigen-binding fragments expressed in a retinal cell in accordance with methods described herein (i.e., the antigen-binding fragments that give rise to HuGlyFabVEGFi, e.g., ranibizumab) are glycosylated with a glycan comprising 2,6-linked sialic acid. In another specific embodiment, at least 10%-20%, 20%-30%, 30%-40%, 40%-50%, 50%-60%, 60%-70%, 70%-80%, 80%-90%, or 90%-99% of the antigen-binding fragments expressed in a retinal cell in accordance with methods described herein (i.e., the Fabs that give rise to HuGlyFabVEGFi, e.g., ranibizumab) are glycosylated with a glycan comprising 2,6-linked sialic acid. In another specific embodiment, said sialic acid is Neu5Ac. In accordance with such embodiments, when only a percentage of the N-glycosylation sites of a HuGlyFabVEGFi are 2,6 sialylated or polysialylated, the remaining N-glycosylation can comprise a distinct N-glycan, or no N-glycan at all (i.e., remain non-glycosylated).

When a HuGlyFabVEGFi is 2,6 polysialylated, it comprises multiple sialic acid residues, e.g., 2, 3, 4, 5, 6, 7, 8, 9, 10 or more than 10 sialic acid residues. In certain embodiments, when a HuGlyFabVEGFi is polysialylated, it comprises 2-5, 5-10, 10-20, 20-30, 30-40, or 40-50 sialic acid residues. In certain embodiments, when a HuGlyFabVEGFi is polysialylated, it comprises 2,6-linked (sialic acid)ⁿ, wherein n can be any number from 1-100.

In a specific embodiment, the HuGlyFabVEGFi, e.g., ranibizumab, used in accordance with the methods described herein are predominantly glycosylated with a glycan comprising a bisecting GlcNAc. In certain embodiments, each N-glycosylation site of said HuGlyFabVEGFi comprises a glycan comprising a bisecting GlcNAc, i.e., 100% of the N-glycosylation site of said HuGlyFabVEGFi comprise a glycan comprising a bisecting GlcNAc. In another specific embodiment, at least 20%, 30%, 40%, 50%, 60%, 70%, 75%, 80%, 85%, 90%, 95%, or 99% of the N-glycosylation sites of a HuGlyFabVEGFi used in accordance with the methods described herein are glycosylated with a glycan comprising a bisecting GlcNAc. In another specific embodiment, at least 10%-20%, 20%-30%, 30%-40%, 40%-50%, 50%-60%, 60%-70%, 70%-80%, 80%-90%, or 90%-99% of the N-glycosylation sites of a HuGlyFabVEGFi used in accordance with the methods described herein are glycosylated with a glycan comprising a bisecting GlcNAc. In another specific embodiment, at least 20%, 30%, 40%, 50%, 60%, 70%, 75%, 80%, 85%, 90%, 95%, or 99% of the antigen-binding fragments expressed in a retinal cell in accordance with methods described herein (i.e., the antigen-binding fragments that give rise to HuGlyFabVEGFi, e.g., ranibizumab) are glycosylated with a glycan comprising a bisecting GlcNAc. In another specific embodiment, at least 10%-20%, 20%-30%, 30%-40%, 40%-50%, 50%-60%, 60%-70%, 70%-80%, 80%-90%, or 90%-99% of the antigen-binding fragments expressed in a retinal cell in accordance with methods described herein (i.e., the antigen-binding fragments that give rise to HuGlyFabVEGFi, e.g., ranibizumab) are glycosylated with a glycan comprising a bisecting GlcNAc.

In certain embodiments, the HuGlyFabVEGFi, e.g., ranibizumab, used in accordance with the methods described herein are hyperglycosylated, i.e., in addition to the N-glycosylation resultant from the naturally occurring N-glycosylation sites, said HuGlyFabVEGFi comprise glycans at N-glycosylation sites engineered to be present in the amino acid sequence of the antigen-binding fragment giving rise to HuGlyFabVEGFi. In certain embodiments, the HuGlyFabVEGFi, e.g., ranibizumab, used in accordance with the methods described herein is hyperglycosylated but does not comprise NeuGc.

Assays for determining the glycosylation pattern of antibodies, including antigen-binding fragments are known in the art. For example, hydrazinolysis can be used to analyze glycans. First, polysaccharides are released from their associated protein by incubation with hydrazine (the Ludger Liberate Hydrazinolysis Glycan Release Kit, Oxfordshire, UK can be used). The nucleophile hydrazine attacks the glycosidic bond between the polysaccharide and the carrier protein and allows release of the attached glycans. N-acetyl groups are lost during this treatment and have to be reconstituted by re-N-acetylation. Glycans may also be released using enzymes such as glycosidases or endoglycosidases, such as PNGase F and Endo H, which cleave cleanly and with fewer side reactions than hydrazines. The free glycans can be purified on carbon columns and subsequently labeled at the reducing end with the fluorophor 2-amino benzamide. The labeled polysaccharides can be separated on a GlycoSep-N column (GL Sciences) according to the HPLC protocol of Royle et al, Anal Biochem 2002, 304(1):70-90. The resulting fluorescence chromatogram indicates the polysaccharide length and number of repeating units. Structural information can be gathered by collecting individual peaks and subsequently performing MS/MS analysis. Thereby the monosaccharide composition and sequence of the repeating unit can be confirmed and additionally in homogeneity of the polysaccharide composition can be identified. Specific peaks of low or high molecular weight can be analyzed by MALDI-MS/MS and the result used to confirm the glycan sequence. Each peak in the chromatogram corresponds to a polymer, e.g., glycan, consisting of a certain number of repeat units and fragments, e.g., sugar residues, thereof. The chromatogram thus allows measurement of the polymer, e.g., glycan, length distribution. The elution time is an indication for polymer length, while fluorescence intensity correlates with molar abundance for the respective polymer, e.g., glycan. Other methods for assessing glycans associated with antigen-binding fragments include those described by Bondt et al., 2014, Mol. & Cell. Proteomics 13.11:3029-3039, Huang et al., 2006, Anal. Biochem. 349:197-207, and/or Song et al., 2014, Anal. Chem. 86:5661-5666.

Homogeneity or heterogeneity of the glycan patterns associated with antibodies (including antigen-binding fragments), as it relates to both glycan length or size and numbers glycans present across glycosylation sites, can be assessed using methods known in the art, e.g., methods that measure glycan length or size and hydrodynamic radius. HPLC, such as Size exclusion, normal phase, reversed phase, and anion exchange HPLC, as well as capillary electrophoresis, allows the measurement of the hydrodynamic radius. Higher numbers of glycosylation sites in a protein lead to higher variation in hydrodynamic radius compared to a carrier with less glycosylation sites. However, when single glycan chains are analyzed, they may be more homogenous due to the more controlled length. Glycan length can be measured by hydrazinolysis, SDS PAGE, and capillary gel electrophoresis. In addition, homogeneity can also mean that certain glycosylation site usage patterns change to a broader/narrower range. These factors can be measured by Glycopeptide LC-MS/MS.

Benefits of N-Glycosylation

N-glycosylation confers numerous benefits on the HuGlyFabVEGFi used in the methods described herein. Such benefits are unattainable by production of antigen-binding fragments in E. coli, because E. coli does not naturally possess components needed for N-glycosylation. Further, some benefits are unattainable through antibody production in, e.g., CHO cells, because CHO cells lack components needed for addition of certain glycans (e.g., 2,6 sialic acid and bisecting GlcNAc) and because CHO cells can add glycans, e.g., Neu5Gc not typical to humans. See, e.g., Song et al., 2014, Anal. Chem. 86:5661-5666. Accordingly, by virtue of the discovery set forth herein that anti-VEGF antigen-binding fragments, e.g., ranibizumab, comprise non-canonical N-glycosylation sites (including both reverse and non-consensus glycosylation sites), a method of expressing such anti-VEGF antigen-binding fragments in a manner that results in their glycosylation (and thus improved benefits associated with the antigen-binding fragments) has been realized. In particular, expression of anti-VEGF antigen-binding fragments in human retinal cells results in the production of HuGlyFabVEGFi (e.g., ranibizumab) comprising beneficial glycans that otherwise would not be associated with the antigen-binding fragments or their parent antibody.

While non-canonical glycosylation sites usually result in low level glycosylation (e.g., 1-5%) of the antibody population, the functional benefits may be significant in immunoprivileged organs, such as the eye (See, e.g., van de Bovenkamp et al., 2016, J. Immunol. 196:1435-1441). For example, Fab glycosylation may affect the stability, half-life, and binding characteristics of an antibody. To determine the effects of Fab glycosylation on the affinity of the antibody for its target, any technique known to one of skill in the art may be used, for example, enzyme linked immunosorbent assay (ELISA), or surface plasmon resonance (SPR). To determine the effects of Fab glycosylation on the half-life of the antibody, any technique known to one of skill in the art may be used, for example, by measurement of the levels of radioactivity in the blood or organs (e.g., the eye) in a subject to whom a radiolabelled antibody has been administered. To determine the effects of Fab glycosylation on the stability, for example, levels of aggregation or protein unfolding, of the antibody, any technique known to one of skill in the art may be used, for example, differential scanning calorimetry (DSC), high performance liquid chromatography (HPLC), e.g., size exclusion high performance liquid chromatography (SEC-HPLC), capillary electrophoresis, mass spectrometry, or turbidity measurement. Provided herein, the HuGlyFabVEGFi transgene results in production of an antigen-binding fragment which is 0.5%, 1%, 2%, 3%, 4%, 5%, 6%, 7%, 8%, 9%, or 10% or more glycosylated at non-canonical sites. In certain embodiments, 0.5%, 1%, 2%, 3%, 4%, 5%, 6%, 7%, 8%, 9%, or 10% or more antigen-binding fragments from a population of antigen-binding fragments are glycosylated at non-canonical sites. In certain embodiments, 0.5%, 1%, 2%, 3%, 4%, 5%, 6%, 7%, 8%, 9%, or 10% or more non-canonical sites are glycosylated. In certain embodiments, the glycosylation of the antigen-binding fragment at these non-canonical sites is 25%, 50%, 100%, 200%, 300%, 400%, 500%, or more greater than the amount of glycosylation of these non-canonical sites in an antigen-binding fragment produced in HEK293 cells.

The presence of sialic acid on HuGlyFabVEGFi used in the methods described herein can impact clearance rate of the HuGlyFabVEGFi, e.g., the rate of clearance from the vitreous humour. Accordingly, sialic acid patterns of a HuGlyFabVEGFi can be used to generate a therapeutic having an optimized clearance rate. Method of assessing antigen-binding fragment clearance rate are known in the art. See, e.g., Huang et al., 2006, Anal. Biochem. 349:197-207.

In another specific embodiment, a benefit conferred by N-glycosylation is reduced aggregation. Occupied N-glycosylation sites can mask aggregation prone amino acid residues, resulting in decreased aggregation. Such N-glycosylation sites can be native to an antigen-binding fragment used herein, or engineered into an antigen-binding fragment used herein, resulting in HuGlyFabVEGFi that is less prone to aggregation when expressed, e.g., expressed in retinal cells. Methods of assessing aggregation of antibodies are known in the art. See, e.g., Courtois et al., 2016, mAbs 8:99-112 which is incorporated by reference herein in its entirety.

In another specific embodiment, a benefit conferred by N-glycosylation is reduced immunogenicity. Such N-glycosylation sites can be native to an antigen-binding fragment used herein, or engineered into an antigen-binding fragment used herein, resulting in HuGlyFabVEGFi that is less prone to immunogenicity when expressed, e.g., expressed in retinal cells.

In another specific embodiment, a benefit conferred by N-glycosylation is protein stability. N-glycosylation of proteins is well-known to confer stability on them, and methods of assessing protein stability resulting from N-glycosylation are known in the art. See, e.g., Sola and Griebenow, 2009, J Pharm Sci., 98(4): 1223-1245.

In another specific embodiment, a benefit conferred by N-glycosylation is altered binding affinity. It is known in the art that the presence of N-glycosylation sites in the variable domains of an antibody can increase the affinity of the antibody for its antigen. See, e.g., Bovenkamp et al., 2016, J. Immunol. 196:1435-1441. Assays for measuring antibody binding affinity are known in the art. See, e.g., Wright et al., 1991, EMBO J. 10:2717-2723; and Leibiger et al., 1999, Biochem. J. 338:529-538.

5.1.2 Tyrosine Sulfation

Tyrosine sulfation occurs at tyrosine (Y) residues with glutamate (E) or aspartate (D) within +5 to −5 position of Y, and where position −1 of Y is a neutral or acidic charged amino acid, but not a basic amino acid, e.g., arginine (R), lysine (K), or histidine (H) that abolishes sulfation. Surprisingly, anti-VEGF antigen-binding fragments for use in accordance with the methods described herein, e.g., ranibizumab, comprise tyrosine sulfation sites (see FIG. 1). Accordingly, the methods described herein comprise use of anti-VEGF antigen-binding fragments, e.g., HuPTMFabVEGFi, that comprise at least one tyrosine sulfation site, such the anti-VEGF antigen-binding fragments, when expressed in retinal cells, can be tyrosine sulfated.

Importantly, tyrosine-sulfated antigen-binding fragments, e.g., ranibizumab, cannot be produced in E. coli, which naturally does not possess the enzymes required for tyrosine-sulfation. Further, CHO cells are deficient for tyrosine sulfation—they are not secretory cells and have a limited capacity for post-translational tyrosine-sulfation. See, e.g., Mikkelsen & Ezban, 1991, Biochemistry 30: 1533-1537. Advantageously, the methods provided herein call for expression of anti-VEGF antigen-binding fragments, e.g., HuPTMFabVEGFi, for example, ranibizumab, in retinal cells, which are secretory and do have capacity for tyrosine sulfation. See Kanan et al., 2009, Exp. Eye Res. 89: 559-567 and Kanan & Al-Ubaidi, 2015, Exp. Eye Res. 133: 126-131 reporting the production of tyrosine-sulfated glycoproteins secreted by retinal cells.

Tyrosine sulfation is advantageous for several reasons. For example, tyrosine-sulfation of the antigen-binding fragment of therapeutic antibodies against targets has been shown to dramatically increase avidity for antigen and activity. See, e.g., Loos et al., 2015, PNAS 112: 12675-12680, and Choe et al., 2003, Cell 114: 161-170. Assays for detection tyrosine sulfation are known in the art. See, e.g., Yang et al., 2015, Molecules 20:2138-2164.

5.1.3 O-Glycosylation

O-glycosylation comprises the addition of N-acetyl-galactosamine to serine or threonine residues by the enzyme. It has been demonstrated that amino acid residues present in the hinge region of antibodies can be O-glycosylated. In certain embodiments, the anti-VEGF antigen-binding fragments, e.g., ranibizumab, used in accordance with the methods described herein comprise all or a portion of their hinge region, and thus are capable of being O-glycosylated when expressed in human retinal cells. The possibility of O-glycosylation confers another advantage to the HuPTMFabVEGFi, e.g., HuGlyFabVEGFi, provided herein, as compared to, e.g., antigen-binding fragments produced in E. coli, again because the E. coli naturally does not contain machinery equivalent to that used in human O-glycosylation. (Instead, O-glycosylation in E. coli has been demonstrated only when the bacteria is modified to contain specific O-glycosylation machinery. See, e.g., Faridmoayer et al., 2007, J. Bacteriol. 189:8088-8098.) O-glycosylated HuPTMFabVEGFi, e.g., HuGlyFabVEGFi, by virtue of possessing glycans, shares advantageous characteristics with N-glycosylated HuGlyFabVEGFi (as discussed above).

### 5.2 Constructs and Formulations

For use in the methods provided herein are viral vectors or other DNA expression constructs encoding an anti-VEGF antigen-binding fragment or a hyperglycosylated derivative of an anti-VEGF antigen-binding fragment. The viral vectors and other DNA expression constructs provided herein include any suitable method for delivery of a transgene to a target cell (e.g., retinal pigment epithelial cells). The means of delivery of a transgene include viral vectors, liposomes, other lipid-containing complexes, other macromolecular complexes, synthetic modified mRNA, unmodified mRNA, small molecules, non-biologically active molecules (e.g., gold particles), polymerized molecules (e.g., dendrimers), naked DNA, plasmids, phages, transposons, cosmids, or episomes. In some embodiments, the vector is a targeted vector, e.g., a vector targeted to retinal pigment epithelial cells.

In some aspects, the disclosure provides for a nucleic acid for use, wherein the nucleic acid encodes a HuPTMFabVEGFi, e.g., HuGlyFabVEGFi operatively linked to a promoter selected from the group consisting of: cytomegalovirus (CMV) promoter, Rous sarcoma virus (RSV) promoter, MMT promoter, EF-1 alpha promoter, UB6 promoter, chicken beta-actin promoter, CAG promoter, RPE65 promoter and opsin promoter.

In certain embodiments, provided herein are recombinant vectors that comprise one or more nucleic acids (e.g. polynucleotides). The nucleic acids may comprise DNA, RNA, or a combination of DNA and RNA. In certain embodiments, the DNA comprises one or more of the sequences selected from the group consisting of promoter sequences, the sequence of the gene of interest (the transgene, e.g., an anti-VEGF antigen-binding fragment), untranslated regions, and termination sequences. In certain embodiments, viral vectors provided herein comprise a promoter operably linked to the gene of interest.

In certain embodiments, nucleic acids (e.g., polynucleotides) and nucleic acid sequences disclosed herein may be codon-optimized, for example, via any codon-optimization technique known to one of skill in the art (see, e.g., review by Quax et al., 2015, Mol Cell 59:149-161).

In a specific embodiment, the constructs described herein comprise the following components: (1) AAV2 inverted terminal repeats that flank the expression cassette; (2) Control elements, which include a) the CB7 promoter, comprising the CMV enhancer/chicken β-actin promoter, b) a chicken β-actin intron and c) a rabbit β-globin poly A signal; and (3) nucleic acid sequences coding for the heavy and light chains of anti-VEGF antigen-binding fragment, separated by a self-cleaving furin (F)/F2A linker, ensuring expression of equal amounts of the heavy and the light chain polypeptides.

5.2.1 mRNA

In certain embodiments, the vectors provided herein are modified mRNA encoding for the gene of interest (e.g., the transgene, for example, an anti-VEGF antigen-binding fragment moiety). The synthesis of modified and unmodified mRNA for delivery of a transgene to retinal pigment epithelial cells is taught, for example, in Hansson et al., J. Biol. Chem., 2015, 290(9):5661-5672, which is incorporated by reference herein in its entirety. In certain embodiments, provided herein is a modified mRNA encoding for an anti-VEGF antigen-binding fragment moiety.

5.2.2 Viral Vectors

Viral vectors include adenovirus, adeno-associated virus (AAV, e.g., AAV8), lentivirus, helper-dependent adenovirus, herpes simplex virus, poxvirus, hemagglutinin virus of Japan (HVJ), alphavirus, vaccinia virus, and retrovirus vectors. Retroviral vectors include murine leukemia virus (MLV)- and human immunodeficiency virus (HIV)-based vectors. Alphavirus vectors include semliki forest virus (SFV) and sindbis virus (SIN). In certain embodiments, the viral vectors provided herein are recombinant viral vectors. In certain embodiments, the viral vectors provided herein are altered such that they are replication-deficient in humans. In certain embodiments, the viral vectors are hybrid vectors, e.g., an AAV vector placed into a “helpless” adenoviral vector. In certain embodiments, provided herein are viral vectors comprising a viral capsid from a first virus and viral envelope proteins from a second virus. In specific embodiments, the second virus is vesicular stomatitus virus (VSV). In more specific embodiments, the envelope protein is VSV-G protein.

In certain embodiments, the viral vectors provided herein are HIV based viral vectors. In certain embodiments, HIV-based vectors provided herein comprise at least two polynucleotides, wherein the gag and pol genes are from an HIV genome and the env gene is from another virus.

In certain embodiments, the viral vectors provided herein are herpes simplex virus-based viral vectors. In certain embodiments, herpes simplex virus-based vectors provided herein are modified such that they do not comprise one or more immediately early (IE) genes, rendering them non-cytotoxic.

In certain embodiments, the viral vectors provided herein are MLV based viral vectors. In certain embodiments, MLV-based vectors provided herein comprise up to 8 kb of heterologous DNA in place of the viral genes.

In certain embodiments, the viral vectors provided herein are lentivirus-based viral vectors. In certain embodiments, lentiviral vectors provided herein are derived from human lentiviruses. In certain embodiments, lentiviral vectors provided herein are derived from non-human lentiviruses. In certain embodiments, lentiviral vectors provided herein are packaged into a lentiviral capsid. In certain embodiments, lentiviral vectors provided herein comprise one or more of the following elements: long terminal repeats, a primer binding site, a polypurine tract, att sites, and an encapsidation site.

In certain embodiments, the viral vectors provided herein are alphavirus-based viral vectors. In certain embodiments, alphavirus vectors provided herein are recombinant, replication-defective alphaviruses. In certain embodiments, alphavirus replicons in the alphavirus vectors provided herein are targeted to specific cell types by displaying a functional heterologous ligand on their virion surface.

In certain embodiments, the viral vectors provided herein are AAV based viral vectors. In preferred embodiments, the viral vectors provided herein are AAV8 based viral vectors. In certain embodiments, the AAV8 based viral vectors provided herein retain tropism for retinal cells. In certain embodiments, the AAV-based vectors provided herein encode the AAV rep gene (required for replication) and/or the AAV cap gene (required for synthesis of the capsid proteins). Multiple AAV serotypes have been identified. In certain embodiments, AAV-based vectors provided herein comprise components from one or more serotypes of AAV. In certain embodiments, AAV based vectors provided herein comprise capsid components from one or more of AAV1, AAV2, AAV3, AAV4, AAV5, AAV6, AAV7, AAV8, AAV9, AAV10, AAV11, or AAVrh10. In preferred embodiments, AAV based vectors provided herein comprise components from one or more of AAV8, AAV9, AAV10, AAV11, or AAVrh10 serotypes.

In certain embodiments, the AAV that is used in the methods described herein is Anc80 or Anc80L65, as described in Zinn et al., 2015, Cell Rep. 12(6): 1056-1068, which is incorporated by reference in its entirety. In certain embodiments, the AAV that is used in the methods described herein comprises one of the following amino acid insertions: LGETTRP or LALGETTRP, as described in U.S. Pat. Nos. 9,193,956; 9458517; and 9,587,282 and US patent application publication no. 2016/0376323, each of which is incorporated herein by reference in its entirety. In certain embodiments, the AAV that is used in the methods described herein is AAV.7m8, as described in U.S. Pat. Nos. 9,193,956; 9,458,517; and 9,587,282 and US patent application publication no. 2016/0376323, each of which is incorporated herein by reference in its entirety. In certain embodiments, the AAV that is used in the methods described herein is any AAV disclosed in U.S. Pat. No. 9,585,971, such as AAV-PHP.B. In certain embodiments, the AAV that is used in the methods described herein is an AAV disclosed in any of the following patents and patent applications, each of which is incorporated herein by reference in its entirety: U.S. Pat. Nos. 7,906,111; 8,524,446; 8,999,678; 8,628,966; 8,927,514; 8,734,809; 9,284,357; 9,409,953; 9,169,299; 9,193,956; 9458517; and 9,587,282 US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

AAV8-based viral vectors are used in certain of the methods described herein. Nucleic acid sequences of AAV based viral vectors and methods of making recombinant AAV and AAV capsids are taught, for example, in U.S. Pat. Nos. 7,282,199 B2, 7,790,449 B2, 8,318,480 B2, 8,962,332 B2 and International Patent Application No. PCT/EP2014/076466, each of which is incorporated herein by reference in its entirety. In one aspect, provided herein are AAV (e.g., AAV8)-based viral vectors encoding a transgene (e.g., an anti-VEGF antigen-binding fragment). In specific embodiments, provided herein are AAV8-based viral vectors encoding an anti-VEGF antigen-binding fragment. In more specific embodiments, provided herein are AAV8-based viral vectors encoding ranibizumab.

In certain embodiments, a single-stranded AAV (ssAAV) may be used supra. In certain embodiments, a self-complementary vector, e.g., scAAV, may be used (see, e.g., Wu, 2007, Human Gene Therapy, 18(2):171-82, McCarty et al, 2001, Gene Therapy, Vol 8, Number 16, Pages 1248-1254; and U.S. Pat. Nos. 6,596,535; 7,125,717; and 7,456,683, each of which is incorporated herein by reference in its entirety).

In certain embodiments, the viral vectors used in the methods described herein are adenovirus based viral vectors. A recombinant adenovirus vector may be used to transfer in the anti-VEGF antigen-binding fragment. The recombinant adenovirus can be a first generation vector, with an E1 deletion, with or without an E3 deletion, and with the expression cassette inserted into either deleted region. The recombinant adenovirus can be a second generation vector, which contains full or partial deletions of the E2 and E4 regions. A helper-dependent adenovirus retains only the adenovirus inverted terminal repeats and the packaging signal (phi). The transgene is inserted between the packaging signal and the 3′ITR, with or without stuffer sequences to keep the genome close to wild-type size of approx. 36 kb. An exemplary protocol for production of adenoviral vectors may be found in Alba et al., 2005, “Gutless adenovirus: last generation adenovirus for gene therapy,” Gene Therapy 12:S18-S27, which is incorporated by reference herein in its entirety.

In certain embodiments, the viral vectors used in the methods described herein are lentivirus based viral vectors. A recombinant lentivirus vector may be used to transfer in the anti-VEGF antigen-binding fragment. Four plasmids are used to make the construct: Gag/pol sequence containing plasmid, Rev sequence containing plasmids, Envelope protein containing plasmid (i.e. VSV-G), and Cis plasmid with the packaging elements and the anti-VEGF antigen-binding fragment gene.

For lentiviral vector production, the four plasmids are co-transfected into cells (i.e., HEK293 based cells), whereby polyethylenimine or calcium phosphate can be used as transfection agents, among others. The lentivirus is then harvested in the supernatant (lentiviruses need to bud from the cells to be active, so no cell harvest needs/should be done). The supernatant is filtered (0.45 μm) and then magnesium chloride and benzonase added. Further downstream processes can vary widely, with using TFF and column chromatography being the most GMP compatible ones. Others use ultracentrifugation with/without column chromatography. Exemplary protocols for production of lentiviral vectors may be found in Lesch et al., 2011, “Production and purification of lentiviral vector generated in 293T suspension cells with baculoviral vectors,” Gene Therapy 18:531-538, and Ausubel et al., 2012, “Production of CGMP-Grade Lentiviral Vectors,” Bioprocess Int. 10(2):32-43, both of which are incorporated by reference herein in their entireties.

In a specific embodiment, a vector for use in the methods described herein is one that encodes an anti-VEGF antigen-binding fragment (e.g., ranibizumab) such that, upon introduction of the vector into a relevant cell (e.g., a retinal cell in vivo or in vitro), a glycosylated and or tyrosine sulfated variant of the anti-VEGF antigen-binding fragment is expressed by the cell. In a specific embodiment, the expressed anti-VEGF antigen-binding fragment comprises a glycosylation and/or tyrosine sulfation pattern as described in Section 5.1, above.

5.2.3 Promoters and Modifiers of Gene Expression

In certain embodiments, the vectors provided herein comprise components that modulate gene delivery or gene expression (e.g., “expression control elements”). In certain embodiments, the vectors provided herein comprise components that modulate gene expression. In certain embodiments, the vectors provided herein comprise components that influence binding or targeting to cells. In certain embodiments, the vectors provided herein comprise components that influence the localization of the polynucleotide (e.g., the transgene) within the cell after uptake. In certain embodiments, the vectors provided herein comprise components that can be used as detectable or selectable markers, e.g., to detect or select for cells that have taken up the polynucleotide.

In certain embodiments, the viral vectors provided herein comprise one or more promoters. In certain embodiments, the promoter is a constitutive promoter. In certain embodiments, the promoter is an inducible promoter. In certain embodiments the promoter is a hypoxia-inducible promoter. In certain embodiments, the promoter comprises a hypoxia-inducible factor (HIF) binding site. In certain embodiments, the promoter comprises a HIF-1α binding site. In certain embodiments, the promoter comprises a HIF-2α binding site. In certain embodiments, the HIF binding site comprises an RCGTG motif. For details regarding the location and sequence of HIF binding sites, see, e.g., Schödel, et al., Blood, 2011, 117(23):e207-e217, which is incorporated by reference herein in its entirety. In certain embodiments, the promoter comprises a binding site for a hypoxia induced transcription factor other than a HIF transcription factor. In certain embodiments, the viral vectors provided herein comprise one or more IRES sites that is preferentially translated in hypoxia. For teachings regarding hypoxia-inducible gene expression and the factors involved therein, see, e.g., Kenneth and Rocha, Biochem J., 2008, 414:19-29, which is incorporated by reference herein in its entirety.

In certain embodiments, the promoter is a CB7 promoter (see Dinculescu et al., 2005, Hum Gene Ther 16: 649-663, incorporated by reference herein in its entirety). In some embodiments, the CB7 promoter includes other expression control elements that enhance expression of the transgene driven by the vector. In certain embodiments, the other expression control elements include chicken β-actin intron and/or rabbit β-globin polA signal. In certain embodiments, the promoter comprises a TATA box. In certain embodiments, the promoter comprises one or more elements. In certain embodiments, the one or more promoter elements may be inverted or moved relative to one another. In certain embodiments, the elements of the promoter are positioned to function cooperatively. In certain embodiments, the elements of the promoter are positioned to function independently. In certain embodiments, the viral vectors provided herein comprise one or more promoters selected from the group consisting of the human CMV immediate early gene promoter, the SV40 early promoter, the Rous sarcoma virus (RS) long terminal repeat, and rat insulin promoter. In certain embodiments, the vectors provided herein comprise one or more long terminal repeat (LTR) promoters selected from the group consisting of AAV, MLV, MMTV, SV40, RSV, HIV-1, and HIV-2 LTRs. In certain embodiments, the vectors provided herein comprise one or more tissue specific promoters (e.g., a retinal pigment epithelial cell-specific promoter). In certain embodiments, the viral vectors provided herein comprise a RPE65 promoter. In certain embodiments, the vectors provided herein comprise a VMD2 promoter.

In certain embodiments, the viral vectors provided herein comprise one or more regulatory elements other than a promoter. In certain embodiments, the viral vectors provided herein comprise an enhancer. In certain embodiments, the viral vectors provided herein comprise a repressor. In certain embodiments, the viral vectors provided herein comprise an intron or a chimeric intron. In certain embodiments, the viral vectors provided herein comprise a polyadenylation sequence.

5.2.4 Signal Peptides

In certain embodiments, the vectors provided herein comprise components that modulate protein delivery. In certain embodiments, the viral vectors provided herein comprise one or more signal peptides. Signal peptides may also be referred to herein as “leader sequences” or “leader peptides”. In certain embodiments, the signal peptides allow for the transgene product (e.g., the anti-VEGF antigen-binding fragment moiety) to achieve the proper packaging (e.g. glycosylation) in the cell. In certain embodiments, the signal peptides allow for the transgene product (e.g., the anti-VEGF antigen-binding fragment moiety) to achieve the proper localization in the cell. In certain embodiments, the signal peptides allow for the transgene product (e.g., the anti-VEGF antigen-binding fragment moiety) to achieve secretion from the cell. Examples of signal peptides to be used in connection with the vectors and transgenes provided herein may be found in Table 1.

TABLE 1
Signal peptides for use with the vectors
provided herein.

| SEQ ID | | |
|---|---|---|
| NO. | Signal Peptide | Sequence |
| 5 | VEGF-A signal | MNFLLSWVHW SLALLLYLHH |
| | peptide | AKWSQA |
| 6 | Fibulin-1 signal | MERAAPSRRV PLPLLLLGGL |
| | peptide | ALLAAGVDA |
| 7 | Vitronectin signal | MAPLRPLLIL ALLAWVALA |
| | peptide | |
| 8 | Complement Factor | MRLLAKIICLMLWAICVA |
| | H signal peptide | |
| 9 | Opticin signal | MRLLAFLSLL ALVLQETGT |
| | peptide | |
| 22 | Albumin signal | MKWVTFISLLFLFSSAYS |
| | peptide | |
| 23 | Chymotrypsinogen | MAFLWLLSCWALLGTTFG |
| | signal peptide | |
| 24 | Interleukin-2 | MYRMQLLSCIALILALVTNS |
| | signal peptide | |
| 25 | Trypsinogen-2 | MNLLLILTFVAAAVA |
| | signal peptide | |

5.2.5 Polycistronic Messages—IRES and F2A linkers

Internal ribosome entry sites. A single construct can be engineered to encode both the heavy and light chains separated by a cleavable linker or IRES so that separate heavy and light chain polypeptides are expressed by the transduced cells. In certain embodiments, the viral vectors provided herein provide polycistronic (e.g., bicistronic) messages. For example, the viral construct can encode the heavy and light chains separated by an internal ribosome entry site (IRES) elements (for examples of the use of IRES elements to create bicistronic vectors see, e.g., Gurtu et al., 1996, Biochem. Biophys. Res. Comm. 229(1):295-8, which is herein incorporated by reference in its entirety). IRES elements bypass the ribosome scanning model and begin translation at internal sites. The use of IRES in AAV is described, for example, in Furling et al., 2001, Gene Ther 8(11): 854-73, which is herein incorporated by reference in its entirety. In certain embodiments, the bicistronic message is contained within a viral vector with a restraint on the size of the polynucleotide(s) therein. In certain embodiments, the bicistronic message is contained within an AAV virus-based vector (e.g., an AAV8-based vector).

Furin-F2A linkers. In other embodiments, the viral vectors provided herein encode the heavy and light chains separated by a cleavable linker such as the self-cleaving furin/F2A (F/F2A) linkers (Fang et al., 2005, Nature Biotechnology 23: 584-590, and Fang, 2007, Mol Ther 15: 1153-9, each of which is incorporated by reference herein in its entirety).

For example, a furin-F2A linker may be incorporated into an expression cassette to separate the heavy and light chain coding sequences, resulting in a construct with the structure:

Leader-Heavy chain-Furin site-F2A site-Leader-Light chain-PolyA.

The F2A site, with the amino acid sequence LLNFDLLKLAGDVESNPGP (SEQ ID NO: 26) is self-processing, resulting in “cleavage” between the final G and P amino acid residues. Additional linkers that could be used include but are not limited to:

T2A: (GSG)
(SEQ ID NO: 27)
E G R G S L L T C G D V E E N P G P;
P2A: (GSG)
(SEQ ID NO: 28)
A T N F S L L K Q A G D V E E N P G P;
E2A: (GSG)
(SEQ ID NO: 29)
Q C T N Y A L L K L A G D V E S N P G P;
F2A: (GSG)
(SEQ ID NO: 30)
V K Q T L N F D L L K L A G D V E S N P G P.

A peptide bond is skipped when the ribosome encounters the F2A sequence in the open reading frame, resulting in the termination of translation, or continued translation of the downstream sequence (the light chain). This self-processing sequence results in a string of additional amino acids at the end of the C-terminus of the heavy chain. However, such additional amino acids are then cleaved by host cell Furin at the furin sites, located immediately prior to the F2A site and after the heavy chain sequence, and further cleaved by carboxypeptidases. The resultant heavy chain may have one, two, three, or more additional amino acids included at the C-terminus, or it may not have such additional amino acids, depending on the sequence of the Furin linker used and the carboxypeptidase that cleaves the linker in vivo (See, e.g., Fang et al., 17 Apr. 2005, Nature Biotechnol. Advance Online Publication; Fang et al., 2007, Molecular Therapy 15(6):1153-1159; Luke, 2012, Innovations in Biotechnology, Ch. 8, 161-186). Furin linkers that may be used comprise a series of four basic amino acids, for example, RKRR, RRRR, RRKR, or RKKR. Once this linker is cleaved by a carboxypeptidase, additional amino acids may remain, such that an additional zero, one, two, three or four amino acids may remain on the C-terminus of the heavy chain, for example, R, RR, RK, RKR, RRR, RRK, RKK, RKRR, RRRR, RRKR, or RKKR. In certain embodiments, one the linker is cleaved by an carboxypeptidase, no additional amino acids remain. In certain embodiments, 5%, 10%, 15%, or 20% of the antibody, e.g., antigen-binding fragment, population produced by the constructs for use in the methods described herein has one, two, three, or four amino acids remaining on the C-terminus of the heavy chain after cleavage. In certain embodiments, the furin linker has the sequence R-X-K/R-R, such that the additional amino acids on the C-terminus of the heavy chain are R, RX, RXK, RXR, RXKR, or RXRR, where X is any amino acid, for example, alanine (A). In certain embodiments, no additional amino acids may remain on the C-terminus of the heavy chain.

In certain embodiments, an expression cassette described herein is contained within a viral vector with a restraint on the size of the polynucleotide(s) therein. In certain embodiments, the expression cassette is contained within an AAV virus-based vector (e.g., an AAV8-based vector).

5.2.6 Untranslated Regions

In certain embodiments, the viral vectors provided herein comprise one or more untranslated regions (UTRs), e.g., 3′ and/or 5′ UTRs. In certain embodiments, the UTRs are optimized for the desired level of protein expression. In certain embodiments, the UTRs are optimized for the mRNA half life of the transgene. In certain embodiments, the UTRs are optimized for the stability of the mRNA of the transgene. In certain embodiments, the UTRs are optimized for the secondary structure of the mRNA of the transgene.

5.2.7 Inverted Terminal Repeats

In certain embodiments, the viral vectors provided herein comprise one or more inverted terminal repeat (ITR) sequences. ITR sequences may be used for packaging the recombinant gene expression cassette into the virion of the viral vector. In certain embodiments, the ITR is from an AAV, e.g., AAV8 or AAV2 (see, e.g., Yan et al., 2005, J. Virol., 79(1):364-379; U.S. Pat. Nos. 7,282,199 B2, 7,790,449 B2, 8,318,480 B2, 8,962,332 B2 and International Patent Application No. PCT/EP2014/076466, each of which is incorporated herein by reference in its entirety).

5.2.8 Transgenes

The HuPTMabVEGFi, e.g., HuGlyFabVEGFi encoded by the transgene can include, but is not limited to an antigen-binding fragment of an antibody that binds to VEGF, such as bevacizumab; an anti-VEGF Fab moiety such as ranibizumab; or such bevacizumab or ranibizumab Fab moieties engineered to contain additional glycosylation sites on the Fab domain (e.g., see Courtois et al., 2016, mAbs 8: 99-112 which is incorporated by reference herein in its entirety for it description of derivatives of bevacizumab that are hyperglycosylated on the Fab domain of the full length antibody).

In certain embodiments, the vectors provided herein encode an anti-VEGF antigen-binding fragment transgene. In specific embodiments, the anti-VEGF antigen-binding fragment transgene is controlled by appropriate expression control elements for expression in retinal cells: In certain embodiments, the anti-VEGF antigen-binding fragment transgene comprises Bevacizumab Fab portion of the light and heavy chain cDNA sequences (SEQ ID NOs. 10 and 11, respectively). In certain embodiments, the anti-VEGF antigen-binding fragment transgene comprises Ranibizumab light and heavy chain cDNA sequences (SEQ ID NOs. 12 and 13, respectively). In certain embodiments, the anti-VEGF antigen-binding fragment transgene encodes a Bevacizumab Fab, comprising a light chain and a heavy chain of SEQ ID NOs: 3 and 4, respectively. In certain embodiments, the anti-VEGF antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 3. In certain embodiments, the anti-VEGF antigen-binding fragment transgene encodes an antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 4. In certain embodiments, the anti-VEGF antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 3 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 4. In certain embodiments, the anti-VEGF antigen-binding fragment transgene encodes a hyperglycosylated Ranibizumab, comprising a light chain and a heavy chain of SEQ ID NOs: 1 and 2, respectively. In certain embodiments, the anti-VEGF antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 1. In certain embodiments, the anti-VEGF antigen-binding fragment transgene encodes an antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 2. In certain embodiments, the anti-VEGF antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 1 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 2. In certain embodiments, the anti-VEGF antigen-binding fragment transgene encodes a hyperglycosylated Bevacizumab Fab, comprising a light chain and a heavy chain of SEQ ID NOs: 3 and 4, with one or more of the following mutations: L118N (heavy chain), E195N (light chain), or Q160N or Q160S (light chain). In certain embodiments, the anti-VEGF antigen-binding fragment transgene encodes a hyperglycosylated Ranibizumab, comprising a light chain and a heavy chain of SEQ ID NOs: 1 and 2, with one or more of the following mutations: L118N (heavy chain), E195N (light chain), or Q160N or Q160S (light chain). The sequences of the antigen-binding fragment transgene cDNAs may be found, for example, in Table 2. In certain embodiments, the sequence of the antigen-binding fragment transgene cDNAs is obtained by replacing the signal sequence of SEQ ID NOs: 10 and 11 or SEQ ID NOs: 12 and 13 with one or more signal sequences listed in Table 1.

In certain embodiments, the anti-VEGF antigen-binding fragment transgene encodes an antigen-binding fragment and comprises the nucleotide sequences of the six bevacizumab CDRs. In certain embodiments, the anti-VEGF antigen-binding fragment transgene encodes an antigen-binding fragment and comprises the nucleotide sequences of the six ranibizumab CDRs. In certain embodiments, the anti-VEGF antigen-binding fragment transgene encodes an antigen-binding fragment comprising a heavy chain variable region comprising heavy chain CDRs 1-3 of ranibizumab (SEQ ID NOs: 20, 18, and 21). In certain embodiments, the anti-VEGF antigen-binding fragment transgene encodes an antigen-binding fragment comprising a light chain variable region comprising light chain CDRs 1-3 of ranibizumab (SEQ ID NOs: 14-16). In certain embodiments, the anti-VEGF antigen-binding fragment transgene encodes an antigen-binding fragment comprising a heavy chain variable region comprising heavy chain CDRs 1-3 of bevacizumab (SEQ ID NOs: 17-19). In certain embodiments, the anti-VEGF antigen-binding fragment transgene encodes an antigen-binding fragment comprising a light chain variable region comprising light chain CDRs 1-3 of bevacizumab (SEQ ID NOs: 14-16). In certain embodiments, the anti-VEGF antigen-binding fragment transgene encodes an antigen-binding fragment comprising a heavy chain variable region comprising heavy chain CDRs 1-3 of ranibizumab (SEQ ID NOs: 20, 18, and 21) and a light chain variable region comprising light chain CDRs 1-3 of ranibizumab (SEQ ID NOs: 14-16). In certain embodiments, the anti-VEGF antigen-binding fragment transgene encodes an antigen-binding fragment comprising a heavy chain variable region comprising heavy chain CDRs 1-3 of bevacizumab (SEQ ID NOs: 17-19) and a light chain variable region comprising light chain CDRs 1-3 of bevacizumab (SEQ ID NOs: 14-16).

TABLE 2
Exemplary transgene sequences

| VEGF antigen- | |
|---|---|
| binding fragment | |
| (SEQ ID NO.) | Sequence |
| Bevacizumab cDNA | gctagcgcca ccatgggctg gtcctgcatc atcctgttcc tggtggccac |
| (Light chain) | cgccaccggc gtgcactccg acatccagat gacccagtcc ccctcctccc |
| (10) | tgtccgcctc cgtgggcgac |
| | cgggtgacca tcacctgctc cgcctcccag gacatctcca actacctgaa |
| | ctggtaccag cagaagcccg gcaaggcccc caaggtgctg atctacttca |
| | cctcctccct gcactccggc |
| | gtgccctccc ggttctccgg ctccggctcc ggcaccgact tcaccctgac |
| | catctcctcc ctgcagcccg aggacttcgc cacctactac tgccagcagt |
| | actccaccgt gccctggacc |
| | ttcggccagg gcaccaaggt ggagatcaag cggaccgtgg ccgccccctc |
| | cgtgttcatc ttccccccct ccgacgagca gctgaagtcc ggcaccgcct |
| | ccgtggtgtg cctgctgaac |
| | aacttctacc cccgggaggc caaggtgcag tggaaggtgg acaacgccct |
| | gcagtccggc aactcccagg agtccgtgac cgagcaggac tccaaggact |
| | ccacctactc cctgtcctcc |
| | accctgaccc tgtccaaggc cgactacgag aagcacaagg tgtacgcctg |
| | cgaggtgacc caccagggcc tgtcctcccc cgtgaccaag tccttcaacc |
| | ggggcgagtg ctgagcggcc gcctcgag |
| Bevacizumab cDNA | gctagcgcca ccatgggctg gtcctgcatc atcctgttcc tggtggccac |
| (Heavy chain) | cgccaccggc gtgcactccg aggtgcagct ggtggagtcc ggcggcggcc |
| (11) | tggtgcagcc cggcggctcc |
| | ctgcggctgt cctgcgccgc ctccggctac accttcacca actacggcat |
| | gaactgggtg cggcaggccc ccggcaaggg cctggagtgg gtgggctgga |
| | tcaacaccta caccggcgag |
| | cccacctacg ccgccgactt caagcggcgg ttcaccttct ccctggacac |
| | ctccaagtcc accgcctacc tgcagatgaa ctccctgcgg gccgaggaca |
| | ccgccgtgta ctactgcgcc |
| | aagtaccccc actactacgg ctcctcccac tggtacttcg acgtgtgggg |
| | ccagggcacc ctggtgaccg tgtcctccgc ctccaccaag ggcccctccg |
| | tgttccccct ggccccctcc |
| | tccaagtcca cctccggcgg caccgccgcc ctgggctgcc tggtgaagga |
| | ctacttcccc gagcccgtga ccgtgtcctg gaactccggc gccctgacct |
| | ccggcgtgca caccttcccc |
| | gccgtgctgc agtcctccgg cctgtactcc ctgtcctccg tggtgaccgt |
| | gccctcctcc tccctgggca cccagaccta catctgcaac gtgaaccaca |
| | agccctccaa caccaaggtg |
| | gacaagaagg tggagcccaa gtcctgcgac aagacccaca cctgcccccc |
| | ctgccccgcc cccgagctgc tgggcggccc ctccgtgttc ctgttccccc |
| | ccaagcccaa ggacaccctg |
| | atgatctccc ggacccccga ggtgacctgc gtggtggtgg acgtgtccca |
| | cgaggacccc gaggtgaagt tcaactggta cgtggacggc gtggaggtgc |
| | acaacgccaa gaccaagccc |
| | cgggaggagc agtacaactc cacctaccgg gtggtgtccg tgctgaccgt |
| | gctgcaccag gactggctga acggcaagga gtacaagtgc aaggtgtcca |
| | acaaggccct gcccgccccc |
| | atcgagaaga ccatctccaa ggccaagggc cagccccggg agccccaggt |
| | gtacaccctg cccccctccc gggaggagat gaccaagaac caggtgtccc |
| | tgacctgcct ggtgaagggc |
| | ttctacccct ccgacatcgc cgtggagtgg gagtccaacg gccagcccga |
| | gaacaactac aagaccaccc cccccgtgct ggactccgac ggctccttct |
| | tcctgtactc caagctgacc |
| | gtggacaagt cccggtggca gcagggcaac gtgttctcct gctccgtgat |
| | gcacgaggcc ctgcacaacc actacaccca gaagtccctg tccctgtccc |
| | ccggcaagtg agcggccgcc |
| Bevacizumab Fab | DIQMTQSPSSLSASVGDRVTITCSASQDISNYLNWYQQKPGKAPKVLIYFTSSLH |
| Amino Acid | SGVPSRFSGSGSGTDFTLTISSLQPEDFATYYCQQYSTVPWTFGQGTKVEIKRTV |
| Sequence (Light | AAPSVFIFPPSDEQLKSGTASVVCLLNNFYPREAKVQWKVDNALQSGNSQESVTE |
| chain) | QDSKDSTYSLSSTLTLSKADYEKHKVYACEVTHQGLSSPVTKSFNRGEC |
| (3) | |
| Bevacizumab Fab | EVQLVESGGGLVQPGGSLRLSCAASGYTFTNYGMNWVRQAPGKGLEWVGWINTYT |
| Amino Acid | GEPTYAADFKRRFTFSLDTSKSTAYLQMNSLRAEDTAVYYCAKYPHYYGSSHWYF |
| Sequence (Heavy | DVWGQGTLVTVSSASTKGPSVFPLAPSSKSTSGGTAALGCLVKDYFPEPVTVSWN |
| chain) | SGALTSGVHTFPAVLQSSGLYSLSSVVTVPSSSLGTQTYICNVNHKPSNTKVDKK |
| (4) | VEPKSCDKTHL |
| Ranibizumab cDNA | gagctccatg gagtttttca aaaagacggc acttgccgca ctggttatgg |
| (Light chain | gttttagtgg tgcagcattg gccgatatcc agctgaccca gagcccgagc |
| comprising a | agcctgagcg caagcgttgg tgatcgtgtt accattacct gtagcgcaag |
| signal sequence) | ccaggatatt agcaattatc tgaattggta tcagcagaaa ccgggtaaag |
| (12) | caccgaaagt tctgatttat tttaccagca gcctgcatag cggtgttccg |
| | agccgtttta gcggtagcgg tagtggcacc gattttaccc tgaccattag |
| | cagcctgcag ccggaagatt ttgcaaccta ttattgtcag cagtatagca |
| | ccgttccgtg gacctttggt cagggcacca aagttgaaat taaacgtacc |
| | gttgcagcac cgagcgtttt tatttttccg cctagtgatg aacagctgaa |
| | aagcggcacc gcaagcgttg tttgtctgct gaataatttt tatccgcgtg |
| | aagcaaaagt gcagtggaaa gttgataatg cactgcagag cggtaatagc |
| | caagaaagcg ttaccgaaca ggatagcaaa gatagcacct atagcctgag |
| | cagcaccctg accctgagca aagcagatta tgaaaaacac aaagtgtatg |
| | cctgcgaagt tacccatcag ggtctgagca gtccggttac caaaagtttt |
| | aatcgtggcg aatgctaata gaagcttggt acc |
| Ranibizumab cDNA | gagctcatat gaaatacctg ctgccgaccg ctgctgctgg tctgctgctc |
| (Heavy chain | ctcgctgccc agccggcgat ggccgaagtt cagctggttg aaagcggtgg |
| comprising a | tggtctggtt cagcctggtg gtagcctgcg tctgagctgt gcagcaagcg |
| signal sequence) | gttatgattt tacccattat ggtatgaatt gggttcgtca ggcaccgggt |
| (13) | aaaggtctgg aatgggttgg ttggattaat acctataccg gtgaaccgac |
| | ctatgcagca gattttaaac gtcgttttac ctttagcctg gataccagca |
| | aaagcaccgc atatctgcag atgaatagcc tgcgtgcaga agataccgca |
| | gtttattatt gtgccaaata tccgtattac tatggcacca gccactggta |
| | tttcgatgtt tggggtcagg gcaccctggt taccgttagc agcgcaagca |
| | ccaaaggtcc gagcgttttt ccgctggcac cgagcagcaa aagtaccagc |
| | ggtggcacag cagcactggg ttgtctggtt aaagattatt ttccggaacc |
| | ggttaccgtg agctggaata gcggtgcact gaccagcggt gttcatacct |
| | ttccggcagt tctgcagagc agcggtctgt atagcctgag cagcgttgtt |
| | accgttccga gcagcagcct gggcacccag acctatattt gtaatgttaa |
| | tcataaaccg agcaatacca aagtggataa aaaagttgag ccgaaaagct |
| | gcgataaaac ccatctgtaa tagggtacc |
| Ranibizumab Fab | DIQLTQSPSSLSASVGDRVTITCSASQDISNYLNWYQQKPGKAPKVLIYFTSSLH |
| Amino Acid | SGVPSRFSGSGSGTDFTLTISSLQPEDFATYYCQQYSTVPWTFGQGTKVEIKRTV |
| Sequence (Light | AAPSVFIFPPSDEQLKSGTASVVCLLNNFYPREAKVQWKVDNALQSGNSQESVTE |
| chain) | QDSKDSTYSLSSTLTLSKADYEKHKVYACEVTHQGLSSPVTKSFNRGEC |
| (1) | |
| Ranibizumab Fab | EVQLVESGGGLVQPGGSLRLSCAASGYDFTHYGMNWVRQAPGKGLEWVGWINTYT |
| Amino Acid | GEPTYAADFKRRFTFSLDTSKSTAYLQMNSLRAEDTAVYYCAKYPYYYGTSHWYF |
| Sequence (Heavy | DVWGQGTLVTVSSASTKGPSVFPLAPSSKSTSGGTAALGCLVKDYFPEPVTVSWN |
| chain) | SGALTSGVHTFPAVLQSSGLYSLSSVVTVPSSSLGTQTYICNVNHKPSNTKVDKK |
| (2) | VEPKSCDKTHL |
| Bevacizumab Light | SASQDISNYLN |
| Chain CDRs | FTSSLHS |
| (14, 15, and 16) | QQYSTVPWT |
| Bevacizumab Heavy | GYTFTNYGMN |
| Chain CDRs | WINTYTGEPTYAADFKR |
| (17, 18, and 19) | YPHYYGSSHWYFDV |
| Ranibizumab Light | SASQDISNYLN |
| Chain CDRs | FTSSLHS |
| (14, 15, and 16) | QQYSTVPWT |
| Ranibizumab Heavy | GYDFTHYGMN |
| Chain CDRs | WINTYTGEPTYAADFKR |
| (20, 18, and 21) | YPYYYGTSHWYFDV |

5.2.9 Constructs

In certain embodiments, the viral vectors provided herein comprise the following elements in the following order: a) a constitutive or a hypoxia-inducible promoter sequence, and b) a sequence encoding the transgene (e.g., an anti-VEGF antigen-binding fragment moiety). In certain embodiments, the sequence encoding the transgene comprises multiple ORFs separated by IRES elements. In certain embodiments, the ORFs encode the heavy and light chain domains of the anti-VEGF antigen-binding fragment. In certain embodiments, the sequence encoding the transgene comprises multiple subunits in one ORF separated by F/F2A sequences. In certain embodiments, the sequence comprising the transgene encodes the heavy and light chain domains of the anti-VEGF antigen-binding fragment separated by an F/F2A sequence. In certain embodiments, the viral vectors provided herein comprise the following elements in the following order: a) a constitutive or a hypoxia-inducible promoter sequence, and b) a sequence encoding the transgene (e.g., an anti-VEGF antigen-binding fragment moiety), wherein the transgene comprises the signal peptide of VEGF (SEQ ID NO: 5), and wherein the transgene encodes a light chain and a heavy chain sequence separated by an IRES element. In certain embodiments, the viral vectors provided herein comprise the following elements in the following order: a) a constitutive or a hypoxia-inducible promoter sequence, and b) a sequence encoding the transgene (e.g., an anti-VEGF antigen-binding fragment moiety), wherein the transgene comprises the signal peptide of VEGF (SEQ ID NO: 5), and wherein the transgene encodes a light chain and a heavy chain sequence separated by a cleavable F/F2A sequence.

In certain embodiments, the viral vectors provided herein comprise the following elements in the following order: a) a first ITR sequence, b) a first linker sequence, c) a constitutive or a hypoxia-inducible promoter sequence, d) a second linker sequence, e) an intron sequence, f) a third linker sequence, g) a first UTR sequence, h) a sequence encoding the transgene (e.g., an anti-VEGF antigen-binding fragment moiety), i) a second UTR sequence, j) a fourth linker sequence, k) a poly A sequence, l) a fifth linker sequence, and m) a second ITR sequence.

In certain embodiments, the viral vectors provided herein comprise the following elements in the following order: a) a first ITR sequence, b) a first linker sequence, c) a constitutive or a hypoxia-inducible promoter sequence, d) a second linker sequence, e) an intron sequence, f) a third linker sequence, g) a first UTR sequence, h) a sequence encoding the transgene (e.g., an anti-VEGF antigen-binding fragment moiety), i) a second UTR sequence, j) a fourth linker sequence, k) a poly A sequence, l) a fifth linker sequence, and m) a second ITR sequence, wherein the transgene comprises the signal peptide of VEGF (SEQ ID NO: 5), and wherein the transgene encodes a light chain and a heavy chain sequence separated by a cleavable F/F2A sequence.

5.2.10 Manufacture and Testing of Vectors

The viral vectors provided herein may be manufactured using host cells. The viral vectors provided herein may be manufactured using mammalian host cells, for example, A549, WEHI, 10T1/2, BHK, MDCK, COS1, COS7, BSC 1, BSC 40, BMT 10, VERO, W138, HeLa, 293, Saos, C2C12, L, HT1080, HepG2, primary fibroblast, hepatocyte, and myoblast cells. The viral vectors provided herein may be manufactured using host cells from human, monkey, mouse, rat, rabbit, or hamster.

The host cells are stably transformed with the sequences encoding the transgene and associated elements (i.e., the vector genome), and the means of producing viruses in the host cells, for example, the replication and capsid genes (e.g., the rep and cap genes of AAV). For a method of producing recombinant AAV vectors with AAV8 capsids, see Section IV of the Detailed Description of U.S. Pat. No. 7,282,199 B2, which is incorporated herein by reference in its entirety. Genome copy titers of said vectors may be determined, for example, by TAQMAN® analysis. Virions may be recovered, for example, by CsCl₂ sedimentation.

In vitro assays, e.g., cell culture assays, can be used to measure transgene expression from a vector described herein, thus indicating, e.g., potency of the vector. For example, the PER.C6® Cell Line (Lonza), a cell line derived from human embryonic retinal cells, or retinal pigment epithelial cells, e.g., the retinal pigment epithelial cell line hTERT RPE-1 (available from ATCC®), can be used to assess transgene expression. Once expressed, characteristics of the expressed product (i.e., HuGlyFabVEGFi) can be determined, including determination of the glycosylation and tyrosine sulfation patterns associated with the HuGlyFabVEGFi. Glycosylation patterns and methods of determining the same are discussed in Section 5.1.1, while tyrosine sulfation patterns and methods of determining the same are discussed in Section 5.1.2. In addition, benefits resulting from glycosylation/sulfation of the cell-expressed HuGlyFabVEGFi can be determined using assays known in the art, e.g., the methods described in Sections 5.1.1 and 5.1.2.

5.2.11 Compositions

Compositions are described comprising a vector encoding a transgene described herein and a suitable carrier. A suitable carrier (e.g., for subretinal and/or intraretinal administration) would be readily selected by one of skill in the art.

### 5.3 Gene Therapy

Methods are described for the administration of a therapeutically effective amount of a transgene construct to human subjects having an ocular disease caused by increased neovascularization. More particularly, methods for administration of a therapeutically effective amount of a transgene construct to patients having AMD, in particular, for subretinal and/or intraretinal administration are described. In particular embodiments, such methods for subretinal and/or intraretinal administration of a therapeutically effective amount of a transgene construct can be used to treat to patients having wet AMD or diabetic retinopathy.

Methods are described for subretinal and/or intraretinal administration of a therapeutically effective amount of a transgene construct to patients diagnosed with an ocular disease caused by increased neovascularization. In particular embodiments, such methods for subretinal and/or intraretinal administration of a therapeutically effective amount of a transgene construct to can be used to treat patients diagnosed with AMD; and in particular, wet AMD (neovascular AMD), or diabetic retinopathy.

Also provided herein are methods for subretinal and/or intraretinal administration of a therapeutically effective amount of a transgene construct and methods of administration of a therapeutically effective amount of a transgene construct to the retinal pigment epithelium.

5.3.1 Target Patient Populations

In certain embodiments, the methods provided herein are for the administration to patients diagnosed with an ocular disease caused by increased neovascularization.

In certain embodiments, the methods provided herein are for the administration to patients diagnosed with severe AMD. In certain embodiments, the methods provided herein are for the administration to patients diagnosed with attenuated AMD.

In certain embodiments, the methods provided herein are for the administration to patients diagnosed with severe wet AMD. In certain embodiments, the methods provided herein are for the administration to patients diagnosed with attenuated wet AMD.

In certain embodiments, the methods provided herein are for the administration to patients diagnosed with severe diabetic retinopathy. In certain embodiments, the methods provided herein are for the administration to patients diagnosed with attenuated diabetic retinopathy.

In certain embodiments, the methods provided herein are for the administration to patients diagnosed with AMD who have been identified as responsive to treatment with an anti-VEGF antibody.

In certain embodiments, the methods provided herein are for the administration to patients diagnosed with AMD who have been identified as responsive to treatment with an anti-VEGF antigen-binding fragment.

In certain embodiments, the methods provided herein are for the administration to patients diagnosed with AMD who have been identified as responsive to treatment with an anti-VEGF antigen-binding fragment injected intravitreally prior to treatment with gene therapy.

In certain embodiments, the methods provided herein are for the administration to patients diagnosed with AMD who have been identified as responsive to treatment with LUCENTIS® (ranibizumab), EYLEA® (aflibercept), and/or AVASTIN® (bevacizumab).

5.3.2 Dosage and Mode of Administration

Therapeutically effective doses of the recombinant vector should be delivered subretinally in an injection volume ranging from ≥0.1 mL to ≤0.5 mL, preferably in 0.1 to 0.30 mL (100-300 μl), and most preferably, in a volume of 0.25 mL (250 μl). Doses that maintain a concentration of the transgene product at a Cₘᵢₙ of at least 0.330 μg/mL in the Vitreous humour, or 0.110 μg/mL in the Aqueous humour (the anterior chamber of the eye) for three months are desired; thereafter, Vitreous Cₘᵢₙ concentrations of the transgene product ranging from 1.70 to 6.60 μg/mL, and/or Aqueous Cₘᵢₙ concentrations ranging from 0.567 to 2.20 μg/mL should be maintained. However, because the transgene product is continuously produced (under the control of a constitutive promoter or induced by hypoxic conditions when using an hypoxia-inducible promoter), maintenance of lower concentrations can be effective. Vitreous humour concentrations can be measured directly in patient samples of fluid collected from the vitreous humour or the anterior chamber, or estimated and/or monitored by measuring the patient's serum concentrations of the transgene product—the ratio of systemic to vitreal exposure to the transgene product is about 1:90,000. (E.g., see, vitreous humor and serum concentrations of ranibizumab reported in Xu L, et al., 2013, Invest. Opthal. Vis. Sci. 54: 1616-1624, at p. 1621 and Table 5 at p. 1623, which is incorporated by reference herein in its entirety).

In certain embodiments, dosages are measured by the number of genome copies administered to the eye of the patient (e.g., injected subretinally and/or intraretinally). In certain embodiments, 1×10⁹ to 1×10¹¹ genome copies are administered. In specific embodiments, 1×10⁹ to 5×10⁹ genome copies are administered. In specific embodiments, 6×10⁹ to 3×10¹⁰ genome copies are administered. In specific embodiments, 4×10¹⁰ to 1×10¹¹ genome copies are administered.

5.3.3 Sampling and Monitoring of Efficacy

Effects of the methods of treatment provided herein on visual deficits may be measured by BCVA (Best-Corrected Visual Acuity), intraocular pressure, slit lamp biomicroscopy, and/or indirect ophthalmoscopy.

Effects of the methods of treatment provided herein on physical changes to eye/retina may be measured by SD-OCT (SD-Optical Coherence Tomography).

Efficacy may be monitored as measured by electroretinography (ERG).

Effects of the methods of treatment provided herein may be monitored by measuring signs of vision loss, infection, inflammation and other safety events, including retinal detachment.

Retinal thickness may be monitored to determine efficacy of the treatments provided herein. Without being bound by any particular theory, thickness of the retina may be used as a clinical readout, wherein the greater reduction in retinal thickness or the longer period of time before thickening of the retina, the more efficacious the treatment. Retinal function may be determined, for example, by ERG. ERG is a non-invasive electrophysiologic test of retinal function, approved by the FDA for use in humans, which examines the light sensitive cells of the eye (the rods and cones), and their connecting ganglion cells, in particular, their response to a flash stimulation. Retinal thickness may be determined, for example, by SD-OCT. SD-OCT is a three-dimensional imaging technology which uses low-coherence interferometry to determine the echo time delay and magnitude of backscattered light reflected off an object of interest. OCT can be used to scan the layers of a tissue sample (e.g., the retina) with 3 to 15 μm axial resolution, and SD-OCT improves axial resolution and scan speed over previous forms of the technology (Schuman, 2008, Trans. Am. Opthamol. Soc. 106:426-458).

### 5.4 Combination Therapies

The methods of treatment provided herein may be combined with one or more additional therapies. In one aspect, the methods of treatment provided herein are administered with laser photocoagulation. In one aspect, the methods of treatment provided herein are administered with photodynamic therapy with verteporfin.

In one aspect, the methods of treatment provided herein are administered with intravitreal (IVT) injections with anti-VEGF agents, including but not limited to HuPTMFabVEGFi, e.g., HuGlyFabVEGFi produced in human cell lines (Dumont et al., 2015, supra), or other anti-VEGF agents such as pegaptanib, ranibizumab, aflibercept, or bevacizumab.

The additional therapies may be administered before, concurrently or subsequent to the gene therapy treatment.

The efficacy of the gene therapy treatment may be indicated by the elimination of or reduction in the number of rescue treatments using standard of care, for example, intravitreal injections with anti-VEGF agents, including but not limited to HuPTMFabVEGFi, e.g., HuGlyFabVEGFi produced in human cell lines, or other anti-VEGF agents such as pegaptanib, ranibizumab, aflibercept, or bevacizumab.

TABLE 3
TABLE OF SEQUENCES

| SEQ | | |
|---|---|---|
| ID | | |
| NO: | Description | Sequence |
| 1 | Ranibizumab Fab | DIQLTQSPSSLSASVGDRVTITCSASQDISNYLNWYQQKPGKAPKVLIYFTSSLH |
| | Amino Acid | SGVPSRFSGSGSGTDFTLTISSLQPEDFATYYCQQYSTVPWTFGQGTKVEIKRTV |
| | Sequence (Light | AAPSVFIFPPSDEQLKSGTASVVCLLNNFYPREAKVQWKVDNALQSGNSQESVTE |
| | chain) | QDSKDSTYSLSSTLTLSKADYEKHKVYACEVTHQGLSSPVTKSFNRGEC |
| 2 | Ranibizumab Fab | EVQLVESGGGLVQPGGSLRLSCAASGYDFTHYGMNWVRQAPGKGLEWVGWINTYT |
| | Amino Acid | GEPTYAADFKRRFTFSLDTSKSTAYLQMNSLRAEDTAVYYCAKYPYYYGTSHWYF |
| | Sequence (Heavy | DVWGQGTLVTVSSASTKGPSVFPLAPSSKSTSGGTAALGCLVKDYFPEPVTVSWN |
| | chain) | SGALTSGVHTFPAVLQSSGLYSLSSVVTVPSSSLGTQTYICNVNHKPSNTKVDKK |
| | | VEPKSCDKTHL |
| 3 | Bevacizumab Fab | DIQMTQSPSSLSASVGDRVTITCSASQDISNYLNWYQQKPGKAPKVLIYFTSSLH |
| | Amino Acid | SGVPSRFSGSGSGTDFTLTISSLQPEDFATYYCQQYSTVPWTFGQGTKVEIKRTV |
| | Sequence (Light | AAPSVFIFPPSDEQLKSGTASVVCLLNNFYPREAKVQWKVDNALQSGNSQESVTE |
| | chain) | QDSKDSTYSLSSTLTLSKADYEKHKVYACEVTHQGLSSPVTKSFNRGEC |
| 4 | Bevacizumab Fab | EVQLVESGGGLVQPGGSLRLSCAASGYTFTNYGMNWVRQAPGKGLEWVGWINTYT |
| | Amino Acid | GEPTYAADFKRRFTFSLDTSKSTAYLQMNSLRAEDTAVYYCAKYPHYYGSSHWYF |
| | Sequence (Heavy | DVWGQGTLVTVSSASTKGPSVFPLAPSSKSTSGGTAALGCLVKDYFPEPVTVSWN |
| | chain) | SGALTSGVHTFPAVLQSSGLYSLSSVVTVPSSSLGTQTYICNVNHKPSNTKVDKK |
| | | VEPKSCDKTHL |
| 5 | VEGF-A signal | MNFLLSWVHW SLALLLYLHH AKWSQA |
| | peptide | |
| 6 | Fibulin-1 signal | MERAAPSRRV PLPLLLLGGL ALLAAGVDA |
| | peptide | |
| 7 | Vitronectin | MAPLRPLLIL ALLAWVALA |
| | signal peptide | |
| 8 | Complement | MRLLAKIICLMLWAICVA |
| | Factor H signal | |
| | peptide | |
| 9 | Opticin signal | MRLLAFLSLL ALVLQETGT |
| | peptide | |
| 10 | Bevacizumab | gctagcgcca ccatgggctg gtcctgcatc atcctgttcc tggtggccac |
| | cDNA | cgccaccggc gtgcactccg acatccagat gacccagtcc ccctcctccc |
| | (Light chain) | tgtccgcctc cgtgggcgac |
| | | cgggtgacca tcacctgctc cgcctcccag gacatctcca actacctgaa |
| | | ctggtaccag cagaagcccg gcaaggcccc caaggtgctg atctacttca |
| | | cctcctccct gcactccggc |
| | | gtgccctccc ggttctccgg ctccggctcc ggcaccgact tcaccctgac |
| | | catctcctcc ctgcagcccg aggacttcgc cacctactac tgccagcagt |
| | | actccaccgt gccctggacc |
| | | ttcggccagg gcaccaaggt ggagatcaag cggaccgtgg ccgccccctc |
| | | cgtgttcatc ttccccccct ccgacgagca gctgaagtcc ggcaccgcct |
| | | ccgtggtgtg cctgctgaac |
| | | aacttctacc cccgggaggc caaggtgcag tggaaggtgg acaacgccct |
| | | gcagtccggc aactcccagg agtccgtgac cgagcaggac tccaaggact |
| | | ccacctactc cctgtcctcc |
| | | accctgaccc tgtccaaggc cgactacgag aagcacaagg tgtacgcctg |
| | | cgaggtgacc caccagggcc tgtcctcccc cgtgaccaag tccttcaacc |
| | | ggggcgagtg ctgagcggcc gcctcgag |
| 11 | Bevacizumab | gctagcgcca ccatgggctg gtcctgcatc atcctgttcc tggtggccac |
| | cDNA (Heavy | cgccaccggc gtgcactccg aggtgcagct ggtggagtcc ggcggcggcc |
| | chain) | tggtgcagcc cggcggctcc |
| | | ctgcggctgt cctgcgccgc ctccggctac accttcacca actacggcat |
| | | gaactgggtg cggcaggccc ccggcaaggg cctggagtgg gtgggctgga |
| | | tcaacaccta caccggcgag |
| | | cccacctacg ccgccgactt caagcggcgg ttcaccttct ccctggacac |
| | | ctccaagtcc accgcctacc tgcagatgaa ctccctgcgg gccgaggaca |
| | | ccgccgtgta ctactgcgcc |
| | | aagtaccccc actactacgg ctcctcccac tggtacttcg acgtgtgggg |
| | | ccagggcacc ctggtgaccg tgtcctccgc ctccaccaag ggcccctccg |
| | | tgttccccct ggccccctcc |
| | | tccaagtcca cctccggcgg caccgccgcc ctgggctgcc tggtgaagga |
| | | ctacttcccc gagcccgtga ccgtgtcctg gaactccggc gccctgacct |
| | | ccggcgtgca caccttcccc |
| | | gccgtgctgc agtcctccgg cctgtactcc ctgtcctccg tggtgaccgt |
| | | gccctcctcc tccctgggca cccagaccta catctgcaac gtgaaccaca |
| | | agccctccaa caccaaggtg |
| | | gacaagaagg tggagcccaa gtcctgcgac aagacccaca cctgcccccc |
| | | ctgccccgcc cccgagctgc tgggcggccc ctccgtgttc ctgttccccc |
| | | ccaagcccaa ggacaccctg |
| | | atgatctccc ggacccccga ggtgacctgc gtggtggtgg acgtgtccca |
| | | cgaggacccc gaggtgaagt tcaactggta cgtggacggc gtggaggtgc |
| | | acaacgccaa gaccaagccc |
| | | cgggaggagc agtacaactc cacctaccgg gtggtgtccg tgctgaccgt |
| | | gctgcaccag gactggctga acggcaagga gtacaagtgc aaggtgtcca |
| | | acaaggccct gcccgccccc |
| | | atcgagaaga ccatctccaa ggccaagggc cagccccggg agccccaggt |
| | | gtacaccctg cccccctccc gggaggagat gaccaagaac caggtgtccc |
| | | tgacctgcct ggtgaagggc |
| | | ttctacccct ccgacatcgc cgtggagtgg gagtccaacg gccagcccga |
| | | gaacaactac aagaccaccc cccccgtgct ggactccgac ggctccttct |
| | | tcctgtactc caagctgacc |
| | | gtggacaagt cccggtggca gcagggcaac gtgttctcct gctccgtgat |
| | | gcacgaggcc ctgcacaacc actacaccca gaagtccctg tccctgtccc |
| | | ccggcaagtg agcggccgcc |
| 12 | Ranibizumab | gagctccatg gagtttttca aaaagacggc acttgccgca ctggttatgg |
| | cDNA (Light | gttttagtgg tgcagcattg gccgatatcc agctgaccca gagcccgagc |
| | chain comprising | agcctgagcg caagcgttgg tgatcgtgtt accattacct gtagcgcaag |
| | a signal | ccaggatatt agcaattatc tgaattggta tcagcagaaa ccgggtaaag |
| | sequence) | caccgaaagt tctgatttat tttaccagca gcctgcatag cggtgttccg |
| | | agccgtttta gcggtagcgg tagtggcacc gattttaccc tgaccattag |
| | | cagcctgcag ccggaagatt ttgcaaccta ttattgtcag cagtatagca |
| | | ccgttccgtg gacctttggt cagggcacca aagttgaaat taaacgtacc |
| | | gttgcagcac cgagcgtttt tatttttccg cctagtgatg aacagctgaa |
| | | aagcggcacc gcaagcgttg tttgtctgct gaataatttt tatccgcgtg |
| | | aagcaaaagt gcagtggaaa gttgataatg cactgcagag cggtaatagc |
| | | caagaaagcg ttaccgaaca ggatagcaaa gatagcacct atagcctgag |
| | | cagcaccctg accctgagca aagcagatta tgaaaaacac aaagtgtatg |
| | | cctgcgaagt tacccatcag ggtctgagca gtccggttac caaaagtttt |
| | | aatcgtggcg aatgctaata gaagcttggt acc |
| 13 | Ranibizumab | gagctcatat gaaatacctg ctgccgaccg ctgctgctgg tctgctgctc |
| | cDNA (Heavy | ctcgctgccc agccggcgat ggccgaagtt cagctggttg aaagcggtgg |
| | chain comprising | tggtctggtt cagcctggtg gtagcctgcg tctgagctgt gcagcaagcg |
| | a signal | gttatgattt tacccattat ggtatgaatt gggttcgtca ggcaccgggt |
| | sequence) | aaaggtctgg aatgggttgg ttggattaat acctataccg gtgaaccgac |
| | | ctatgcagca gattttaaac gtcgttttac ctttagcctg gataccagca |
| | | aaagcaccgc atatctgcag atgaatagcc tgcgtgcaga agataccgca |
| | | gtttattatt gtgccaaata tccgtattac tatggcacca gccactggta |
| | | tttcgatgtt tggggtcagg gcaccctggt taccgttagc agcgcaagca |
| | | ccaaaggtcc gagcgttttt ccgctggcac cgagcagcaa aagtaccagc |
| | | ggtggcacag cagcactggg ttgtctggtt aaagattatt ttccggaacc |
| | | ggttaccgtg agctggaata gcggtgcact gaccagcggt gttcatacct |
| | | ttccggcagt tctgcagagc agcggtctgt atagcctgag cagcgttgtt |
| | | accgttccga gcagcagcct gggcacccag acctatattt gtaatgttaa |
| | | tcataaaccg agcaatacca aagtggataa aaaagttgag ccgaaaagct |
| | | gcgataaaac ccatctgtaa tagggtacc |
| 14 | Bevacizumab and | SASQDISNYLN |
| | Ranibizumab | |
| | Light Chain | |
| | CDR1 | |
| 15 | Bevacizumab and | FTSSLHS |
| | Ranibizumab | |
| | Light Chain | |
| | CDR2 | |
| 16 | Bevacizumab and | QQYSTVPWT |
| | Ranibizumab | |
| | Light Chain | |
| | CDR3 | |
| 17 | Bevacizumab | GYTFTNYGMN |
| | Heavy Chain | |
| | CDR1 | |
| 18 | Bevacizumab and | WINTYTGEPTYAADFKR |
| | Ranibizumab | |
| | Heavy Chain | |
| | CDR2 | |
| 19 | Bevacizumab | YPHYYGSSHWYFDV |
| | Heavy Chain | |
| | CDR3 | |
| 20 | Ranibizumab | GYDFTHYGMN |
| | Heavy Chain | |
| | CDR1 | |
| 21 | Ranibizumab | YPYYYGTSHWYFDV |
| | Heavy Chain | |
| | CDR3 | |
| 22 | Albumin signal | MKWVTFISLLFLFSSAYS |
| | peptide | |
| 23 | Chymotrypsinoge | MAFLWLLSCWALLGTTFG |
| | n signal peptide | |
| 24 | Interleukin-2 | MYRMQLLSCIALILALVTNS |
| | signal peptide | |
| 25 | Trypsinogen-2 | MNLLLILTFVAAAVA |
| | signal peptide | |
| 26 | F2A site | LLNFDLLKLAGDVESNPGP |
| 27 | T2A site | (GSG)EGRGSLLTCGDVEENPGP |
| 28 | P2A site | (GSG)ATNFSLLKQAGDVEENPGP |
| 29 | E2A site | (GSG)QCTNYALLKLAGDVESNPGP |
| 30 | F2A site | (GSG)VKQTLNFDLLKLAGDVESNPGP |
| 31 | Furin linker | RKRR |
| 32 | Furin linker | RRRR |
| 33 | Furin linker | RRKR |
| 34 | Furin linker | RKKR |
| 35 | Furin linker | R-X-K/R-R |
| 36 | Furin linker | RXKR |
| 37 | Furin linker | RXRR |

### 6. EXAMPLES

### 6.1 Example 1: Bevacizumab Fab cDNA-Based Vector

A Bevacizumab Fab cDNA-based vector is constructed comprising a transgene comprising Bevacizumab Fab portion of the light and heavy chain cDNA sequences (SEQ ID NOs. 10 and 11, respectively). The transgene also comprises nucleic acids comprising a signal peptide chosen from the group listed in Table 1. The nucleotide sequences encoding the light chain and heavy chain are separated by IRES elements or 2A cleavage sites to create a bicistronic vector. Optionally, the vector additionally comprises a hypoxia-inducible promoter.

### 6.2 Example 2: Ranibizumab cDNA-Based Vector

A Ranibizumab Fab cDNA-based vector is constructed comprising a transgene comprising Ranibizumab Fab light and heavy chain cDNAs (the portions of SEQ ID NOs.12 and 13, respectively not encoding the signal peptide). The transgene also comprises nucleic acids comprising a signal peptide chosen from the group listed in Table 1. The nucleotide sequences encoding the light chain and heavy chain are separated by IRES elements or 2A cleavage sites to create a bicistronic vector. Optionally, the vector additionally comprises a hypoxia-inducible promoter.

### 6.3 Example 3: Hyperglycosylated Bevacizumab Fab cDNA-Based Vector

A hyperglycosylated Bevacizumab Fab cDNA-based vector is constructed comprising a transgene comprising Bevacizumab Fab portion of the light and heavy chain cDNA sequences (SEQ ID NOs. 10 and 11, respectively) with mutations to the sequence encoding one or more of the following mutations: L118N (heavy chain), E195N (light chain), or Q160N or Q160S (light chain). The transgene also comprises nucleic acids comprising a signal peptide chosen from the group listed in Table 1. The nucleotide sequences encoding the light chain and heavy chain are separated by IRES elements or 2A cleavage sites to create a bicistronic vector. Optionally, the vector additionally comprises a hypoxia-inducible promoter.

### 6.4 Example 4: Hyperglycosylated Ranibizumab cDNA-based Vector

A hyperglycosylated Ranibizumab Fab cDNA-based vector is constructed comprising a transgene comprising Ranibizumab Fab light and heavy chain cDNAs (the portions of SEQ ID NOs.12 and 13, respectively not encoding the signal peptide), with mutations to the sequence encoding one or more of the following mutations: L118N (heavy chain), E195N (light chain), or Q160N or Q160S (light chain). The transgene also comprises nucleic acids comprising a signal peptide chosen from the group listed in Table 1. The nucleotide sequences encoding the light chain and heavy chain are separated by IRES elements or 2A cleavage sites to create a bicistronic vector. Optionally, the vector additionally comprises a hypoxia-inducible promoter.

### 6.5 Example 5: Ranibizumab Based HuGlyFabVEGFi

A ranibizumab Fab cDNA-based vector (see Example 2) is expressed in the PER.C6® Cell Line (Lonza) in the AAV8 background. The resultant product, ranibizumab-based HuGlyFabVEGFi is determined to be stably produced. N-glycosylation of the HuGlyFabVEGFi is confirmed by hydrazinolysis and MS/MS analysis. See, e.g., Bondt et al., Mol. & Cell. Proteomics 13.11:3029-3039. Based on glycan analysis, HuGlyFabVEGFi is confirmed to be N-glycosylated, with 2,6 sialic acid a predominant modification. Advantageous properties of the N-glycosylated HuGlyFabVEGFi are determined using methods known in the art. The HuGlyFabVEGFi can be found to have increased stability and increased affinity for its antigen (VEGF). See Sola and Griebenow, 2009, J Pharm Sci., 98(4): 1223-1245 for methods of assessing stability and Wright et al., 1991, EMBO J. 10:2717-2723 and Leibiger et al., 1999, Biochem. J. 338:529-538 for methods of assessing affinity.

### 6.6 Example 6: Treatment of Wet AMD with Ranibizumab Based HuGlyFabVEGFi

Based on determination of advantageous characteristics of ranibizumab-based HuGlyFabVEGFi (see Example 5), a ranibizumab Fab cDNA-based vector is deemed useful for treatment of wet AMD when expressed as a transgene. A subject presenting with wet AMD is administered AAV8 that encodes ranibizumab Fab at a dose sufficient to that a concentration of the transgene product at a Cmin of at least 0.330 μg/mL in the Vitreous humour for three months. Following treatment, the subject is evaluated for improvement in symptoms of wet AMD.

### 6.7 Example 7: A Single Dose Subretinal Administration Reduces Retinal Neovascularization in Transgenic Rho/VEGF Mice

This study demonstrates the in vivo efficacy of a single dose of, an HuPTMFabVEGFi vector, as described in Section 5. 2, in juvenile transgenic Rho/VEGF mice (Tobe, 1998, IOVS 39(1):180-188), a model for the neovascular changes in the retina of humans with nAMD. Rho/VEGF mice are transgenic mice in which the rhodopsin promoter constitutively drives expression of human VEGF165 in photoreceptors, causing new vessels to sprout from the deep capillary bed of the retina and grow into the subretinal space, starting at postnatal Day 10. The production of VEGF is sustained and therefore the new vessels continue to grow and enlarge and form large nets in the subretinal space similar to those seen in humans with neovascular age-related macular degeneration. (Tobe 1998, supra).

The vector used in this study (referred to herein as “Vector 1”) is a non-replicating AAV8 vector containing a gene cassette encoding a humanized mAb antigen-binding fragment that binds and inhibits human VEGF, flanked by AAV2 inverted terminal repeats (ITRs). Expression of heavy and light chains in Vector 1 is controlled by the CB7 promoter consisting of the chicken (3-actin promoter and CMV enhancer, and the vector also comprises a chicken β-actin intron, and a rabbit β-globin polyA signal. In Vector 1, the nucleic acid sequences coding for the heavy and light chains of anti-VEGF Fab are separated by a self-cleaving furin (F)/F2A linker. Rho/VEGF mice were injected subretinally with either Vector 1 or control (n=10-17 per group) and one week later the amount of retinal neovascularization was quantitated.

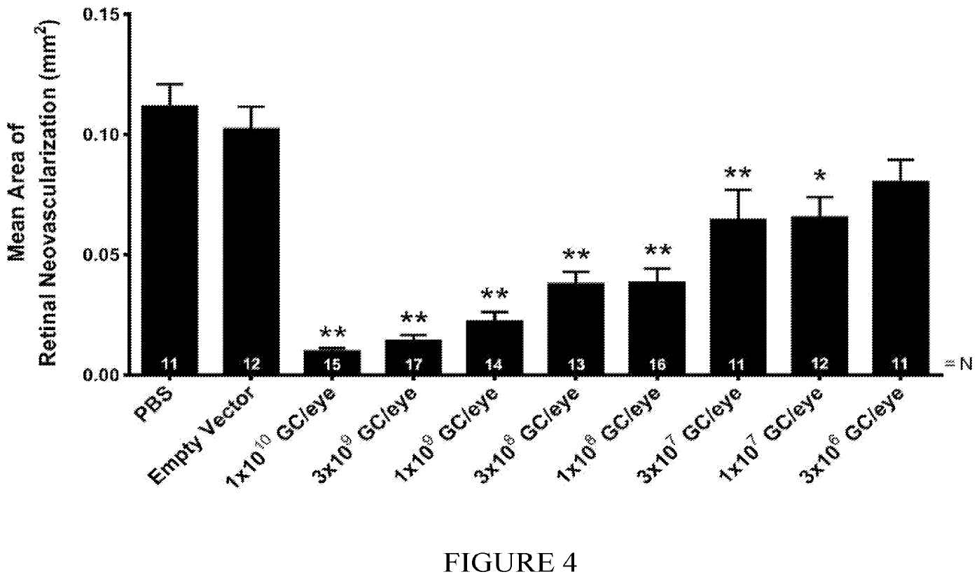

The total area of retinal neovascularization was significantly reduced (p<0.05) in Rho/VEGF mice receiving Vector 1 in a dose-dependent manner, as compared to mice receiving either phosphate buffered saline (PBS) or null AAV8 vector. The effectiveness criterion was set as a statistically significant reduction in the area of retinal neovascularization. With this criterion, a minimum dose of 1×10⁷ GC/eye of Vector 1 was determined to be efficacious for reduction of retinal neovascularization in the murine transgenic Rho/VEGF model for nAMD in human subjects (FIG. 4).

### 6.8 Example 8: A Single Dose Subretinal Administration Reduces Retinal Detachment in Double Transgenic Tet/Opsin/VEGF Mice

This study demonstrates the in vivo efficacy of a single dose of the Vector 1, to prevent retinal detachment in a transgenic mouse model of ocular neovascular disease in human subjects—Tet/opsin/VEGF mice—in which inducible expression of VEGF causes severe retinopathy and retinal detachment (Ohno-Matsui, 2002 Am. J. Pathol. 160(2):711-719). Tet/opsin/VEGF mice are transgenic mice with doxycycline inducible expression of human VEGF₁₆₅ in photoreceptors. These transgenic mice are phenotypically normal until given doxycycline in drinking water. Doxycycline induces very high photoreceptor expression of VEGF, leading to massive vascular leakage, culminating in total exudative retinal detachment in 80-90% of mice within 4 days of induction.

Tet/opsin/VEGF mice (10 per group) were injected subretinally with Vector 1 or control. Ten days after injection, doxycycline was added to the drinking water to induce VEGF expression. After 4 days, the fundus of each eye was imaged and each retina was scored as either intact, partially detached, or totally detached by an individual who had no knowledge of treatment group.

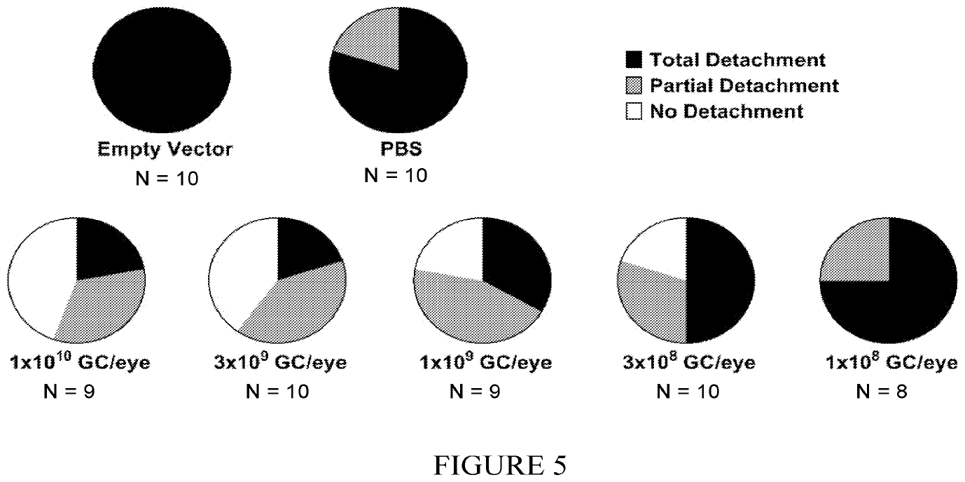

These data (shown in FIG. 5) demonstrate that treatment with Vector 1 caused a reduction in the incidence and degree of retinal detachments in Tet/opsin/VEGF mice—an animal model for ocular neovascular disease in human subjects.

### 6.9 Example 9: AAV8 Gene Therapy Expressing an Anti-VEGF Protein Strongly Suppresses Subretinal Neovascularization and Vascular Leakage in Mouse Models

In this example, the methods, results, and conclusions from the experiments described in Examples 7 and 8 are summarized.

Methods. Transgenic mice in which the rhodopsin promoter drives expression of VEGF₁₆₅ in photoreceptors (rho/VEGF mice) had a subretinal injection of Vector 1 with doses ranging from 3×10⁶-1×10¹⁰ genome copies (GC), 1×10¹⁰ GC of null vector, or PBS in one eye (n=10 per group) at post-natal day 14 (P14). At P21, the area of subretinal neovascularization (SNV) per eye was measured. Double transgenic mice with doxycycline (DOX)-inducible expression of VEGF₁₆₅ in photoreceptors (Tet/opsin/VEGF mice) had a subretinal injection of 1×10⁸-1×10¹⁰ GC of Vector 1 in one eye and no injection in the fellow eye or 1×10¹⁰ GC of null vector in one eye and PBS in the fellow eye. Ten days after injection, 2 mg/ml of DOX was added to drinking water and after 4 days fundus photos were graded for presence of total, partial, or no retinal detachment (RD). Vector 1 transgene product levels were measured one week after subretinal injection of 1×10⁸-1×10¹⁰ GC of Vector 1 in adult mice by ELISA analyses of eye homogenates.

Results. Compared to eyes of rho/VEGF mice injected null vector, those injected with ≥1×10⁷ GC of Vector 1 had significant reduction in mean area of SNV, with modest reduction in eyes injected with ≤3×10⁷ and >50% reduction in eyes injected with ≥1×10⁸ GC. Eyes injected with 3×10⁹ or 1×10¹⁰ GC had almost complete elimination of SNV. In Tet/opsin/VEGF mice, compared to the null vector group in which 100% of eyes had total RD, there was significant reduction in exudative RD in eyes injected with ≥3×10⁸ GC of Vector 1 and reduction of total detachments by 70-80% in eyes injected with 3×10⁹ or 1×10¹⁰ GC. The majority of eyes injected with ≤1×10⁹ GC of Vector 1 had protein levels below the limit of detection, but all eyes injected with 3×10⁹ or 1×10¹⁰ GC had detectable levels with mean level per eye 342.7 ng and 286.2 ng.

Conclusions. Gene therapy by subretinal injection of Vector 1 caused dose dependent suppression of SNV in rho/VEGF mice with near complete suppression with doses of 3×10⁹ or 1×10¹⁰ GC. These same doses showed robust protein product expression and markedly reduced total exudative RD in Tet/opsin/VEGF mice.

### 6.10 Example 10: Gene Therapy for Neovascular AMD: A Dose-Escalation Study to Evaluate the Safety and Tolerability of Gene Therapy with Vector 1 in Subjects with Neovascular AMD (nAMD)

Brief Summary of Study. Excessive vascular endothelial growth factor (VEGF) plays a key part in promoting neovascularization and edema in neovascular (wet) age-related macular degeneration (nAMD). VEGF inhibitors (anti-VEGF), including ranibizumab (LUCENTIS®, Genentech) and aflibercept (EYLEA®, Regeneron), have been shown to be safe and effective for treating nAMD and have demonstrated improvement in vision. However, anti-VEGF therapy is administered frequently via intravitreal injection and can be a significant burden to the patients. Vector 1 is a recombinant adeno-associated virus (AAV) gene therapy vector carrying a coding sequence for a soluble anti-VEGF protein. The long-term, stable delivery of this therapeutic protein following a one-time gene therapy treatment for nAMD could reduce the treatment burden of currently available therapies while maintaining vision with a favorable benefit:risk profile.

Detailed Description of Study. This dose-escalation study is designed to evaluate the safety and tolerability of Vector 1 gene therapy in subjects with previously treated nAMD. Three doses will be studied in approximately 18 subjects. Subjects who meet the inclusion/exclusion criteria and have an anatomic response to an initial anti VEGF injection will receive a single dose of Vector 1 administered by subretinal delivery. Vector 1 uses an AAV8 vector that contains a gene that encodes for a monoclonal antibody fragment which binds to and neutralizes VEGF activity. Safety will be the primary focus for the initial 24 weeks after Vector 1 administration (primary study period). Following completion of the primary study period, subjects will continue to be assessed until 104 weeks following treatment with Vector 1.

Dosing. Three doses will be used: 3×10⁹ GC of Vector 1, 1×10¹⁰ GC of Vector 1, and 6×10¹⁰ GC of Vector 1.

Outcome Measures. The Primary Outcome Measure will be safety—the incidence of ocular and non-ocular adverse events (AEs) and serious adverse events (SAEs)—over a time frame of 26 weeks.

Secondary Outcome Measures will include:

Safety—the incidence of ocular and non-ocular AEs and SAEs—over a time frame of 106 weeks.

Change in best corrected visual acuity (BCVA)—over a time frame of 106 weeks.

Change in central retinal thickness (CRT) as measured by SD-OCT—over a time frame of 106 weeks.

Rescue injections—the mean number of rescue injections—over a time frame of 106 weeks.

Change in choroidal neovascularization (CNV) and lesion size and leakage area CNV changes, as measured by fluorescein angiography (FA)—over a time frame of 106 weeks.

Eligibility Criteria. The following eligibility criteria apply to the study:

Minimum Age: 50 years

Maximum Age: (none)

Sex: All

Gender Based: No

Accepts Healthy Volunteers: No

Inclusion Criteria:

Patients ≥50 years with a diagnosis of subfoveal CNV secondary to AMD in the study eye receiving prior intravitreal anti-VEGF therapy.

BCVA between ≤20/100 and ≥20/400 (≤65 and ≥35 Early Treatment Diabetic Retinopathy Study [ETDRS] letters) for the first patient in each cohort followed by BCVA between ≤20/63 and ≥20/400 (≤75 and ≥35 ETDRS letters) for the rest of the cohort.

History of need for and response to anti-VEGF therapy.

Response to anti-VEGF at trial entry (assessed by SD-OCT at week 1).

Must be pseudophakic (status post cataract surgery) in the study eye.

Aspartate aminotransferase/alanine aminotransferase (AST/ALT) <2.5×upper limit of normal (ULN); total bilirubin (TB) <1.5×ULN; prothrombin time (PT) <1.5×ULN; hemoglobin (Hb) >10 g/dL (males) and >9 g/dL (females); Platelets >100×10³/μL; estimated glomerular filtration rate (eGFR) >30 mL/min/1.73 m².

Must be willing and able to provide written, signed informed consent.

Exclusion Criteria:

CNV or macular edema in the study eye secondary to any causes other than AMD.

Any condition preventing visual acuity improvement in the study eye, e.g., fibrosis, atrophy, or retinal epithelial tear in the center of the fovea.

Active or history of retinal detachment in the study eye.

Advanced glaucoma in the study eye.

History of intravitreal therapy in the study eye, such as intravitreal steroid injection or investigational product, other than anti-VEGF therapy, in the 6 months prior to screening.

Presence of an implant in the study eye at screening (excluding intraocular lens).

Myocardial infarction, cerebrovascular accident, or transient ischemic attacks within the past 6 months.

Uncontrolled hypertension (systolic blood pressure [BP]>180 mmHg, diastolic BP >100 mmHg) despite maximal medical treatment.

### 6.11 Example 11: Protocol for Treating Human Subjects

This Example relates to a gene therapy treatment for patients with neovascular (wet) age-related macular degeneration (nAMD). This Example is an updated version of Example 10. In this example, Vector 1, a replication deficient adeno-associated viral vector 8 (AAV8) carrying a coding sequence for a soluble anti-VEGF Fab protein (as described in Example 7), is administered to patients with nAMD. The goal of the gene therapy treatment is to slow or arrest the progression of retinal degeneration and to slow or prevent loss of vision with minimal intervention/invasive procedures.

Dosing & Route of Administration. A volume of 250 μL of Vector 1 is administered as a single dose via subretinal delivery in the eye of a subject in need of treatment. The subject receives a dose of 3×10⁹ GC/eye, 1×10¹⁰ GC/eye, or 6×10¹⁰ GC/eye.

Subretinal delivery is performed by a retinal surgeon with the subject under local anesthesia. The procedure involves a standard 3-port pars plana vitrectomy with a core vitrectomy followed by subretinal delivery of Vector 1 into the subretinal space by a subretinal cannula (38 gauge). The delivery is automated via the vitrectomy machine to deliver 250 μL to the subretinal space.

Gene therapy can be administered in combination with one or more therapies for the treatment of wetAMD. For example, gene therapy is administered in combination with laser coagulation, photodynamic therapy with verteporfin, and intravitreal with anti-VEGF agent, including but not limited to pegaptanib, ranibizumab, aflibercept, or bevacizumab.

Starting at about 4 weeks post-Vector 1 administration, a patient may receive intravitreal ranibizumab rescue therapy in the affected eye.

Patient Subpopulations. Suitable patients may include those:

Having a diagnosis of nAMD;

Responsive to anti-VEGF therapy;

Requiring frequent injections of anti-VEGF therapy;

Males or females aged 50 years or above;

Having a BCVA ≤20/100 and ≥20/400 (≤65 and ≥35 ETDRS letters) in the affected eye;

Having a BCVA between ≤20/63 and ≥20/400 (≤75 and ≥35 ETDRS letters);

Having a documented diagnosis of subfoveal CNV secondary to AMD in the affected eye;

Having CNV lesion characteristics as follows: lesion size less than 10 disc areas (typical disc area is 2.54 mm²), blood and/or scar <50% of the lesion size;

Having received at least 4 intravitreal injections of an anti-VEGF agent for treatment of nAMD in the affected eye in the 8 months (or less) prior to treatment, with anatomical response documented on SD-OCT; and/or

Having subretinal or intraretinal fluid present in the affected eye, evidenced on SD-OCT.

Prior to treatment, patients are screened and one or more of the following criteria may indicate this therapy is not suitable for the patient:

CNV or macular edema in the affected eye secondary to any causes other than AMD;

Blood occupying ≥50% of the AMD lesion or blood >1.0 mm2 underlying the fovea in the affected eye;

Any condition preventing VA improvement in the affected eye, e.g., fibrosis, atrophy, or retinal epithelial tear in the center of the fovea;

Active or history of retinal detachment in the affected eye;

Advanced glaucoma in the affected eye;

Any condition in the affected eye that may increase the risk to the subject, require either medical or surgical intervention to prevent or treat vision loss, or interfere with study procedures or assessments;

History of intraocular surgery in the affected eye within 12 weeks prior to screening (Yttrium aluminum garnet capsulotomy may be permitted if performed >10 weeks prior to the screening visit.);

History of intravitreal therapy in the affected eye, such as intravitreal steroid injection or investigational product, other than anti-VEGF therapy, in the 6 months prior to screening;

Presence of an implant in the affected eye at screening (excluding intraocular lens).

History of malignancy requiring chemotherapy and/or radiation in the 5 years prior to screening (Localized basal cell carcinoma may be permitted.);

History of therapy known to have caused retinal toxicity, or concomitant therapy with any drug that may affect visual acuity or with known retinal toxicity, e.g., chloroquine or hydroxychloroquine;

Ocular or periocular infection in the affected eye that may interfere with the surgical procedure;

Myocardial infarction, cerebrovascular accident, or transient ischemic attacks within the past 6 months of treatment;

Uncontrolled hypertension (systolic blood pressure [BP]>180 mmHg, diastolic BP >100 mmHg) despite maximal medical treatment;

Any concomitant treatment that may interfere with ocular surgical procedure or healing process;

Known hypersensitivity to ranibizumab or any of its components or past hypersensitivity to agents like Vector 1;

Any serious or unstable medical or psychological condition that, in the opinion of the Investigator, would compromise the subject's safety or successful participation in the study.

Aspartate aminotransferase (AST)/alanine aminotransferase (ALT) >2.5×upper limit of normal (ULN)

Total bilirubin >1.5×ULN unless the subject has a previously known history of Gilbert's syndrome and a fractionated bilirubin that shows conjugated bilirubin <35% of total bilirubin

Prothrombin time (PT) >1.5×ULN

Hemoglobin <10 g/dL for male subjects and <9 g/dL for female subjects

Platelets <100×10³/μL

Estimated glomerular filtration rate (GFR) <30 mL/min/1.73 m²

Starting at about 4 weeks post-gene therapy administration, a patient may receive intravitreal ranibizumab rescue therapy in the affected eye for disease activity if 1 or more of the following rescue criteria apply:

Vision loss of ≥5 letters (per Best Corrected Visual Acuity [BCVA]) associated with accumulation of retinal fluid on Spectral Domain Optical Coherence Tomography (SD-OCT)

Choroidal neovascularization (CNV)-related increased, new, or persistent subretinal or intraretinal fluid on SD-OCT

New ocular hemorrhage

Further rescue injections may be deferred per the treating physician's discretion if one of the following sets of findings occur:

Visual acuity is 20/20 or better and central retinal thickness is “normal” as assessed by SD-OCT, or

Visual acuity and SD-OCT are stable after 2 consecutive injections.

If injections are deferred, they will be resumed if visual acuity or SD-OCT get worse per the criteria above.

Measuring Clinical Objectives. Primary clinical objectives include slowing or arresting the progression of retinal degeneration and slowing or preventing loss of vision. Clinical objectives are indicated by the elimination of or reduction in the number of rescue treatments using standard of care, for example, intravitreal injections with anti-VEGF agents, including but not limited to pegaptanib, ranibizumab, aflibercept, or bevacizumab. Clinical objectives are also indicated by a decrease or prevention of vision loss and/or a decrease or prevention of retinal detachment.

Clinical objectives are determined by measuring BCVA (Best-Corrected Visual Acuity), intraocular pressure, slit lamp biomicroscopy, indirect ophthalmoscopy, and/or SD-OCT (SD-Optical Coherence Tomography). In particular, clinical objectives are determined by measuring mean change from baseline in BCVA over time, measuring the gain or loss of ≥15 letters compared to baseline as per BCVA, measuring mean change from baseline in CRT as measured by SD-OCT over time, measuring mean number of ranibizumab rescue injections over time, measuring time to 1^{st} rescue ranibizumab injection, measuring mean change from baseline in CNV and lesion size and leakage area based on FA over time, measuring mean change from baseline in aqueous aVEGF protein over time, performing vector shedding analysis in serum and urine, and/or measuring immunogenicity to Vector 1, i.e., measuring Nabs to AAV, measuring binding antibodies to AAV, measuring antibodies to aVEGF, and/or performing ELISpot.

Clinical objectives are also determined by measuring the mean change from baseline over time in area of geographic atrophy per fundus autofluorescence (FAF), measuring the incidence of new area of geographic atrophy by FAF (in subjects with no geographic atrophy at baseline, measuring the proportion of subjects gaining or losing ≥5 and ≥10 letters, respectively, compared with baseline as per BCVA, measuring the proportion of subjects who have a reduction of 50% in rescue injections compared with previous year, measuring the proportion of subjects with no fluid on SD-OCT.

Improvement/efficacy resulting from Vector 1 administration can be assessed as a defined mean change in baseline in visual acuity at about 4 weeks, 12 weeks, 6 months, 12 months, 24 months, 36 months, or at other desired timepoints. Treatment with Vector 1 can result in a 5%, 10%, 15%, 20%, 30%, 40%, 50% or more increase in visual acuity from baseline. Improvements/efficacy can be assessed as mean change from baseline in central retinal thickness (CRT) as measured by spectral domain optical coherence tomography (SD-OCT) at 4 weeks, 12 weeks, 6 months, 12 months, 24 months and 36 months. Treatment with Vector 1 can result in a 5%, 10%, 15%, 20%, 30%, 40%, 50% or more increase central retinal thickness from baseline.

### EQUIVALENTS

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

1-7. (canceled)

8. An anti-human vascular endothelial growth factor (hVEGF) antigen-binding fragment produced by human retinal cells.

9. The anti-hVEGF antigen-binding fragment of claim 8, which is produced by human photoreceptor cells.

10. The anti-hVEGF antigen-binding fragment of claim 8, which is glycosylated.

11. The anti-hVEGF antigen-binding fragment of claim 10, which contains a α2,6-sialylated glycan.

12. The anti-hVEGF antigen-binding fragment of claim 10, which does not contain NeuGc.

13. The anti-hVEGF antigen-binding fragment of claim 8, which contains a tyrosine-sulfation.

14. The anti-hVEGF antigen-binding fragment of claim 8, which is a Fab, F(ab′)₂, or single chain variable domain (scFv).

15. The anti-hVEGF antigen-binding fragment of claim 8, which comprises a heavy chain comprising the amino acid sequence of SEQ ID NO. 1 or SEQ ID NO. 3, and a light chain comprising the amino acid sequence of SEQ ID NO. 2, or SEQ ID NO. 4.

16. The anti-hVEGF antigen-binding fragment of claim 8, which comprises light chain CDRs 1-3 of SEQ ID NOs: 14-16 and heavy chain CDRs 1-3 of SEQ ID NOs: 17-19 or SEQ ID NOs: 20, 18, and 21.

17. A glycosylated anti-hVEGF antigen-binding fragment, which:
(a) contains a α2,6-sialylated glycan;
(b) does not contain NeuGc; and/or
(c) contains a tyrosine-sulfation.

18. The glycosylated anti-hVEGF antigen-binding fragment of claim 17, which is a Fab, F(ab′)₂, or single chain variable domain (scFv).

19. The glycosylated anti-hVEGF antigen-binding fragment of claim 17, which comprises a heavy chain comprising the amino acid sequence of SEQ ID NO. 1 or SEQ ID NO. 3, and a light chain comprising the amino acid sequence of SEQ ID NO. 2, or SEQ ID NO. 4.

20. The glycosylated anti-hVEGF antigen-binding fragment of claim 17, which comprises light chain CDRs 1-3 of SEQ ID NOs: 14-16 and heavy chain CDRs 1-3 of SEQ ID NOs:17-19 or SEQ ID NOs: 20, 18, and 21.

21. An anti-hVEGF antigen-binding fragment, which contains a tyrosine-sulfation.

22. A method of treating a human subject diagnosed with neovascular age-related macular degeneration (nAMD), comprising delivering to the retina of said human subject a therapeutically effective amount of the anti-hVEGF antigen-binding fragment of claim 8.

23. The method of claim 22, wherein the anti-hVEGF antigen-binding fragment is expressed from an AAV8-based viral vector.

24. A method of treating a human subject diagnosed with nAMD, comprising delivering to the retina of said human subject a therapeutically effective amount of the glycosylated anti-hVEGF antigen-binding fragment of claim 17.

25. The method of claim 24, wherein the glycosylated anti-hVEGF antigen-binding fragment is expressed from an AAV8-based viral vector.

26. A method of treating a human subject diagnosed with neovascular age-related macular degeneration (nAMD), comprising delivering to the retina of said human subject a therapeutically effective amount of the anti-hVEGF antigen-binding fragment of claim 21.

27. The method of claim 26, wherein the anti-hVEGF antigen-binding fragment is expressed from an AAV8-based viral vector.

28. A pharmaceutical composition suitable for subretinal administration comprising a suspension of a recombinant viral vector encoding an anti-hVEGF antigen-binding fragment in a formulation buffer comprising a physiologically compatible aqueous buffer and a surfactant wherein the recombinant viral vector in the composition is capable of transducing human retinal cells upon subretinal administration.

29. A pharmaceutical composition suitable for subretinal administration comprising a suspension of a recombinant viral vector encoding an anti-hVEGF antigen-binding fragment in a formulation buffer comprising a physiologically compatible aqueous buffer and a surfactant wherein the recombinant viral vector in the composition is capable of transducing human retinal cells upon subretinal administration.

30. A method of manufacturing a recombinant viral vector comprising providing a host cell stably transformed with sequences encoding an anti-hVEGF antigen-binding fragment, wherein the recombinant viral vector is capable of transducing human retinal cells upon subretinal administration.

31. A method of manufacturing a recombinant viral vector comprising providing a host cell stably transformed with sequences encoding an anti-hVEGF antigen-binding fragment, wherein the recombinant viral vector is capable of transducing human retinal cells upon subretinal administration.


SEQUENCE LISTING (37 sequences)

SEQ ID NO: 1 - protein, 214 aa, Artificial Sequence
  Ranibizumab Fab Amino Acid Sequence - light chain
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 231 aa, Artificial Sequence
  Ranibizumab Fab Amino Acid Sequence - heavy chain
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 3 - protein, 214 aa, Artificial Sequence
  Bevacizumab Fab Amino Acid Sequence - Light chain
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 231 aa, Artificial Sequence
  Bevacizumab Fab Amino Acid Sequence - Heavy chain
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 5 - protein, 26 aa, Artificial Sequence
  VEGF-A signal peptide
MNFLLSWVHWSLALLLYLHHAKWSQA

SEQ ID NO: 6 - protein, 29 aa, Artificial Sequence
  Fibulin-1 signal peptide
MERAAPSRRVPLPLLLLGGLALLAAGVDA

SEQ ID NO: 7 - protein, 19 aa, Artificial Sequence
  Vitronectin signal peptide
MAPLRPLLILALLAWVALA

SEQ ID NO: 8 - protein, 18 aa, Artificial Sequence
  Complement Factor H signal peptide
MRLLAKIICLMLWAICVA

SEQ ID NO: 9 - protein, 19 aa, Artificial Sequence
  Opticin signal peptide
MRLLAFLSLLALVLQETGT

SEQ ID NO: 10 - DNA, 728 bp, Artificial Sequence
  Bevacizumab cDNA - Light chain
gctagcgccaccatgggctggtcctgcatcatcctgttcctggtggccaccgccaccggcgtgcactccgacatccagatgacccagtccccctcctccctgtccgcctccgtgggcgaccgggtgaccatcacctgctccgcctcccaggacatctccaactacctgaactggtaccagcagaagcccggcaaggcccccaaggtgctgatctacttcacctcctccctgcactccggcgtgccctcccggttctccggctccggctccggcaccgacttcaccctgaccatctcctccctgcagcccgaggacttcgccacctactactgccagcagtactccaccgtgccctggaccttcggccagggcaccaaggtggagatcaagcggaccgtggccgccccctccgtgttcatcttccccccctccgacgagcagctgaagtccggcaccgcctccgtggtgtgcctgctgaacaacttctacccccgggaggccaaggtgcagtggaaggtggacaacgccctgcagtccggcaactcccaggagtccgtgaccgagcaggactccaaggactccacctactccctgtcctccaccctgaccctgtccaaggccgactacgagaagcacaaggtgtacgcctgcgaggtgacccaccagggcctgtcctcccccgtgaccaagtccttcaaccggggcgagtgctgagcggccgcctcgag

SEQ ID NO: 11 - DNA, 1440 bp, Artificial Sequence
  Bevacizumab cDNA - heavy chain
gctagcgccaccatgggctggtcctgcatcatcctgttcctggtggccaccgccaccggcgtgcactccgaggtgcagctggtggagtccggcggcggcctggtgcagcccggcggctccctgcggctgtcctgcgccgcctccggctacaccttcaccaactacggcatgaactgggtgcggcaggcccccggcaagggcctggagtgggtgggctggatcaacacctacaccggcgagcccacctacgccgccgacttcaagcggcggttcaccttctccctggacacctccaagtccaccgcctacctgcagatgaactccctgcgggccgaggacaccgccgtgtactactgcgccaagtacccccactactacggctcctcccactggtacttcgacgtgtggggccagggcaccctggtgaccgtgtcctccgcctccaccaagggcccctccgtgttccccctggccccctcctccaagtccacctccggcggcaccgccgccctgggctgcctggtgaaggactacttccccgagcccgtgaccgtgtcctggaactccggcgccctgacctccggcgtgcacaccttccccgccgtgctgcagtcctccggcctgtactccctgtcctccgtggtgaccgtgccctcctcctccctgggcacccagacctacatctgcaacgtgaaccacaagccctccaacaccaaggtggacaagaaggtggagcccaagtcctgcgacaagacccacacctgccccccctgccccgcccccgagctgctgggcggcccctccgtgttcctgttcccccccaagcccaaggacaccctgatgatctcccggacccccgaggtgacctgcgtggtggtggacgtgtcccacgaggaccccgaggtgaagttcaactggtacgtggacggcgtggaggtgcacaacgccaagaccaagccccgggaggagcagtacaactccacctaccgggtggtgtccgtgctgaccgtgctgcaccaggactggctgaacggcaaggagtacaagtgcaaggtgtccaacaaggccctgcccgcccccatcgagaagaccatctccaaggccaagggccagccccgggagccccaggtgtacaccctgcccccctcccgggaggagatgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtcccggtggcagcagggcaacgtgttctcctgctccgtgatgcacgaggccctgcacaaccactacacccagaagtccctgtccctgtcccccggcaagtgagcggccgcc

SEQ ID NO: 12 - DNA, 733 bp, Artificial Sequence
  Ranibizumab cDNA (Light chain comprising a signal sequence)
gagctccatggagtttttcaaaaagacggcacttgccgcactggttatgggttttagtggtgcagcattggccgatatccagctgacccagagcccgagcagcctgagcgcaagcgttggtgatcgtgttaccattacctgtagcgcaagccaggatattagcaattatctgaattggtatcagcagaaaccgggtaaagcaccgaaagttctgatttattttaccagcagcctgcatagcggtgttccgagccgttttagcggtagcggtagtggcaccgattttaccctgaccattagcagcctgcagccggaagattttgcaacctattattgtcagcagtatagcaccgttccgtggacctttggtcagggcaccaaagttgaaattaaacgtaccgttgcagcaccgagcgtttttatttttccgcctagtgatgaacagctgaaaagcggcaccgcaagcgttgtttgtctgctgaataatttttatccgcgtgaagcaaaagtgcagtggaaagttgataatgcactgcagagcggtaatagccaagaaagcgttaccgaacaggatagcaaagatagcacctatagcctgagcagcaccctgaccctgagcaaagcagattatgaaaaacacaaagtgtatgcctgcgaagttacccatcagggtctgagcagtccggttaccaaaagttttaatcgtggcgaatgctaatagaagcttggtacc

SEQ ID NO: 13 - DNA, 779 bp, Artificial Sequence
  Ranibizumab cDNA (Heavy chain comprising a signal sequence)
gagctcatatgaaatacctgctgccgaccgctgctgctggtctgctgctcctcgctgcccagccggcgatggccgaagttcagctggttgaaagcggtggtggtctggttcagcctggtggtagcctgcgtctgagctgtgcagcaagcggttatgattttacccattatggtatgaattgggttcgtcaggcaccgggtaaaggtctggaatgggttggttggattaatacctataccggtgaaccgacctatgcagcagattttaaacgtcgttttacctttagcctggataccagcaaaagcaccgcatatctgcagatgaatagcctgcgtgcagaagataccgcagtttattattgtgccaaatatccgtattactatggcaccagccactggtatttcgatgtttggggtcagggcaccctggttaccgttagcagcgcaagcaccaaaggtccgagcgtttttccgctggcaccgagcagcaaaagtaccagcggtggcacagcagcactgggttgtctggttaaagattattttccggaaccggttaccgtgagctggaatagcggtgcactgaccagcggtgttcatacctttccggcagttctgcagagcagcggtctgtatagcctgagcagcgttgttaccgttccgagcagcagcctgggcacccagacctatatttgtaatgttaatcataaaccgagcaataccaaagtggataaaaaagttgagccgaaaagctgcgataaaacccatctgtaatagggtacc

SEQ ID NO: 14 - protein, 11 aa, Artificial Sequence
  Bevacizumab and Ranibizumab Light Chain CDR1
SASQDISNYLN

SEQ ID NO: 15 - protein, 7 aa, Artificial Sequence
  Bevacizumab and Ranibizumab Light Chain CDR2
FTSSLHS

SEQ ID NO: 16 - protein, 9 aa, Artificial Sequence
  Bevacizumab and Ranibizumab Light Chain CDR3
QQYSTVPWT

SEQ ID NO: 17 - protein, 10 aa, Artificial Sequence
  Bevacizumab Heavy Chain CDR1
GYTFTNYGMN

SEQ ID NO: 18 - protein, 17 aa, Artificial Sequence
  Bevacizumab Heavy Chain CDR2
WINTYTGEPTYAADFKR

SEQ ID NO: 19 - protein, 14 aa, Artificial Sequence
  Bevacizumab Heavy Chain CDR3
YPHYYGSSHWYFDV

SEQ ID NO: 20 - protein, 10 aa, Artificial Sequence
  Ranibizumab Heavy Chain CDR1
GYDFTHYGMN

SEQ ID NO: 21 - protein, 14 aa, Artificial Sequence
  Ranibizumab Heavy Chain CDR3
YPYYYGTSHWYFDV

SEQ ID NO: 22 - protein, 18 aa, Artificial Sequence
  Albumin signal peptide
MKWVTFISLLFLFSSAYS

SEQ ID NO: 23 - protein, 18 aa, Artificial Sequence
  Chymotrypsinogen signal peptide
MAFLWLLSCWALLGTTFG

SEQ ID NO: 24 - protein, 20 aa, Artificial Sequence
  Interleukin-2 signal peptide
MYRMQLLSCIALILALVTNS

SEQ ID NO: 25 - protein, 15 aa, Artificial Sequence
  Trypsinogen-2 signal peptide
MNLLLILTFVAAAVA

SEQ ID NO: 26 - protein, 19 aa, Artificial Sequence
  F2A site
LLNFDLLKLAGDVESNPGP

SEQ ID NO: 27 - protein, 21 aa, Artificial Sequence
  Linker T2A
GSGEGRGSLLTCGDVEENPGP

SEQ ID NO: 28 - protein, 22 aa, Artificial Sequence
  Linker P2A
GSGATNFSLLKQAGDVEENPGP

SEQ ID NO: 29 - protein, 23 aa, Artificial Sequence
  Linker E2A
GSGQCTNYALLKLAGDVESNPGP

SEQ ID NO: 30 - protein, 25 aa, Artificial Sequence
  Linker F2A
GSGVKQTLNFDLLKLAGDVESNPGP

SEQ ID NO: 31 - protein, 4 aa, Artificial Sequence
  Furin linker
RKRR

SEQ ID NO: 32 - protein, 4 aa, Artificial Sequence
  Furin linker
RRRR

SEQ ID NO: 33 - protein, 4 aa, Artificial Sequence
  Furin linker
RRKR

SEQ ID NO: 34 - protein, 4 aa, Artificial Sequence
  Furin linker
RKKR

SEQ ID NO: 35 - protein, 4 aa, Artificial Sequence
  Furin linker
  MISC_FEATURE (2)..(2): X = any amino acid
  MISC_FEATURE (3)..(3): X = Lys or Arg
RXXR

SEQ ID NO: 36 - protein, 4 aa, Artificial Sequence
  Furin linker
  MISC_FEATURE (2)..(2): X = any amino acid
RXKR

SEQ ID NO: 37 - protein, 4 aa, Artificial Sequence
  Furin linker
  MISC_FEATURE (2)..(2): X = any amino acid
RXRR